SEQUENCE LISTING (130 sequences)

SEQ ID NO: 1 - DNA, 1521 bp, synthetic construct
  misc_feature 1..1521: note = canine with a His Tag
  source 1..1521: mol_type = other DNA
gtgctgcccgccaagcccgagaacatcagctgcatcttctactacgaggagaacttcacctgcacctggagccccgagaaggaggccagctacacctggtacaaggtgaagagaacctacagctacggctacaagagcgacatctgcagcaccgacaacagcaccagaggcaaccacgccagctgcagcttcctgccccccaccatcaccaaccccgacaactacaccatccaggtggaggcccagaacgccgacggcatcatgaagagcgacatcacctactggaacctggacgccatcatgaagatcgagccccccgagatcttcagcgtgaagagcgtgctgggcatcaagagaatgctgcagatcaagtggatcagacccgtgctggccccccacagcagcaccctgaagtacaccctgagattcagaaccatcaacagcgcctactggatggaggtgaacttcaccaaggaggacatcgacagagacgagacctacaacctgaccgagctgcaggccttcaccgagtacgtgatgaccctgagatgcgcccccgccgagagcatgttctggagcggctggagccaggagaaggtgggcaccaccgaggaggaggccccctacggcctggacctgtggagagtgctgaagcccgccatggtggacggcagaagacccgtgcagctgatgtggaagaaggccaccggcgcccccgtgctggagaaggccctgggctacaacatctggtacttccccgagaacaacaccaacctgaccgagaccgtgaacaccaccaaccagacccacgagctgtacctgggcggcaagacctactgggtgtacgtggtgagctacaacagcctgggcgagagccccgtggccaccctgagaatccccgccctgaacgagaagaccttccagtgcatcgaggccatgcaggcctgcctgacccaggaccagctggtggtggagtggcagagcagcgcccccgaggtggacacctggatggtggagtggttccccgacgtggacagcgagcccagcagcttcagctgggagagcgtgagccaggccagaaactggaccatccagaaggacgagctgaagcccctgtggtgctacaacatcagcgtgtaccccgtgctgagagacagagtgggccagccctacagcacccaggcctacgtgcaggagggcatccccagcgccggccccgtgacccaggccgacagcatcggcgtgaagaccgtgaccatcacctggaaggagatccccaagagcaagagaaacggcttcatcaagaactacaccatcttctaccaggccgaggacggcaaggagttcagcaagaccgtgaacagcaacatcctgcagtacagactggagagcctgaccagaagaaccagctacagcctgcaggtgatggccagcaccaacgccggcggcaccaacggcaccaagatcaacttcaagaccctgagcatcagccaccaccaccaccaccaccaccaccaccac

SEQ ID NO: 2 - protein, 507 aa, synthetic construct
  REGION 1..507: note = canine with a His Tag
VLPAKPENISCIFYYEENFTCTWSPEKEASYTWYKVKRTYSYGYKSDICSTDNSTRGNHASCSFLPPTITNPDNYTIQVEAQNADGIMKSDITYWNLDAIMKIEPPEIFSVKSVLGIKRMLQIKWIRPVLAPHSSTLKYTLRFRTINSAYWMEVNFTKEDIDRDETYNLTELQAFTEYVMTLRCAPAESMFWSGWSQEKVGTTEEEAPYGLDLWRVLKPAMVDGRRPVQLMWKKATGAPVLEKALGYNIWYFPENNTNLTETVNTTNQTHELYLGGKTYWVYVVSYNSLGESPVATLRIPALNEKTFQCIEAMQACLTQDQLVVEWQSSAPEVDTWMVEWFPDVDSEPSSFSWESVSQARNWTIQKDELKPLWCYNISVYPVLRDRVGQPYSTQAYVQEGIPSAGPVTQADSIGVKTVTITWKEIPKSKRNGFIKNYTIFYQAEDGKEFSKTVNSNILQYRLESLTRRTSYSLQVMASTNAGGTNGTKINFKTLSISHHHHHHHHHH

SEQ ID NO: 3 - protein, 5 aa, Mus musculus
DYNMD

SEQ ID NO: 4 - protein, 5 aa, Mus musculus
DYSIN

SEQ ID NO: 5 - protein, 5 aa, Mus musculus
NTFIH

SEQ ID NO: 6 - protein, 5 aa, Mus musculus
NTYMH

SEQ ID NO: 7 - protein, 5 aa, Mus musculus
DYGMN

SEQ ID NO: 8 - protein, 7 aa, Mus musculus
TFGRGVG

SEQ ID NO: 9 - protein, 5 aa, Mus musculus
SYGMS

SEQ ID NO: 10 - protein, 17 aa, Mus musculus
HINPNNGGILYNQKFKG

SEQ ID NO: 11 - protein, 17 aa, Mus musculus
HINPNNGGTLYNQKFKD

SEQ ID NO: 12 - protein, 17 aa, Mus musculus
HINPNIGGTLYNQKFKG

SEQ ID NO: 13 - protein, 17 aa, Mus musculus
HINPNTGGTIYNQKFKG

SEQ ID NO: 14 - protein, 17 aa, Mus musculus
VINPNYGTSSHNQKFKG

SEQ ID NO: 15 - protein, 17 aa, Mus musculus
RIDPANGNTKYAPKFQG

SEQ ID NO: 16 - protein, 17 aa, Mus musculus
RIDPANGNSKYAPKFQG

SEQ ID NO: 17 - protein, 17 aa, Mus musculus
IINPNYGTASSNPKFKD

SEQ ID NO: 18 - protein, 16 aa, Mus musculus
HIWWDDDKYYNPALKS

SEQ ID NO: 19 - protein, 17 aa, Mus musculus
TISNGGSYTYYPDSVKG

SEQ ID NO: 20 - protein, 11 aa, Mus musculus
WAPLVRQPYWY

SEQ ID NO: 21 - protein, 14 aa, Mus musculus
WAPLLRQPYWYFDV

SEQ ID NO: 22 - protein, 14 aa, Mus musculus
WAQLQRQPYWYFDV

SEQ ID NO: 23 - protein, 14 aa, Mus musculus
WAQLLRQPYWYFDV

SEQ ID NO: 24 - protein, 10 aa, Mus musculus
ALDDYYFYWY

SEQ ID NO: 25 - protein, 7 aa, Mus musculus
YYYVSSY

SEQ ID NO: 26 - protein, 10 aa, Mus musculus
YYYVSSHFDV

SEQ ID NO: 27 - protein, 13 aa, Mus musculus
AFDGYYFYWYFDV

SEQ ID NO: 28 - protein, 14 aa, Mus musculus
IAGGLRRAPYAMDS

SEQ ID NO: 29 - protein, 6 aa, Mus musculus
NEPPDY

SEQ ID NO: 30 - protein, 11 aa, Mus musculus
KASQDINSYLN

SEQ ID NO: 31 - protein, 11 aa, Mus musculus
KASQDINSYFN

SEQ ID NO: 32 - protein, 11 aa, Mus musculus
KASQSVTNDVT

SEQ ID NO: 33 - protein, 16 aa, Mus musculus
RSSQSLVHTNGNTYLH

SEQ ID NO: 34 - protein, 16 aa, Mus musculus
RSSQSLIHTNGNTYLH

SEQ ID NO: 35 - protein, 11 aa, Mus musculus
RASENIYSSLA

SEQ ID NO: 36 - protein, 12 aa, Mus musculus
TASSSVGSSYLH

SEQ ID NO: 37 - protein, 7 aa, Mus musculus
KVSNRFS

SEQ ID NO: 38 - protein, 7 aa, Mus musculus
RVNRLVD

SEQ ID NO: 39 - protein, 7 aa, Mus musculus
RANRLVD

SEQ ID NO: 40 - protein, 7 aa, Mus musculus
RADRLVD

SEQ ID NO: 41 - protein, 7 aa, Mus musculus
SASNRYT

SEQ ID NO: 42 - protein, 7 aa, Mus musculus
YASNRYT

SEQ ID NO: 43 - protein, 7 aa, Mus musculus
AATNLAD

SEQ ID NO: 44 - protein, 7 aa, Mus musculus
DTSNLAS

SEQ ID NO: 45 - protein, 9 aa, Mus musculus
LQYDEFPLT

SEQ ID NO: 46 - protein, 9 aa, Mus musculus
SQTTHVPLT

SEQ ID NO: 47 - protein, 9 aa, Mus musculus
QQDYSSPWT

SEQ ID NO: 48 - protein, 9 aa, Mus musculus
QQDYSSPFT

SEQ ID NO: 49 - protein, 9 aa, Mus musculus
HQYHRSPYT

SEQ ID NO: 50 - protein, 9 aa, Mus musculus
SQSTHVPLT

SEQ ID NO: 51 - protein, 9 aa, Mus musculus
QHFRDTPPT

SEQ ID NO: 52 - protein, 123 aa, Mus musculus
EVQLQQSGPELVKPGASVKIPCKASGYTFTDYNMDWVKQSHGKSLEWIGHINPNNGGILYNQKFKGKATLTVDRSSNTAYMELRSLTSEDTAVYFCARWAPLVRQPYWYFDVWGTGTTVTVSS

SEQ ID NO: 53 - protein, 107 aa, Mus musculus
DIKMTQSPSSIFASLGERVTITCKASQDINSYLNWFQQKPGKSPKTLIYRADRLVDGVPSRFSGSGSGQDYSLTISSLEYEDMGIYYCLQYDEFPLTFGAGTKLELN

SEQ ID NO: 54 - protein, 123 aa, Mus musculus
EVQLQQSGPELVKPGSSVKIPCKASGYTFTDYNMDWVKQSHGKSLEWIGHINPNIGGTLYNQKFKGKATLSVDKSSSTAYMELRSLTSEDTAVYYCARWAQLQRQPYWYFDVWGPGTTVTVSS

SEQ ID NO: 55 - protein, 107 aa, Mus musculus
DIKMTQSPSSMYASLGERVTITCKASQDINSYLNWFQQKPGKSPKTLIYRVNRLVDGVPSRFSGSGSGQDYSLTISSLEYEDMGIYYCLQYDEFPLTFGAGTKLELK

SEQ ID NO: 56 - protein, 123 aa, Mus musculus
EVQLQQSGPELVKPGASVKIPCKASGYTFTDYNMDWVKQSHGKSLEWIGHINPNTGGTIYNQKFKGKATLTVDKSSSTAYMDLRSLTSEDTAVYYCARWAQLLRQPYWYFDVWGTGTTVTVSS

SEQ ID NO: 57 - protein, 107 aa, Mus musculus
DIKMTQSPSSMYASLGERVTITCKASQDINSYFNWVQQKPGKSPKTLIFRANRLVDGVPSRFSGSGSGQDYSLTINSLEYEDMGIYYCLQYDEFPLTFGAGTKLELK

SEQ ID NO: 58 - protein, 123 aa, Mus musculus
EVQLQQSGPELVKPGASMKIPCKTSGYTFTDYNMDWVKQSHGKSLEWIGHINPNNGGTLYNQKFKDKATLTVDRSSNTAYMELRSLTSEDTAVYYCARWAPLLRQPYWYFDVWGTGTTVTVSS

SEQ ID NO: 59 - protein, 107 aa, Mus musculus
DIKMTQSPSSMYASLGERVTITCKASQDINSYLNWFQQKPGKSPKTLIYRADRLVDGVPSRFSGSGSGQDYSLTISSLEYEDMGIYYCLQYDEFPLTFGDGTKLELN

SEQ ID NO: 60 - protein, 119 aa, Mus musculus
EVQLQQSVAELVRPGASVKLSCTASGFNIKNTYMHWVKERPEQGLEWIGRIDPANGNSKYAPKFQGKATITTDTSSNTAYLQLSSLTSEDTAIYYCARYYYVSSHFDVWGTGTTVTVSS

SEQ ID NO: 61 - protein, 107 aa, Mus musculus
SIVMTQTPKFLLVSAGDRVTITCKASQSVTNDVTWYQQKPGQSPKLLIYYASNRYTGVPDRFTGSGYGTDFTFTISTVQAEDLAVYFCQQDYSSPFTFGSGTKLEIK

SEQ ID NO: 62 - protein, 119 aa, Mus musculus
EVQLQQSVAELVRPGASVKLSCTASGFNIKNTFIHWVKQRPEQGLEWIGRIDPANGNTKYAPKFQGRATLTADTSSNTAYLQLSSLTSEDTAIYYCARYYYVSSYFDVWGTGTTVTVSS

SEQ ID NO: 63 - protein, 107 aa, Mus musculus
SIVMTQTPKFLLVSAGDRVTITCKASQSVTNDVTWYQQKPGQSPKLLIFSASNRYTGVPDRFTGSGYGTDFTFTISTVQAEDLAVYFCQQDYSSPWTFGGGTKLEIK

SEQ ID NO: 64 - protein, 122 aa, Mus musculus
EFQLQQSGPELVKPGASVKISCKASGYSFTDYGMNWLKQINGKSLEWIAIINPNYGTASSNPKFKDKATLTVDHSSSTAYMQLSSLTSEDSAVYYCARAFDGYYFYWYFDVWGTGTTVTVSS

SEQ ID NO: 65 - protein, 112 aa, Mus musculus
DVVMTQTPLSLPVSLGDQASISCRSSQSLIHTNGNTYLHWYLQKPGQSPKLLIYKVSNRFSGVPDRFSGSGSGTDFTLKISRLEAEDLGVYFCSQSTHVPLTFGAGTKLELR

SEQ ID NO: 66 - protein, 122 aa, Mus musculus
EFQLQQSGPELVKPGASVKISCKASGYSFTDYSINWVKQSNGKSLEWIGVINPNYGTSSHNQKFKGKATMTVDQSSSTAYMQLNSLTSEDSAVYYCARALDDYYFYWYFDVWGIGTTVTVSS

SEQ ID NO: 67 - protein, 112 aa, Mus musculus
DVVMTQTPLSLPVSLGDQASISCRSSQSLVHTNGNTYLHWYLQKPGQSPKLLIYKVSNRFSGVPDRFSGSGSGTDFTLKISRVEAEDLGVYFCSQTTHVPLTFGAGTKLELK

SEQ ID NO: 68 - protein, 124 aa, Mus musculus
QVTLKESGPGILQPSQTLSLTCSFSGFSLSTFGRGVGWIRQPSGKGLDWLTHIWWDDDKYYNPALKSRLTISKDTSKNQVFLKIANVDTADTATYYCARIAGGLRRAPYAMDSWGQGTSVTVSS

SEQ ID NO: 69 - protein, 107 aa, Mus musculus
DIQMTQSPASLSVSVGETVTITCRASENIYSSLAWYQQKQGKSPQLLVYAATNLADGVPSRFSGSGSGTQYSLKINSLQSEDSGNYYCQHFRDTPPTFGGGTKLEIK

SEQ ID NO: 70 - protein, 115 aa, Mus musculus
EVQLVESGGDLVKPGGSLKLSCAASGFTFSSYGMSWVRQTPDKRLEWVATISNGGSYTYYPDSVKGRFTISRDNAKNTLYMQMSSLKSEDTAMYYCARNEPPDYWGQGTSVTVSS

SEQ ID NO: 71 - protein, 108 aa, Mus musculus
QIVLTQSPAIMSASLGERVTMTCTASSSVGSSYLHWYQQKPGSSPKLWIYDTSNLASGVPVRFSGSGSGTSYSLTISSMEAEDAATYYCHQYHRSPYTFGGGTKLEIK

SEQ ID NO: 72 - protein, 454 aa, synthetic construct
  REGION 1..454: note = caninized mouse
EVQLVQSGAEVKKPGASVKVSCKTSGYTFINTFIHWVRQAPGAGLDWMGQIDPANGNTKYAPKFQGRVTLTADTSTSTAYMELSSLRAGDIAVYYCARYYYVSSYFDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 73 - protein, 454 aa, synthetic construct
  REGION 1..454: note = caninized mouse
EVQLVQSGAEVKKPGASVKVSCTASGFNIKNTFIHWVRQAPGAGLDWIGRIDPANGNTKYAPKFQGRVTLTADTSTSTAYMELSSLRAGDIAVYYCARYYYVSSYFDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 74 - protein, 454 aa, synthetic construct
  REGION 1..454: note = caninized mouse
EVQLVQSGAEVKKPGASVKVSCTASGFNIKNTFIHWVRQAPGAGLDWIGRIDPANGNTKYAPKFQGRATLTADTSTNTAYMQLSSLRAGDIAVYYCARYYYVSSYFDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 75 - protein, 564 aa, synthetic construct
  REGION 1..564: note = caninized mouse
EVQLVQSGAEVVKPGASVKVSCTASGFNIKNTFIHWVRQRPGAGLDWIGRIDPANGNTKYAPKFQGRATLTADTSTNTAYMQLSSLRAGDIAVYYCARYYYVSSYFDVWGTGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 76 - protein, 217 aa, synthetic construct
  REGION 1..217: note = caninized mouse
EIVMTQSPASLSLSQEEKVTITCKASQSVTNDVTWYQQKPGQAPKLLIYSASNRYTGVPSRFSGSGSGTDFSFTISSLEPEDVAVYYCQQDYSSPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 77 - protein, 217 aa, synthetic construct
  REGION 1..217: note = caninized mouse
DIVMTQTPLSLSVSPGETASISCKASQSVTNDVTWFRQKPGQSPQLLIYSASNRYTGVPDRFSGSGSGTDFTLRISTVEADDTGVYYCQQDYSSPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 78 - protein, 217 aa, synthetic construct
  REGION 1..217: note = caninized mouse
DIVMTQTPLSLSVSPGETASISCKASQSVTNDVTWFRQKPGQSPKLLIYSASNRYTGVPDRFSGSGSGTDFTLRISTVEADDTGVYYCQQDYSSPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 79 - protein, 458 aa, synthetic construct
  REGION 1..458: note = caninized mouse
EVQLVQSGAEVKKPGASVKVSCKTSGYTFTDYNMDWVRQAPGAGLDWMGHINPNNGGILYNQKFKGRVTLTADTSTSTAYMELSSLRAGDIAVYYCARWAPLVRQPYWYFDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 80 - protein, 458 aa, synthetic construct
  REGION 1..458: note = caninized mouse
EVQLVQSGAEVKKPGASVKVSCKASGYTFTDYNMDWVRQAPGAGLDWIGHINPNNGGILYNQKFKGKATLTADTSTSTAYMELSSLRAGDIAVYYCARWAPLVRQPYWYFDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 81 - protein, 458 aa, synthetic construct
  REGION 1..458: note = caninized mouse
EVQLVQSGAEVKKPGASVKVSCKASGYTFTDYNMDWVRQAPGAGLDWIGHINPNNGGILYNQKFKGKATLTVDRSTNTAYMELSSLRAGDIAVYFCARWAPLVRQPYWYFDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 82 - protein, 217 aa, synthetic construct
  REGION 1..217: note = caninized mouse
EIVMTQSPASLSLSQEEKVTITCKASQDINSYLNWYQQKPGQAPKLLIYRADRLVDGVPSRFSGSGSGTDFSFTISSLEPEDVAVYYCLQYDEFPLTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 83 - protein, 217 aa, synthetic construct
  REGION 1..217: note = caninized mouse
EIVMTQSPASLSLSQEEKVTITCKASQDINSYLNWFQQKPGQAPKLLIYRADRLVDGVPSRFSGSGSGTDFSFTISSLEPEDVAVYYCLQYDEFPLTFGAGTKLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 84 - protein, 217 aa, synthetic construct
  REGION 1..217: note = caninized mouse
DIKMTQSPASLSLSQEEKVTITCKASQDINSYLNWFQQKPGQAPKLLIYRADRLVDGVPSRFSGSGSGTDFSFTISSLEPEDVAVYYCLQYDEFPLTFGAGTKLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 85 - protein, 217 aa, synthetic construct
  REGION 1..217: note = caninized mouse
DIVMTQTPLSLSVSPGETASISCKASQDINSYLNWFRQKPGQSPQLLIYRADRLVDGVPDRFSGSGSGTDFTLRISTVEADDTGVYYCLQYDEFPLTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 86 - protein, 459 aa, synthetic construct
  REGION 1..459: note = caninized mouse
ELTLQESGPGLVKPSQTLSLTCVVSGGSVTTFGRGVGWIRQRPGRGLEWMGHIWWDDDKYYNPALKSRISITADTAKNQFSLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 87 - protein, 459 aa, synthetic construct
  REGION 1..459: note = caninized mouse
ELTLQESGPGLVKPSQTLSLTCSFSGFSLSTFGRGVGWIRQRPGRGLEWMGHIWWDDDKYYNPALKSRISITADTAKNQFSLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 88 - protein, 459 aa, synthetic construct
  REGION 1..459: note = caninized mouse
EVTLQESGPGLVKPSQTLSLTCSFSGFSLSTFGRGVGWIRQRPGRGLEWMGHIWWDDDKYYNPALKSRLSITKDTAKNQFSLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 89 - protein, 459 aa, synthetic construct
  REGION 1..459: note = caninized mouse
EVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 90 - protein, 217 aa, synthetic construct
  REGION 1..217: note = caninized mouse
EIVMTQSPASLSLSQEEKVTITCRASENIYSSLAWYQQKPGQAPKLLIYAATNLADGVPSRFSGSGSGTDFSFTISSLEPEDVAVYYCQHFRDTPPTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 91 - protein, 217 aa, synthetic construct
  REGION 1..217: note = caninized mouse
DIVMTQTPLSLSVSPGETASISCRASENIYSSLAWFRQKPGQSPQLLIYAATNLADGVPDRFSGSGSGTDFTLRISRVEADDTGVYYCQHFRDTPPTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 92 - protein, 217 aa, synthetic construct
  REGION 1..217: note = caninized mouse
DIVMTQTPLSLSVSPGETASISCRASENIYSSLAWFRQKPGQSPQLLVYAATNLADGVPDRFSGSGSGTDYTLRISRVEADDTGVYYCQHFRDTPPTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 93 - protein, 124 aa, synthetic construct
  REGION 1..124: note = caninized mouse
ELTLQESGPGLVKPSQTLSLTCVVSGGSVTTFGRGVGWIRQRPGRGLEWMGHIWWDDDKYYNPALKSRISITADTAKNQFSLQLSSMTTEDTAVYYCARIAGGLRRAPYAMDSWGQGTLVTVSS

SEQ ID NO: 94 - protein, 124 aa, synthetic construct
  REGION 1..124: note = caninized mouse
ELTLQESGPGLVKPSQTLSLTCSFSGFSLSTFGRGVGWIRQRPGRGLEWMGHIWWDDDKYYNPALKSRISITADTAKNQFSLQLSSMTTEDTAVYYCARIAGGLRRAPYAMDSWGQGTLVTVSS

SEQ ID NO: 95 - protein, 124 aa, synthetic construct
  REGION 1..124: note = caninized mouse
EVTLQESGPGLVKPSQTLSLTCSFSGFSLSTFGRGVGWIRQRPGRGLEWMGHIWWDDDKYYNPALKSRLSITKDTAKNQFSLQLSSMTTEDTAVYYCARIAGGLRRAPYAMDSWGQGTLVTVSS

SEQ ID NO: 96 - protein, 124 aa, synthetic construct
  REGION 1..124: note = caninized mouse
EVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSS

SEQ ID NO: 97 - protein, 19 aa, Canis lupus
  source 1..19: note = Canis familiaris
YWNLDAIMKIEPPEIFSVK

SEQ ID NO: 98 - protein, 18 aa, Canis lupus
  source 1..18: note = Canis familiaris
KIEPPEIFSVKSVLGIKR

SEQ ID NO: 99 - protein, 13 aa, Canis lupus
  source 1..13: note = Canis familiaris
RPVLAPHSSTLKY

SEQ ID NO: 100 - protein, 14 aa, Canis lupus
  source 1..14: note = Canis familiaris
TEYVMTLRCAPAES

SEQ ID NO: 101 - protein, 6 aa, Canis lupus
  source 1..6: note = Canis familiaris
YVMTLR

SEQ ID NO: 102 - protein, 8 aa, Canis lupus
  source 1..8: note = Canis familiaris
RCAPAESM

SEQ ID NO: 103 - protein, 20 aa, Canis lupus
  source 1..20: note = Canis familiaris
RCAPAESMFWSGWSQEKVGT

SEQ ID NO: 104 - protein, 12 aa, Canis lupus
  source 1..12: note = Canis familiaris
RRPVQLMWKKAT

SEQ ID NO: 105 - protein, 9 aa, Canis lupus
  source 1..9: note = Canis familiaris
RIPALNEKT

SEQ ID NO: 106 - protein, 12 aa, Canis lupus
  source 1..12: note = Canis familiaris
RNGFIKNYTIFY

SEQ ID NO: 107 - protein, 17 aa, Canis lupus
  source 1..17: note = Canis familiaris
RTSYSLQVMASTNAGGT

SEQ ID NO: 108 - protein, 16 aa, Canis lupus
  source 1..16: note = Canis familiaris
SLQVMASTNAGGTNGT

SEQ ID NO: 109 - protein, 10 aa, Canis lupus
  source 1..10: note = Canis familiaris
STNAGGTNGT

SEQ ID NO: 110 - protein, 215 aa, Canis lupus
  source 1..215: note = Canis familiaris
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 111 - protein, 214 aa, synthetic construct
  REGION 1..214: note = modified canine
LGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 112 - protein, 17 aa, Canis lupus
  source 1..17: note = Canis familiaris
FNECRCTDTPPCPVPEP

SEQ ID NO: 113 - protein, 22 aa, Canis lupus
  source 1..22: note = Canis familiaris
PKRENGRVPRPPDCPKCPAPEM

SEQ ID NO: 114 - protein, 20 aa, Canis lupus
  source 1..20: note = Canis familiaris
AKECECKCNCNNCPCPGCGL

SEQ ID NO: 115 - protein, 17 aa, synthetic construct
  REGION 1..17: note = modified canine
PKESTCKCIPPCPVPES

SEQ ID NO: 116 - protein, 216 aa, Canis lupus
  source 1..216: note = Canis familiaris
LGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 117 - protein, 215 aa, Canis lupus
  source 1..215: note = Canis familiaris
LGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 118 - protein, 216 aa, Canis lupus
  source 1..216: note = Canis familiaris
LGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 119 - protein, 31 aa, Canis lupus
  source 1..31: note = Canis familiaris
SDITYWNLDAIMKIEPPEIFSVKSVLGIKRM

SEQ ID NO: 120 - protein, 31 aa, Canis lupus
  source 1..31: note = Canis familiaris
MTLRCAPAESMFWSGWSQEKVGTTEEEAPYG

SEQ ID NO: 121 - protein, 21 aa, Canis lupus
  source 1..21: note = Canis familiaris
IMKIEPPEIFSVKSVLGIKRM

SEQ ID NO: 122 - protein, 21 aa, Canis lupus
  source 1..21: note = Canis familiaris
TELQAFTEYVMTLRCAPAESM

SEQ ID NO: 123 - protein, 21 aa, Canis lupus
  source 1..21: note = Canis familiaris
AMVDGRRPVQLMWKKATGAPV

SEQ ID NO: 124 - protein, 21 aa, Canis lupus
  source 1..21: note = Canis familiaris
GESPVATLRIPALNEKTFQCI

SEQ ID NO: 125 - protein, 21 aa, Canis lupus
  source 1..21: note = Canis familiaris
RNGFIKNYTIFYQAEDGKEFS

SEQ ID NO: 126 - protein, 21 aa, Canis lupus
  source 1..21: note = Canis familiaris
SYSLQVMASTNAGGTNGTKIN

SEQ ID NO: 127 - protein, 110 aa, synthetic construct
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 128 - protein, 107 aa, synthetic construct
EIVMTQSPASLSLSQEEKVTITCRASENIYSSLAWYQQKPGQAPKLLIYAATNLADGVPSRFSGSGSGTDFSFTISSLEPEDVAVYYCQHFRDTPPTFGQGTKLEIK

SEQ ID NO: 129 - protein, 107 aa, synthetic construct
DIVMTQTPLSLSVSPGETASISCRASENIYSSLAWFRQKPGQSPQLLIYAATNLADGVPDRFSGSGSGTDFTLRISRVEADDTGVYYCQHFRDTPPTFGQGTKLEIK

SEQ ID NO: 130 - protein, 107 aa, synthetic construct
DIVMTQTPLSLSVSPGETASISCRASENIYSSLAWFRQKPGQSPQLLVYAATNLADGVPDRFSGSGSGTDYTLRISRVEADDTGVYYCQHFRDTPPTFGQGTKLEIK

(19) United States
(12) Patent Application Publication
(10) Pub. No.: US 2025/0340653 A1
(43) Pub. Date: Nov. 6, 2025

(54) CANINIZED ANTIBODIES TO CANINE INTERLEUKIN-31 RECEPTOR ALPHA I

(71) Applicant: INTERVET INC., Madison, NJ (US)

(72) Inventors: Mohamad MORSEY, Omaha, NE (US); Yuanzheng ZHANG, Somerset, NJ (US)

(73) Assignee: INTERVET INC., Madison, NJ (US)

(21) Appl. No.: 18/720,564

(22) PCT Filed: Dec. 15, 2022
(86) PCT No.: PCT/EP2022/086084

§ 371 Date: Jun. 14, 2024

Related U.S. Application Data
(60) Provisional application No. 63/290,259, filed on Dec. 16, 2021.
(60) Provisional application No. 63/290,256, filed on Dec. 16, 2021.
(60) Provisional application No. 63/341,443, filed on May 13, 2022.

(51) Int. Cl. C07K 16/28

(52) U.S. Cl. CPC C07K 16/2866; C07K 2317/24; C07K 2317/76; C07K 2317/92

(57) ABSTRACT

The present invention provides caninized mouse antibodies to canine IL-31 receptor alpha that have a high binding affinity for canine IL-31 receptor alpha, and that can block the binding of canine IL-31 to canine IL-31 receptor alpha. The present invention further provides the use of the antibodies for the treatment of atopic dermatitis in dogs.

### REFERENCE TO SEQUENCE LISTING SUBMITTED ELECTRONICALLY

This application contains an electronic Substitute Sequence Listing which has been submitted in XML format via Patent Center, the entire content of which is incorporated by reference herein in its entirety. The Substitute Sequence Listing XML file submitted via Patent Center is entitled “14463-298-999_SUB SEQ LISTING.xml”, was created on Jan. 2, 2025, and is 142,308 bytes in size.

### CROSS-REFERENCE TO RELATED APPLICATIONS

This application is a U.S. National Stage Application under 35 U.S.C. § 371 of International Patent Application No. PCT/EP2022/086084, filed on Dec. 15, 2022, which claims the benefit of U.S. Provisional Application No. 63/341,443, filed on May 13, 2022, U.S. Provisional Application No. 63/290,259, filed on Dec. 16, 2021, and U.S. Provisional Application No. 63/290,256, filed on Dec. 16, 2021, the disclosure of each of which is incorporated by reference herein in its entirety.

### FIELD OF THE INVENTION

The present invention relates to antibodies to canine IL-31 receptor alpha that have a high binding affinity for canine IL-31 receptor alpha, and that can block the binding of canine IL-31 to the canine IL-31 receptor alpha. The present invention also relates to use of the antibodies of the present invention in the treatment of atopic dermatitis in dogs.

### BACKGROUND OF THE INVENTION

The immune system comprises a network of resident and recirculating specialized cells that function collaboratively to protect the host against infectious diseases and cancer. The ability of the immune system to perform this function depends to a large extent on the biological activities of a group of proteins secreted by leukocytes and collectively referred to as interleukins. Among the well-studied interleukins are four important molecules identified as interleukin-31 (IL-31), interleukin-4 (IL-4), interleukin-13 (IL-13), and interleukin-22 (IL-22). Although IL-4, IL-13, IL-22, and IL-31, are critical cytokines for the development of immune responses that are required for protection against extracellular pathogens (e.g., tissue or lumen dwelling parasites), these cytokines also have been implicated in the pathogenesis of allergic diseases in humans and animals, including atopic dermatitis.

Atopic dermatitis (AD) is a relapsing pruritic and chronic inflammatory skin disease, that is characterized by immune system dysregulation and epidermal barrier abnormalities in humans. The pathological and immunological attributes of atopic dermatitis have been the subject of extensive investigations [reviewed in Rahman et al. Inflammation & Allergy-drug target 10:486-496 (2011) and Harskamp et al., Seminar in Cutaneous Medicine and Surgery 32:132-139 (2013)]. Atopic dermatitis is also a common condition in companion animals, especially dogs, where its prevalence has been estimated to be approximately 10-15% of the canine population. The pathogenesis of atopic dermatitis in dogs and cats [reviewed in Nuttall et al., Veterinary Records 172(8):201-207 (2013)] shows significant similarities to that of atopic dermatitis in man including skin infiltration by a variety of immune cells and CD4⁺ Th2 polarized cytokine milieu including the preponderance of IL-31, IL-4, and IL-13. In addition, IL-22 has been implicated in the exaggerated epithelial proliferation leading to epidermal hyperplasia that is characteristic of atopic dermatitis.

For example, antibodies against canine IL-31 have been shown to have an effect on pruritus associated with atopic dermatitis in dogs [U.S. Pat. No. 8,790,651 B2; U.S. Pat. No. 10,093,731 B2]. In addition, an antibody against human IL-31 receptor alpha (IL-31RA) has been tested and found to have an effect on pruritus associated with atopic dermatitis in humans [Ruzicka, et al., New England Journal of Medicine, 376(9), 826-835 (2017)].

Pharmaceuticals that have either proven to aid in the treatment of atopic dermatitis and/or have shown promise to do so include: Janus kinase (JAK) inhibitors [see e.g., U.S. Pat. Nos. 8,133,899; 8,987,283; WO 2018/108969], spleen tyrosine kinase (SYK) inhibitors [see e.g., U.S. Pat. No. 8,759,366], and antagonists to a chemoattractant receptor-homologous molecule expressed on TH2 cells [see e.g., U.S. Pat. Nos. 7,696,222, 8,546,422, 8,637,541, and 8,546,422].

However, despite some recent success in treating atopic dermatitis, there remains a need to design alternative and/or better therapies that can address one or more of the symptoms of canine atopic dermatitis.

The citation of any reference herein should not be construed as an admission that such reference is available as “prior art” to the instant application.

### SUMMARY OF THE INVENTION

The present invention provides new mammalian antibodies, including caninized murine antibodies, to IL-31 receptor alpha (TL-3IRA) from canines. In certain embodiments, the mammalian antibodies to canine IL-31 receptor alpha (cIL-31RA) are isolated antibodies. In preferred embodiments, the mammalian antibodies or antigen binding fragments thereof bind canine IL-31RA. In more particular embodiments, the mammalian antibodies or antigen binding fragments also block the binding of canine IL-31RA to canine interleukin-31. In certain embodiments, the mammalian antibodies are antibodies to canine IL-31RA. In more particular embodiments, the mammalian antibodies are caninized antibodies. In even more particular embodiments, the caninized antibodies are caninized murine antibodies to canine IL-31RA.

Accordingly, the present invention provides mammalian antibodies (including caninized antibodies) or antigen binding fragments thereof that bind canine IL-31RA, in which the antibody comprises a heavy chain and a light chain that together comprise a set of six complementary determining regions (CDRs), three of which are heavy chain CDRs: CDR heavy 1 (HCDR1), CDR heavy 2 (HCDR2) and CDR heavy 3 (HCDR3) and three of which are light chain CDRs: CDR light 1 (LCDR1), CDR light 2 (LCDR2), and CDR light 3 (LCDR3).

In specific embodiments, the mammalian antibody or antigen binding fragment thereof comprises an HCDR1 that comprises the amino acid sequence of SEQ ID NO: 4, an HCDR2 that comprises the amino acid sequence of SEQ ID NO: 14, and an HCDR3 that comprises the amino acid sequence of SEQ ID NO: 24. In particular embodiments of this type, the mammalian antibody or antigen binding fragment thereof further comprises a LCDR1 that comprises the amino acid sequence of SEQ ID NO: 33, a LCDR2 that comprises the amino acid sequence of SEQ ID NO: 37, and the LCDR3 that comprises the amino acid sequence of SEQ ID NO: 46. In more particular embodiments, the mammalian antibody or antigen binding fragment thereof when bound to canine IL-31RA binds to an epitope comprised by the amino acid sequence of SEQ ID NO: 106. In other embodiments, the mammalian antibody or antigen binding fragment thereof when bound to canine IL-31RA binds to an epitope comprised by an amino acid sequence of SEQ ID NO: 108. In still other embodiments, the mammalian antibody or antigen binding fragment thereof when bound to canine IL-31RA binds both to an epitope comprised by the amino acid sequence of SEQ ID NO: 106 and the amino acid sequence of SEQ ID NO: 108.

In alternative embodiments, the mammalian antibody or antigen binding fragment thereof comprises an HCDR1 that comprises the amino acid sequence of SEQ ID NO: 5, an HCDR2 that comprises the amino acid sequence of SEQ ID NO: 15, and an HCDR3 that comprises the amino acid sequence of SEQ ID NO: 25. In particular embodiments of this type, the mammalian antibody or antigen binding fragment thereof further comprises a LCDR1 that comprises the amino acid sequence of SEQ ID NO: 32, a LCDR2 that comprises the amino acid sequence of SEQ ID NO: 41, and the LCDR3 that comprises the amino acid sequence of SEQ ID NO: 47. In more particular embodiments, the mammalian antibody or antigen binding fragment thereof when bound to canine IL-31RA binds to an epitope comprised by the amino acid sequence of SEQ ID NO: 98. In other embodiments, the mammalian antibody or antigen binding fragment thereof when bound to canine IL-31RA binds to an epitope comprised by an amino acid sequence of SEQ ID NO: 100. In still other embodiments, the mammalian antibody or antigen binding fragment thereof when bound to canine IL-31RA binds both to an epitope comprised by the amino acid sequence of SEQ ID NO: 98 and the amino acid sequence of SEQ ID NO: 100.

In other embodiments, the mammalian antibody or antigen binding fragment thereof comprises an HCDR1 that comprises the amino acid sequence of SEQ ID NO: 6, an HCDR2 that comprises the amino acid sequence of SEQ ID NO: 16, and an HCDR3 that comprises the amino acid sequence of SEQ ID NO: 26. In particular embodiments of this type, the mammalian antibody or antigen binding fragment thereof further comprises a LCDR1 that comprises the amino acid sequence of SEQ ID NO: 32, a LCDR2 that comprises the amino acid sequence of SEQ ID NO: 42, and the LCDR3 that comprises the amino acid sequence of SEQ ID NO: 48. In more particular embodiments, the mammalian antibody or antigen binding fragment thereof when bound to canine IL-31RA binds to an epitope comprised by the amino acid sequence of SEQ ID NO: 98. In other embodiments, the mammalian antibody or antigen binding fragment thereof when bound to canine IL-31RA binds to an epitope comprised by an amino acid sequence of SEQ ID NO: 101. In still other embodiments, the mammalian antibody or antigen binding fragment thereof when bound to canine IL-31RA binds both to an epitope comprised by the amino acid sequence of SEQ ID NO: 98 and the amino acid sequence of SEQ ID NO: 101.

In still other embodiments, the mammalian antibody or antigen binding fragment thereof comprises an HCDR1 that comprises the amino acid sequence of SEQ ID NO: 7, an HCDR2 that comprises the amino acid sequence of SEQ ID NO: 17, and an HCDR3 that comprises the amino acid sequence of SEQ ID NO: 27. In particular embodiments of this type, the mammalian antibody or antigen binding fragment thereof further comprises a LCDR1 that comprises the amino acid sequence of SEQ ID NO: 34, a LCDR2 that comprises the amino acid sequence of SEQ ID NO: 37, and the LCDR3 that comprises the amino acid sequence of SEQ ID NO: 50. In more particular embodiments, the mammalian antibody or antigen binding fragment thereof when bound to canine IL-31RA binds to an epitope comprised by the amino acid sequence of SEQ ID NO: 106. In other embodiments, the mammalian antibody or antigen binding fragment thereof when bound to canine IL-31RA binds to an epitope comprised by an amino acid sequence of SEQ ID NO: 107. In still other embodiments, the mammalian antibody or antigen binding fragment thereof when bound to canine IL-31RA binds both to an epitope comprised by the amino acid sequence of SEQ ID NO: 106 and the amino acid sequence of SEQ ID NO: 107.

In yet other embodiments, the mammalian antibody or antigen binding fragment thereof comprises an HCDR1 that comprises the amino acid sequence of SEQ ID NO: 8, an HCDR2 that comprises the amino acid sequence of SEQ ID NO: 18, and an HCDR3 that comprises the amino acid sequence of SEQ ID NO: 28. In particular embodiments of this type, the mammalian antibody or antigen binding fragment thereof further comprises a LCDR1 that comprises the amino acid sequence of SEQ ID NO: 35, a LCDR2 that comprises the amino acid sequence of SEQ ID NO: 43, and the LCDR3 that comprises the amino acid sequence of SEQ ID NO: 51. In more particular embodiments, the mammalian antibody or antigen binding fragment thereof when bound to canine IL-31RA binds to an epitope comprised by the amino acid sequence of SEQ ID NO: 104. In other embodiments, the mammalian antibody or antigen binding fragment thereof when bound to canine IL-31RA binds to an epitope comprised by an amino acid sequence of SEQ ID NO: 105. In still other embodiments, the mammalian antibody or antigen binding fragment thereof when bound to canine IL-31RA binds both to an epitope comprised by the amino acid sequence of SEQ ID NO: 104 and the amino acid sequence of SEQ ID NO: 105.

In still other embodiments, the mammalian antibody or antigen binding fragment thereof comprises an HCDR1 that comprises the amino acid sequence of SEQ ID NO: 9, an HCDR2 that comprises the amino acid sequence of SEQ ID NO: 19, and an HCDR3 that comprises the amino acid sequence of SEQ ID NO: 29. In particular embodiments of this type, the mammalian antibody or antigen binding fragment thereof further comprises a LCDR1 that comprises the amino acid sequence of SEQ ID NO: 36, a LCDR2 that comprises the amino acid sequence of SEQ ID NO: 44, and the LCDR3 that comprises the amino acid sequence of SEQ ID NO: 49. In more particular embodiments, the mammalian antibody or antigen binding fragment thereof when bound to canine IL-31RA binds to an epitope comprised by the amino acid sequence of SEQ ID NO: 109.

In preferred embodiments, the antibody and antigen binding fragment thereof bind canine IL-31RA and block the binding of canine IL-31RA to canine interleukin-31. In specific embodiments, the mammalian antibody to canine IL-31RA is a murine antibody. In particular embodiments, the mammalian antibody to canine TL-3IRA is a caninized antibody. In more particular embodiments, the caninized antibody to canine IL-31RA is a caninized murine antibody.

The caninized antibodies of the present invention comprise a canine fragment crystallizable region (cFc). The caninized antibodies of the present invention also comprise a canine light chain constant region. In particular embodiments the canine light chain constant region is a kappa canine light chain constant region. In more specific embodiments, the kappa canine light chain constant region comprises the amino acid sequence of SEQ ID NO: 127.

Furthermore the caninized antibody or antigen binding fragment thereof can comprise a heavy chain that comprises a cFc region and a hinge region. The hinge region is preferably a canine hinge region. The canine hinge region can comprise a natural occurring: IgG-A hinge region, IgG-B hinge region, IgG-C hinge region, or IgG-D hinge region. Alternatively, the hinge region is a corresponding modified canine hinge region. In particular embodiments, the hinge region is the IgG-A hinge region comprising an amino acid sequence comprising at least 90%, 95%, or 100% identity with the amino acid sequence of SEQ ID NO: 112. In other embodiments, the hinge region is the IgG-B hinge region comprising an amino acid sequence comprising at least 90%, 95%, or 100% identity with the amino acid sequence of SEQ ID NO: 113. In still other embodiments, the hinge region is the IgG-C hinge region comprising an amino acid sequence comprising at least 90%, 95%, or 100% identity with the amino acid sequence of SEQ ID NO: 114. In yet other embodiments, the hinge region is a modified IgG-D hinge region comprising the amino acid sequence of SEQ ID NO: 115.

Similarly, the canine Fc region can be an IgG-A, IgG-B, IgG-C, an IgG-D or modifications thereof. In particular embodiments, a caninized antibody or antigen binding fragment thereof comprises an IgG-Bm. In certain embodiments, a caninized antibody or antigen binding fragment thereof comprises an IgG-A that comprises an amino acid sequence that has at least 90%, 95%, 98%, 99% or 100% identity with the amino acid sequence of SEQ ID NO: 116. In other embodiments, a caninized antibody or antigen binding fragment thereof comprises an IgG-B that comprises an amino acid sequence that has at least 90%, 95%, 98%, 99%, or 100% identity with the amino acid sequence of SEQ ID NO: 110. In still other embodiments, a caninized antibody or antigen binding fragment thereof comprises an IgG-C that comprises an amino acid sequence that has at least 90%, 95%, 98%, 99%, or 100% identity with the amino acid sequence of SEQ ID NO: 117. In yet other embodiments, a caninized antibody or antigen binding fragment thereof comprises an IgG-D that comprises an amino acid sequence that has at least 90%, 95%, 98%, 99% or 100% identity with the amino acid sequence of SEQ ID NO: 118. In still other embodiments, a caninized antibody or antigen binding fragment thereof comprises an IgG-Bm that comprises an amino acid sequence that has at least 90%, 95%, 98%, 99%, or 100% identity with the amino acid sequence of SEQ ID NO: 111, wherein both the aspartic acid residue (D) at position 31 of SEQ ID NO: 110 and the asparagine residue (N) at position 63 of SEQ ID NO: 110, remain substituted by an alanine residue (A) in the sequence of IgG-Bm.

In particular embodiments, the caninized antibody or antigen binding fragment thereof comprises the canine IgG-D, but the naturally occurring IgG-D hinge region is replaced by a hinge region comprising an amino acid sequence comprising at least 90%, 95%, or 100% identity with the amino acid sequence of SEQ ID NO: 112. In other embodiments, the caninized antibody comprises a heavy chain that comprises an IgG-D, but the naturally occurring IgG-D hinge region is replaced by a hinge region comprising an amino acid sequence comprising at least 90%, 95%, or 100% identity with the amino acid sequence of SEQ ID NO: 113. In still other embodiments, the caninized antibody comprises a heavy chain that comprises an IgG-D, but the naturally occurring IgG-D hinge region is replaced by a hinge region comprising an amino acid sequence comprising at least 90%, 95%, or 100% identity with the amino acid sequence of SEQ ID NO: 114. In yet other embodiments, the caninized antibody comprises a heavy chain that comprises an IgG-D, but the naturally occurring IgG-D hinge region is replaced by a hinge region comprising the amino acid sequence of SEQ ID NO: 115.

In certain embodiments, the caninized antibody against canine TL-3IRA or antigen binding fragment thereof comprises a heavy chain variable region comprising the amino acid sequence of SEQ ID NO: 93. In other embodiments, the caninized antibody against canine IL-31RA or antigen binding fragment thereof comprises a heavy chain variable region comprising the amino acid sequence of SEQ ID NO: 94. In yet other embodiments, the caninized antibody against canine TL-3IRA or antigen binding fragment thereof comprises a heavy chain variable region comprising the amino acid sequence of SEQ ID NO: 95. In still other embodiments, the caninized antibody against canine TL-3IRA or antigen binding fragment thereof comprises a heavy chain variable region comprising the amino acid sequence of SEQ ID NO: 96. All of these heavy chain variable regions can further comprise a canine hinge region and/or a cFc that have been aforementioned above.

In related embodiments, the caninized antibody against canine IL-31RA or antigen binding fragment thereof comprises a light chain variable region comprising the amino acid sequence of SEQ ID NO: 128. In other embodiments, the caninized antibody against canine IL-31RA or antigen binding fragment thereof comprises a light chain variable region comprising the amino acid sequence of SEQ ID NO: 129. In still other embodiments, the caninized antibody against canine IL-31RA or antigen binding fragment thereof comprises a light chain variable region comprising the amino acid sequence of SEQ ID NO: 130.

In yet other embodiments, the caninized antibody comprises a heavy chain variable region comprising the amino acid sequence of SEQ ID NO: 93 and a light chain variable region comprising the amino acid sequence of SEQ ID NO: 128. In still other embodiments, the caninized antibody comprises a heavy chain variable region comprising the amino acid sequence of SEQ ID NO: 93 and a light chain variable region comprising the amino acid sequence of SEQ ID NO: 129. In yet other embodiments, the caninized antibody comprises a heavy chain variable region comprising the amino acid sequence of SEQ ID NO: 93 and a light chain variable region comprising the amino acid sequence of SEQ ID NO: 130.

In still other embodiments, the caninized antibody comprises a heavy chain variable region comprising the amino acid sequence of SEQ ID NO: 94 and a light chain variable region comprising the amino acid sequence of SEQ ID NO: 128. In yet other embodiments, the caninized antibody comprises a heavy chain variable region comprising the amino acid sequence of SEQ ID NO: 94 and a light chain variable region comprising the amino acid sequence of SEQ ID NO: 129. In still other embodiments, the caninized antibody comprises a heavy chain variable region comprising the amino acid sequence of SEQ ID NO: 94 and a light chain variable region comprising the amino acid sequence of SEQ ID NO: 130.

In yet other embodiments, the caninized antibody comprises a heavy chain variable region comprising the amino acid sequence of SEQ ID NO: 95 and a light chain variable region comprising the amino acid sequence of SEQ ID NO: 128. In still other embodiments, the caninized antibody comprises a heavy chain variable region comprising the amino acid sequence of SEQ ID NO: 95 and a light chain variable region comprising the amino acid sequence of SEQ ID NO: 129. In yet other embodiments, the caninized antibody comprises a heavy chain variable region comprising the amino acid sequence of SEQ ID NO: 95 and a light chain variable region comprising the amino acid sequence of SEQ ID NO: 130.

In still other embodiments, the caninized antibody comprises a heavy chain variable region comprising the amino acid sequence of SEQ ID NO: 96 and a light chain variable region comprising the amino acid sequence of SEQ ID NO: 128. In yet other embodiments, the caninized antibody comprises a heavy chain variable region comprising the amino acid sequence of SEQ ID NO: 96 and a light chain variable region comprising the amino acid sequence of SEQ ID NO: 129. In still other embodiments, the caninized antibody comprises a heavy chain variable region comprising the amino acid sequence of SEQ ID NO: 96 and a light chain variable region comprising the amino acid sequence of SEQ ID NO: 130.

In related embodiments, the caninized antibody against canine TL-3IRA or antigen binding fragment thereof comprises a light chain comprising the amino acid sequence of SEQ ID NO: 90. In other embodiments, the caninized antibody against canine TL-3IRA or antigen binding fragment thereof comprises a light chain comprising the amino acid sequence of SEQ ID NO: 91. In still other embodiments, the caninized antibody against canine IL-31RA or antigen binding fragment thereof comprises a light chain comprising the amino acid sequence of SEQ ID NO: 92.

In yet other embodiments, the caninized antibody comprises a heavy chain variable region comprising the amino acid sequence of SEQ ID NO: 93 and a light chain comprising the amino acid sequence of SEQ ID NO: 90. In still other embodiments, the caninized antibody comprises a heavy chain variable region comprising the amino acid sequence of SEQ ID NO: 93 and a light chain comprising the amino acid sequence of SEQ ID NO: 91. In yet other embodiments, the caninized antibody comprises a heavy chain variable region comprising the amino acid sequence of SEQ ID NO: 93 and a light chain comprising the amino acid sequence of SEQ ID NO: 92.

In still other embodiments, the caninized antibody comprises a heavy chain variable region comprising the amino acid sequence of SEQ ID NO: 94 and a light chain comprising the amino acid sequence of SEQ ID NO: 90. In yet other embodiments, the caninized antibody comprises a heavy chain variable region comprising the amino acid sequence of SEQ ID NO: 94 and a light chain comprising the amino acid sequence of SEQ ID NO: 91. In still other embodiments, the caninized antibody comprises a heavy chain variable region comprising the amino acid sequence of SEQ ID NO: 94 and a light chain comprising the amino acid sequence of SEQ ID NO: 92.

In yet other embodiments, the caninized antibody comprises a heavy chain variable region comprising the amino acid sequence of SEQ ID NO: 95 and a light chain comprising the amino acid sequence of SEQ ID NO: 90. In still other embodiments, the caninized antibody comprises a heavy chain variable region comprising the amino acid sequence of SEQ ID NO: 95 and a light chain comprising the amino acid sequence of SEQ ID NO: 91. In yet other embodiments, the caninized antibody comprises a heavy chain variable region comprising the amino acid sequence of SEQ ID NO: 95 and a light chain comprising the amino acid sequence of SEQ ID NO: 92.

In still other embodiments, the caninized antibody comprises a heavy chain variable region comprising the amino acid sequence of SEQ ID NO: 96 and a light chain comprising the amino acid sequence of SEQ ID NO: 90. In yet other embodiments, the caninized antibody comprises a heavy chain variable region comprising the amino acid sequence of SEQ ID NO: 96 and a light chain comprising the amino acid sequence of SEQ ID NO: 91. In still other embodiments, the caninized antibody comprises a heavy chain variable region comprising the amino acid sequence of SEQ ID NO: 96 and a light chain comprising the amino acid sequence of SEQ ID NO: 92.

In certain embodiments, the caninized antibody against canine IL-31RA or antigen binding fragment thereof comprises a heavy chain comprising the amino acid sequence of SEQ ID NO: 86. In other embodiments, the caninized antibody against canine IL-31RA or antigen binding fragment thereof comprises a heavy chain comprising the amino acid sequence of SEQ ID NO: 87. In yet other embodiments, the caninized antibody against canine IL-31RA or antigen binding fragment thereof comprises a heavy chain comprising the amino acid sequence of SEQ ID NO: 88. In still other embodiments, the caninized antibody against canine IL-31RA or antigen binding fragment thereof comprises a heavy chain comprising the amino acid sequence of SEQ ID NO: 89.

In yet other embodiments, the caninized antibody comprises a heavy chain comprising the amino acid sequence of SEQ ID NO: 86 and a light chain comprising the amino acid sequence of SEQ ID NO: 90. In still other embodiments, the caninized antibody comprises a heavy chain comprising the amino acid sequence of SEQ ID NO: 86 and a light chain comprising the amino acid sequence of SEQ ID NO: 91. In yet other embodiments, the caninized antibody comprises a heavy chain comprising the amino acid sequence of SEQ ID NO: 86 and a light chain comprising the amino acid sequence of SEQ ID NO: 92.

In still other embodiments, the caninized antibody comprises a heavy chain comprising the amino acid sequence of SEQ ID NO: 87 and a light chain comprising the amino acid sequence of SEQ ID NO: 90. In yet other embodiments, the caninized antibody comprises a heavy chain comprising the amino acid sequence of SEQ ID NO: 87 and a light chain comprising the amino acid sequence of SEQ ID NO: 91. In still other embodiments, the caninized antibody comprises a heavy chain comprising the amino acid sequence of SEQ ID NO: 87 and a light chain comprising the amino acid sequence of SEQ ID NO: 92.

In yet other embodiments, the caninized antibody comprises a heavy chain comprising the amino acid sequence of SEQ ID NO: 88 and a light chain comprising the amino acid sequence of SEQ ID NO: 90. In still other embodiments, the caninized antibody comprises a heavy chain comprising the amino acid sequence of SEQ ID NO: 88 and a light chain comprising the amino acid sequence of SEQ ID NO: 91. In yet other embodiments, the caninized antibody comprises a heavy chain comprising the amino acid sequence of SEQ ID NO: 88 and a light chain comprising the amino acid sequence of SEQ ID NO: 92.

In still other embodiments, the caninized antibody comprises a heavy chain comprising the amino acid sequence of SEQ ID NO: 89 and a light chain comprising the amino acid sequence of SEQ ID NO: 90. In yet other embodiments, the caninized antibody comprises a heavy chain comprising the amino acid sequence of SEQ ID NO: 89 and a light chain comprising the amino acid sequence of SEQ ID NO: 91. In still other embodiments, the caninized antibody comprises a heavy chain comprising the amino acid sequence of SEQ ID NO: 89 and a light chain comprising the amino acid sequence of SEQ ID NO: 92.

In certain embodiments, the caninized antibody against canine IL-31RA or antigen binding fragment thereof comprises a heavy chain comprising the amino acid sequence of SEQ ID NO: 72. In other embodiments, the caninized antibody against canine IL-31RA or antigen binding fragment thereof comprises a heavy chain comprising the amino acid sequence of SEQ ID NO: 73. In still other embodiments, the caninized antibody against canine IL-31RA or antigen binding fragment thereof comprises a heavy chain comprising the amino acid sequence of SEQ ID NO: 74. In yet other embodiments, the caninized antibody against canine IL-31RA or antigen binding fragment thereof comprises a heavy chain comprising the amino acid sequence of SEQ ID NO: 75.

In related embodiments, the caninized antibody against canine TL-3IRA or antigen binding fragment thereof comprises a light chain comprising the amino acid sequence of SEQ ID NO: 76. In other embodiments, the caninized antibody against canine TL-3IRA or antigen binding fragment thereof comprises a light chain comprising the amino acid sequence of SEQ ID NO: 77. In yet other embodiments, the caninized antibody against canine IL-31RA or antigen binding fragment thereof comprises a light chain comprising the amino acid sequence of SEQ ID NO: 78.

In yet other embodiments, the caninized antibody comprises a heavy chain comprising the amino acid sequence of SEQ ID NO: 72 and a light chain comprising the amino acid sequence of SEQ ID NO: 76. In still other embodiments, the caninized antibody comprises a heavy chain comprising the amino acid sequence of SEQ ID NO: 72 and a light chain comprising the amino acid sequence of SEQ ID NO: 77. In yet other embodiments, the caninized antibody comprises a heavy chain comprising the amino acid sequence of SEQ ID NO: 72 and a light chain comprising the amino acid sequence of SEQ ID NO: 78.

In still other embodiments, the caninized antibody comprises a heavy chain comprising the amino acid sequence of SEQ ID NO: 73 and a light chain comprising the amino acid sequence of SEQ ID NO: 76. In yet other embodiments, the caninized antibody comprises a heavy chain comprising the amino acid sequence of SEQ ID NO: 73 and a light chain comprising the amino acid sequence of SEQ ID NO: 77. In still other embodiments, the caninized antibody comprises a heavy chain comprising the amino acid sequence of SEQ ID NO: 73 and a light chain comprising the amino acid sequence of SEQ ID NO: 78.

In yet other embodiments, the caninized antibody comprises a heavy chain comprising the amino acid sequence of SEQ ID NO: 74 and a light chain comprising the amino acid sequence of SEQ ID NO: 76. In still other embodiments, the caninized antibody comprises a heavy chain comprising the amino acid sequence of SEQ ID NO: 74 and a light chain comprising the amino acid sequence of SEQ ID NO: 77. In yet other embodiments, the caninized antibody comprises a heavy chain comprising the amino acid sequence of SEQ ID NO: 74 and a light chain comprising the amino acid sequence of SEQ ID NO: 78.

In still other embodiments, the caninized antibody comprises a heavy chain comprising the amino acid sequence of SEQ ID NO: 75 and a light chain comprising the amino acid sequence of SEQ ID NO: 76. In yet other embodiments, the caninized antibody comprises a heavy chain comprising the amino acid sequence of SEQ ID NO: 75 and a light chain comprising the amino acid sequence of SEQ ID NO: 77. In still other embodiments, the caninized antibody comprises a heavy chain comprising the amino acid sequence of SEQ ID NO: 75 and a light chain comprising the amino acid sequence of SEQ ID NO: 78.

The present invention further provides canine or caninized antibodies or antigen binding fragment thereof that bind to canine interleukin-31 receptor alpha (canine IL-31RA) and block the binding of canine IL-31RA to canine IL-31 and that when bound to canine IL-31RA the antibody binds to an epitope comprised by the amino acid sequence of SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109 or any combination thereof.

In specific embodiments, when bound to canine IL-31RA, the mammalian antibody (e.g., a caninized antibody) binds to an epitope comprised by the amino acid sequence of SEQ ID NO: 104 or SEQ ID NO: 105, or to both SEQ ID NO: 104 and SEQ ID NO: 105; or SEQ ID NO: 98 or SEQ ID NO: 100, or to both SEQ ID NO: 98 and SEQ ID NO: 100; or alternatively to SEQ ID NO: 106 or SEQ ID NO: 108, or to both SEQ ID NO: 106 and SEQ ID NO: 108. The identification of the epitopes can be based on chemical crosslinking and mass spectrometry detection. In related embodiments, when bound to canine IL-31RA, the mammalian antibody binds at least one amino acid residue, preferably one to three amino acid residues, more preferably two to five amino acid residues, and/or more preferably three to eight amino acid residues or more within the amino acid sequence of SEQ ID NO: 104 or within SEQ ID NO: 105, or that are within the amino acid sequences of both SEQ ID NO: 104 and SEQ ID NO: 105; or SEQ ID NO: 98 or SEQ ID NO: 100, or to both SEQ ID NO: 98 and SEQ ID NO: 100; or alternatively, to SEQ ID NO: 106 or SEQ ID NO: 108, or to both SEQ ID NO: 106 and SEQ ID NO: 108.

In more specific embodiments, the mammalian antibody that binds to SEQ ID NO: 104, binds to the arginine residue at position 225 of SEQ ID NO. 2, i.e., R₂₂₅. In other embodiments, the mammalian antibody that binds to SEQ ID NO: 104, binds to the threonine residue at position 236 of SEQ ID NO. 2, i.e., T₂₃₆. In still other embodiments, the mammalian antibody that binds to SEQ ID NO: 105, binds to the arginine residue at position 298 of SEQ ID NO. 2, i.e., R₂₉₈. In yet other embodiments, the mammalian antibody that binds to SEQ ID NO: 105 binds to the lysine residue at position 305 of SEQ ID NO. 2, i.e., K₃₀₅. In still other embodiments, the mammalian antibody that binds to SEQ ID NO: 105 binds to the threonine residue at position 306 of SEQ ID NO. 2, i.e., T₃₀₆. In yet other embodiments, the mammalian antibody binds to at least two amino acid residues from the group consisting of: R₂₂₅ and/or T₂₃₆ and/or R₂₉₈ and/or K₃₀₅ and/or T₃₀₆ of SEQ ID NO: 102. In a specific embodiment of this type, the mammalian antibody binds to R₂₂₅, T₂₃₆, R₂₉₈, K₃₀₅, and T₃₀₆ of SEQ ID NO. 2. The present invention further provides antigen binding fragments of these mammalian antibodies.

In alternative embodiments, the mammalian antibody that binds to SEQ ID NO: 98, binds to the lysine residue at position 102 of SEQ ID NO. 2, i.e., K₁₀₂. In other embodiments, the mammalian antibody that binds to SEQ ID NO: 98, binds to the serine residue at position 110 of SEQ ID NO. 2, i.e., S₁₁₀. In yet other embodiments, the mammalian antibody that binds to SEQ ID NO: 98, binds to the lysine residue at position 112 of SEQ ID NO. 2, i.e., K₁₁₂. In still other embodiments, the mammalian antibody that binds to SEQ ID NO: 98, binds to the lysine residue at position 118 of SEQ ID NO. 2, i.e., K₁₁₈. In yet other embodiments, the mammalian antibody that binds to SEQ ID NO: 98, binds to the arginine residue at position 119 of SEQ ID NO. 2, i.e., R₁₁₉. In still other embodiments, the mammalian antibody that binds to SEQ ID NO: 100, binds to the threonine residue at position 176 of SEQ ID NO. 2, i.e., T₁₇₆. In yet other embodiments, the mammalian antibody that binds to SEQ ID NO: 100 binds to the tyrosine residue at position 178 of SEQ ID NO. 2, i.e., Y₁₇₈. In still other embodiments, the mammalian antibody that binds to SEQ ID NO: 100 binds to the serine residue at position 189 of SEQ ID NO. 2, i.e., S₁₈₉. In yet other embodiments, the mammalian antibody binds to at least two amino acid residues from the group consisting of: K₁₀₂ and/or S₁₁₀ and/or K₁₁₂ and/or K₁₁₈ and/or R₁₁₉ and/or T₁₇₆ and/or Y₁₇₈ and/or S₁₈₉ of SEQ ID NO: 102. In a specific embodiment the mammalian antibody binds to K₁₀₂, S₁₁₀, K₁₁₂, K₁₁₈, R₁₁₉, T₁₇₆, Y₁₇₈, and S₁₈₉ of SEQ ID NO. 2. The present invention further provides antigen binding fragments of these mammalian antibodies.

In still other alternative embodiments, the mammalian antibody that binds to SEQ ID NO: 106, binds to the arginine residue at position 430 of SEQ ID NO. 2, i.e., R₄₃₀. In other embodiments, the mammalian antibody that binds to SEQ ID NO: 106, binds to the lysine residue at position 435 of SEQ ID NO. 2, i.e., K₄₃₅. In yet other embodiments, the mammalian antibody that binds to SEQ ID NO: 106, binds to the threonine residue at position 438 of SEQ ID NO. 2, i.e., T₄₃₈. In still other embodiments, the mammalian antibody that binds to SEQ ID NO: 106, binds to the tyrosine residue at position 441 of SEQ ID NO. 2, i.e., Y₄₄₁. In yet other embodiments, the mammalian antibody that binds to SEQ ID NO: 108, binds to the serine residue at position 472 of SEQ ID NO. 2, i.e., S₄₇₂. In yet other embodiments, the mammalian antibody that binds to SEQ ID NO: 108 binds to the threonine residue at position 479 of SEQ ID NO. 2, i.e., T₄₇₉. In still other embodiments, the mammalian antibody that binds to SEQ ID NO: 108 binds to the threonine residue at position 487 of SEQ ID NO. 2, i.e., T₄₈₇.

In yet other embodiments, the mammalian antibody binds to at least two amino acid residues from the group consisting of: R₄₃₀ and/or K₄₃₅ and/or T₄₃₈ and/or Y₄₄₁ and/or S₄₇₂ and/or T₄₇₉ and/or T₄₈₇ of SEQ ID NO: 102. In a specific embodiment the mammalian antibody binds to R₄₃₀, K₄₃₅, T₄₃₈, Y₄₄₁, S₄₇₂, T₄₇₉, and T₄₈₇ of SEQ ID NO. 2. The present invention further provides antigen binding fragments of these mammalian antibodies.

The present invention also provides nucleic acids, including isolated nucleic acids, that encode any of: the sets of 3 HCDRs or 3 LCDRs; the heavy chain variable regions of the caninized antibodies or antigen binding fragments thereof, the heavy chains of the caninized antibodies or antigen binding fragments thereof, the light chain variable regions of the caninized antibodies or antigen binding fragments thereof, and/or the light chains of the caninized antibodies or antigen binding fragments thereof. The present invention further provides a pair of nucleic acids, wherein one of the pair of nucleic acids comprises a nucleotide sequence that encodes the light chain of a specific caninized antibody of any one of the antibodies of the present invention and the other of the pair of nucleic acids comprises a nucleotide sequence that encodes the heavy chain of that (said) specific caninized antibody. The present invention further provides expression vectors that comprise such pairs of nucleic acids, or alternatively individual nucleic acids of the present invention. In addition, the present invention provides pairs of expression vectors, wherein one of the pair of expression vectors comprises a nucleic acid comprising a nucleotide sequence that encodes the light chain of a specific caninized antibody of any one of the antibodies of the present invention, and the other of the pair of expression vectors comprises a nucleic acid comprising a nucleotide sequence that encodes the heavy chain of that (said) specific caninized antibody.

Accordingly, the present invention provides nucleic acids that encode a set of the three heavy chain complementary determining regions (CDRs), a CDR heavy 1 (HCDR1), a CDR heavy 2 (HCDR2), and a CDR heavy 3 (HCDR3) of a mammalian antibody (including a caninized antibody) of the present invention, nucleic acids that encode a set of the three light chain complementary determining regions (CDRs), a CDR light 1 (LCDR1), a CDR light 2 (LCDR2), and a CDR light 3 (LCDR3) of a mammalian antibody (including of a caninized antibody) or an antigen binding fragment thereof of the present invention, or pairs of such light chains and heavy chain CDRs.

In certain embodiments, the nucleic acid encodes an HCDR1 that comprises the amino acid sequence of SEQ ID NO: 4, an HCDR2 that comprises the amino acid sequence of SEQ ID NO: 14, and an HCDR3 that comprises the amino acid sequence of SEQ ID NO: 24. In related embodiments, the nucleic acid encodes a LCDR1 that comprises the amino acid sequence of SEQ ID NO: 33, a LCDR2 that comprises the amino acid sequence of SEQ ID NO: 37, and the LCDR3 that comprises the amino acid sequence of SEQ ID NO: 46. The present invention further provides as a pair, a nucleic acid encoding this set of the three heavy chain CDRs and a nucleic acid that encodes this set of the three light chain CDRs. The present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

In other embodiments, the nucleic acid encodes an HCDR1 that comprises the amino acid sequence of SEQ ID NO: 5, an HCDR2 that comprises the amino acid sequence of SEQ ID NO: 15, and an HCDR3 that comprises the amino acid sequence of SEQ ID NO: 25. In related embodiments, the nucleic acid encodes a LCDR1 that comprises the amino acid sequence of SEQ ID NO: 32, a LCDR2 that comprises the amino acid sequence of SEQ ID NO: 41, and the LCDR3 that comprises the amino acid sequence of SEQ ID NO: 47. The present invention further provides as a pair, a nucleic acid encoding this set of the three heavy chain CDRs and a nucleic acid that encodes this set of the three light chain CDRs. The present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

In yet other embodiments, the nucleic acid encodes an HCDR1 that comprises the amino acid sequence of SEQ ID NO: 6, an HCDR2 that comprises the amino acid sequence of SEQ ID NO: 16, and an HCDR3 that comprises the amino acid sequence of SEQ ID NO: 26. In related embodiments, the nucleic acid encodes a LCDR1 that comprises the amino acid sequence of SEQ ID NO: 32, a LCDR2 that comprises the amino acid sequence of SEQ ID NO: 42, and the LCDR3 that comprises the amino acid sequence of SEQ ID NO: 48. The present invention further provides as a pair, a nucleic acid encoding this set of the three heavy chain CDRs and a nucleic acid that encodes this set of the three light chain CDRs. The present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

In still other embodiments, the nucleic acid encodes an HCDR1 that comprises the amino acid sequence of SEQ ID NO: 7, an HCDR2 that comprises the amino acid sequence of SEQ ID NO: 17, and an HCDR3 that comprises the amino acid sequence of SEQ ID NO: 27. In related embodiments, the nucleic acid encodes the amino acid sequence of SEQ ID NO: 34, a LCDR2 that comprises the amino acid sequence of SEQ ID NO: 37, and the LCDR3 that comprises the amino acid sequence of SEQ ID NO: 50. The present invention further provides as a pair, a nucleic acid encoding this set of the three heavy chain CDRs and a nucleic acid that encodes this set of the three light chain CDRs. The present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

In yet other embodiments, the nucleic acid encodes an HCDR1 that comprises the amino acid sequence of SEQ ID NO: 8, an HCDR2 that comprises the amino acid sequence of SEQ ID NO: 18, and an HCDR3 that comprises the amino acid sequence of SEQ ID NO: 28. In related embodiments, the nucleic acid encodes a LCDR1 that comprises the amino acid sequence of SEQ ID NO: 35, a LCDR2 that comprises the amino acid sequence of SEQ ID NO: 43, and the LCDR3 that comprises the amino acid sequence of SEQ ID NO: 51. The present invention further provides as a pair, a nucleic acid encoding this set of the three heavy chain CDRs and a nucleic acid that encodes this set of the three light chain CDRs. The present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

In still other embodiments, the nucleic acid encodes an HCDR1 that comprises the amino acid sequence of SEQ ID NO: 9, an HCDR2 that comprises the amino acid sequence of SEQ ID NO: 19, and an HCDR3 that comprises the amino acid sequence of SEQ ID NO: 29. In related embodiments, the nucleic acid encodes a LCDR1 that comprises the amino acid sequence of SEQ ID NO: 36, a LCDR2 that comprises the amino acid sequence of SEQ ID NO: 44, and the LCDR3 that comprises the amino acid sequence of SEQ ID NO: 49. The present invention further provides as a pair, a nucleic acid encoding this set of the three heavy chain CDRs and a nucleic acid that encodes this set of the three light chain CDRs. The present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

The present invention also provides nucleic acids that encode the heavy chain variable region of a mammalian antibody (including a caninized antibody) or an antigen binding fragment thereof of the present invention. The present invention further provides nucleic acids that encode the heavy chain of a mammalian antibody (including a caninized antibody) or an antigen binding fragment thereof of the present invention. The present invention also provides nucleic acids that encode the light chain of a mammalian antibody (including a caninized antibody) or an antigen binding fragment thereof of the present invention. The present invention further provides as a pair, a nucleic acid encoding this set of the three heavy chain CDRs and a nucleic acid that encodes this set of the three light chain CDRs. The present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

In specific embodiments, a nucleic acid of the present invention encodes a heavy chain variable region of a caninized antibody or antigen binding fragment thereof in which the heavy chain variable region comprises the amino acid sequence of SEQ ID NO: 95. In a related embodiment, a nucleic acid encodes the light chain variable region of the caninized antibody or antigen binding fragment thereof in which the light chain variable region comprises the amino acid sequence of SEQ ID NO: 129. The present invention further provides a pair of nucleic acids, wherein one of the pair of nucleic acids comprises a nucleotide sequence that encodes the heavy chain variable region that comprises the amino acid sequence of SEQ ID NO: 95 and the other of the pair of nucleic acids comprises a nucleotide sequence that encodes the light chain variable region that comprises the amino acid sequence of SEQ ID NO: 129. The present invention also provides a kit containing this pair of two nucleic acids.

In other specific embodiments, a nucleic acid of the present invention encodes a heavy chain variable region of a caninized antibody or antigen binding fragment thereof in which the heavy chain variable region comprises the amino acid sequence of SEQ ID NO: 95. In a related embodiment, a nucleic acid encodes the light chain variable region of the caninized antibody or antigen binding fragment thereof in which the light chain variable region comprises the amino acid sequence of SEQ ID NO: 130. The present invention further provides a pair of nucleic acids, wherein one of the pair of nucleic acids comprises a nucleotide sequence that encodes the heavy chain variable region that comprises the amino acid sequence of SEQ ID NO: 95 and the other of the pair of nucleic acids comprises a nucleotide sequence that encodes the light chain variable region that comprises the amino acid sequence of SEQ ID NO: 130. The present invention also provides a kit containing this pair of two nucleic acids.

In still other specific embodiments, a nucleic acid of the present invention encodes a heavy chain variable region of a caninized antibody or antigen binding fragment thereof in which the heavy chain variable region comprises the amino acid sequence of SEQ ID NO: 96. In a related embodiment, a nucleic acid encodes the light chain variable region of the caninized antibody or antigen binding fragment thereof in which the light chain variable region comprises the amino acid sequence of SEQ ID NO: 129. The present invention further provides a pair of nucleic acids, wherein one of the pair of nucleic acids comprises a nucleotide sequence that encodes the heavy chain variable region that comprises the amino acid sequence of SEQ ID NO: 96 and the other of the pair of nucleic acids comprises a nucleotide sequence that encodes the light chain variable region that comprises the amino acid sequence of SEQ ID NO: 129. The present invention also provides a kit containing this pair of two nucleic acids.

In yet other specific embodiments, a nucleic acid of the present invention encodes a heavy chain variable region of a caninized antibody or antigen binding fragment thereof in which the heavy chain variable region comprises the amino acid sequence of SEQ ID NO: 96. In a related embodiment, a nucleic acid encodes the light chain variable region of the caninized antibody or antigen binding fragment thereof in which the light chain variable region comprises the amino acid sequence of SEQ ID NO: 130. The present invention further provides a pair of nucleic acids, wherein one of the pair of nucleic acids comprises a nucleotide sequence that encodes the heavy chain variable region that comprises the amino acid sequence of SEQ ID NO: 96 and the other of the pair of nucleic acids comprises a nucleotide sequence that encodes the light chain variable region that comprises the amino acid sequence of SEQ ID NO: 130. The present invention also provides a kit containing this pair of two nucleic acids.

In specific embodiments, a nucleic acid of the present invention encodes a heavy chain of a caninized antibody or antigen binding fragment thereof in which the heavy chain comprises the amino acid sequence of SEQ ID NO: 88. In a related embodiment, a nucleic acid encodes the light chain of the caninized antibody or antigen binding fragment thereof in which the light chain comprises the amino acid sequence of SEQ ID NO: 91. The present invention further provides a pair of nucleic acids, wherein one of the pair of nucleic acids comprises a nucleotide sequence that encodes the heavy chain that comprises the amino acid sequence of SEQ ID NO: 88 and the other of the pair of nucleic acids comprises a nucleotide sequence that encodes the light chain that comprises the amino acid sequence of SEQ ID NO: 91. The present invention also provides a kit containing this pair of two nucleic acids.

In other specific embodiments, a nucleic acid of the present invention encodes a heavy chain of a caninized antibody or antigen binding fragment thereof in which the heavy chain comprises the amino acid sequence of SEQ ID NO: 88. In a related embodiment, a nucleic acid encodes the light chain of the caninized antibody or antigen binding fragment thereof in which the light chain comprises the amino acid sequence of SEQ ID NO: 92. The present invention further provides a pair of nucleic acids, wherein one of the pair of nucleic acids comprises a nucleotide sequence that encodes the heavy chain that comprises the amino acid sequence of SEQ ID NO: 88 and the other of the pair of nucleic acids comprises a nucleotide sequence that encodes the light chain that comprises the amino acid sequence of SEQ ID NO: 92. The present invention also provides a kit containing this pair of two nucleic acids.

In still other specific embodiments, a nucleic acid of the present invention encodes a heavy chain of a caninized antibody or antigen binding fragment thereof in which the heavy chain comprises the amino acid sequence of SEQ ID NO: 89. In a related embodiment, a nucleic acid encodes the light chain of the caninized antibody or antigen binding fragment thereof in which the light chain comprises the amino acid sequence of SEQ ID NO: 91. The present invention further provides a pair of nucleic acids, wherein one of the pair of nucleic acids comprises a nucleotide sequence that encodes the heavy chain that comprises the amino acid sequence of SEQ ID NO: 89 and the other of the pair of nucleic acids comprises a nucleotide sequence that encodes the light chain that comprises the amino acid sequence of SEQ ID NO: 91. The present invention also provides a kit containing this pair of two nucleic acids.

In yet other specific embodiments, a nucleic acid of the present invention encodes a heavy chain of a caninized antibody or antigen binding fragment thereof in which the heavy chain comprises the amino acid sequence of SEQ ID NO: 89. In a related embodiment, a nucleic acid encodes the light chain of the caninized antibody or antigen binding fragment thereof in which the light chain comprises the amino acid sequence of SEQ ID NO: 92. The present invention further provides a pair of nucleic acids, wherein one of the pair of nucleic acids comprises a nucleotide sequence that encodes the heavy chain that comprises the amino acid sequence of SEQ ID NO: 89 and the other of the pair of nucleic acids comprises a nucleotide sequence that encodes the light chain that comprises the amino acid sequence of SEQ ID NO: 92. The present invention also provides a kit containing this pair of two nucleic acids.

Accordingly, the present invention also provides a kit containing this pair of two nucleic acids. In certain embodiments, a nucleic acid encoding the set of the three heavy chain CDRs encodes the heavy chain of a caninized antibody and the corresponding nucleic acid encoding the set of the three light chain CDRs encodes the light chain of that caninized antibody.

In addition, the present invention provides expression vectors that comprise one or more of the nucleic acids of the present invention that express one or more of the nucleic acids of the present invention, and pairs of those expression vectors. In certain embodiments, one of the pair of expression vectors expresses a heavy chain of a caninized antibody of the present invention and the other expresses a light chain of that caninized antibody. Host cells that comprise the expression vectors of the present invention, including such pairs of such expression vectors are also provided.

The present invention further provides pharmaceutical compositions that comprise the caninized antibodies and antigen binding fragments thereof of the present invention along with a pharmaceutically acceptable carrier and/or diluent. The present invention further provides pharmaceutical compositions that comprise a nucleic acid of the present invention, along with a pharmaceutically acceptable carrier and/or diluent, and/or an expression vector that comprise one or more of the nucleic acids of the present invention, along with a pharmaceutically acceptable carrier and/or diluent.

The present invention also provides methods of treating atopic dermatitis comprising administering one of the aforesaid pharmaceutical compositions to an animal subject that has atopic dermatitis. In particular embodiments, the animal subject is a canine. The present invention also provides methods of aiding in blocking pruritus associated with atopic dermatitis in an animal subject, comprising administering to an animal subject in need thereof of a therapeutically effective amount of a pharmaceutical composition of the present invention. In particular embodiments, the animal subject is a canine.

In addition, the present invention provides methods of producing a caninized antibody or antigen binding fragment thereof that binds canine IL-31RA. In particular embodiments, the method includes culturing a host cell comprising one or more expression vectors that encode and express the light chain of a caninized antibody of the present invention and the heavy chain of that caninized antibody in a culture medium under conditions in which the nucleic acid is expressed, thereby producing a polypeptide comprising the light chain of a caninized antibody of the present invention, and/or the heavy chain of that caninized antibody. The polypeptides are then recovered from the host cell or culture medium. In certain embodiments, the polypeptides comprising the light chain of a caninized antibody of the present invention and the polypeptides comprising the heavy chain of that caninized antibody are combined with each under conditions that are conducive to form a caninized antibody.

These and other aspects of the present invention will be better appreciated by reference to the following Brief Description of the Drawings and the Detailed Description.

### BRIEF DESCRIPTION OF THE DRAWINGS

FIG. 1 shows the binding of IL-31 to IL-31RA. The extracellular domain (ECD) of canine IL-31RA was tested for its ability to bind to canine IL-31. The results indicate that IL-31RA ECD binds in a dose-dependent manner to biotinylated canine IL-31 with an EC50 of 0.55 ng/ml.

FIGS. 2A-2B show the binding of xIL-31RA monoclonal antibodies (mABS) to IL-31RA. The selected mouse mAbs were tested for their reactivity to canine IL-31RA. The results indicate that the selected mouse mAbs bind to canine IL-31RA in a dose-dependent manner. All of the 10 mouse monoclonal antibodies have strong binding reactivity to canine IL-31RA. FIG. 2A depicts mouse mAbs: 51F8 (•), 74H10 (▪), 100H8(▴), 209G5 (▾), 224G3(♦), and Iso control (o). FIG. 2B depicts mouse mAbs: 55B3 (•), 65G9 (▪), 85C10 (▴), 218D9 (▾), 227E7(♦), and Iso control (o).

FIG. 3 shows the blocking of the binding of IL-31 to IL-31RA by monoclonal antibodies (mABS) to IL-31RA. The selected mouse mAbs (anti-canine IL-31RA) were tested for their ability to block the binding of IL-31 with IL-31RA/OSMR by Flow Cytometry. The FACS result indicates that the ten mouse mAbs can block the binding of IL-31 with the IL-31RA/OSMR complex presented on CHO-IL-31RA/OSMR cells. Antibodies 51F8, 74H10, 100H8, 209G5, and 218D9 exhibited superior blocking activity.

FIG. 4 shows the induction of STAT-3 phosphorylation by IL-31. Ba/f3-OI cells expressing the IL-31 receptor complex were tested for IL-31-induced STAT-3 phosphorylation. The results indicate that STAT-3 phosphorylation was induced by IL-31 in the Baf3-OI cells (U) in a dose-dependent manner, implying that: (i) the canine IL-31 receptor complex is successfully expressed on the cell surface; (ii) that the binding of canine IL-31 to the IL-31 receptor can stimulate the endogenous STAT3 phosphorylation; and (iii) then initiate its downstream signaling pathway. Ba/f3 cells (o) were used as the control.

FIGS. 5A-5B show the inhibition of IL-31-mediated STAT-3 phosphorylation in Ba/f3-OI cells by the selected xIL-31RA antibodies. The results indicate that the selected mAbs inhibit IL-31 mediated STAT-3 phosphorylation in a dose-dependent manner in Ba/f3-OI cells. FIG. 5A depicts mouse mAbs 209G5 (▪), 218D9 (♦), 85C10 (▾), and IL-31 protein (♦). FIG. 5B depicts mouse mAbs 100H8 ( [symbol] ), 74H10 (▪), 85C10 (▴), 51F8 (▾), and cIL-31 protein (♦).

FIGS. 6A-6E provides the epitopes on canine IL-31RA for the antibodies 100H8, 51F8, 218D9, 85C10, and 224G3, respectively. FIG. 6A depicts the epitope for 100H8; the epitope comprises the amino acid sequences of SEQ ID NO: 97 (within SEQ ID NO: 119) and SEQ ID NO: 103 (within SEQ ID NO: 120), respectively. FIG. 6B depicts the epitope for 51F8; the epitope comprises comprises the amino acid sequences of SEQ ID NO: 98 (within SEQ ID NO: 121) and SEQ ID NO: 100 (within SEQ ID NO: 122). FIG. 6C depicts the epitope for 218D9; the epitope comprises the amino acid sequences of SEQ ID NO: 104 (within SEQ ID NO: 123) and SEQ ID NO: 105 (within SEQ ID NO: 124), respectively. FIG. 6D depicts the epitope for 85C10; the epitope comprises the amino acid sequences of SEQ ID NO: 106 (within SEQ ID NO: 125) and SEQ ID NO: 108 (within SEQ ID NO: 126), respectively. FIG. 6E depicts the epitope for 224G3; the epitope comprises the amino acid sequence of SEQ ID NO: 109 (also within SEQ ID NO: 126). The position of binding residues of the amino acid sequence of SEQ ID NO: 2 for the respective epitopes on the cIL-31R ECD antigen are also denoted.

FIGS. 7A-7E provides plots for the binding activity of the identified murine-canine chimeric or caninized antibodies to canine IL-31RA. The results indicate that the caninized antibodies have similar binding affinity as their corresponding parental antibodies (as represented by the murine-canine chimeric antibodies). FIG. 7A depicts the binding plots for monoclonal 51F8 antibodies: Chimeric 51F8 (•) c51F8VH3VL6 (▪), c51F8VH3VL7 (▴), c51F8VH4VL6 (▾), and c51F8VH4VL7 (♦); and the iso control (o). FIG. 7B depicts the binding plots for monoclonal 100H8 antibodies: Chimeric 100H8 (•), c100H8VH5VL4 (▪), and c100H8VH7VL4 (▴). FIG. 7C depicts the binding plots for monoclonal 85C10 antibodies: Chimeric 85C10 (•), c85C10VH3VL2 (▪), and c85C10VH1VL2(▴). FIG. 7D depicts the binding plots for monoclonal 218D9 antibodies: Chimeric 218D9 (•), c218D9VH3VL2 (▪), c218D9VH3VL3 (▴), c218D9VH4VL2 (▾), and c218D9VH4VL3 (♦); and the iso control (o). FIG. 7E depicts the binding plots for monoclonal 224G3 antibodies: m224G3 Chim (•), c224G3VH2VL2 (▪), and c224G3VH2VL3(▴).

The term “Chimeric” before the antibody number signifies that the antibody is a murine-canine chimeric antibody, e.g., Chimeric 218D9 or Chimeric 51F8. In addition, an “m” before the antibody number followed by a “Chim” signifies that the antibody is a murine-canine chimeric antibody, e.g., m224G3 Chim. The lower case “c” before the antibody number signifies that it is a caninized antibody, e.g., c218D9VH4VL2.

FIG. 8 shows the blocking of the binding of IL-31 to IL-31RA by the inhibition of the IL-31-mediated STAT-3 phosphorylation in Ba/f3-OI cells. The results indicate that the caninized 218D9 antibodies can inhibit IL-31 mediated STAT-3 phosphorylation in a dose-dependent manner in Ba/f3-OI cells, and that the constructs c218D9VH3VL3 and c218D9VH4VL3 have the same inhibitory activity as the parental mouse-canine chimeric 218D9 antibody: Chimeric 218D9 (e), c218D9VH3VL2 (U), c218D9VH3VL3 (A), c218D9VH4VL2 (V), and c218D9VH4VL3 (+); and the IL-31 only control (o).

### DETAILED DESCRIPTION OF THE INVENTION

In response to need for better therapies for atopic dermatitis, the present invention provides formulations and methodology that can achieve a significant effect on the skin inflammation associated with atopic dermatitis.

### Abbreviations

Throughout the detailed description and examples of the invention the following abbreviations will be used:
ADCC Antibody-dependent cellular cytotoxicity
CDC Complement-dependent cyotoxicity
CDR Complementarity determining region in the immunoglobulin variable regions, defined using the Kabat numbering system
EC50 concentration resulting in 50% efficacy or binding
ELISA Enzyme-linked immunosorbant assay
FR Antibody framework region: the immunoglobulin variable regions excluding the CDR regions.
IC50 concentration resulting in 50% inhibition
IgG Immunoglobulin G
Kabat An immunoglobulin alignment and numbering system pioneered by Elvin A. Kabat [Sequences of Proteins of Immunological Interest, 5th Ed. Public Health Service, National Institutes of Health, Bethesda, Md. (1991)]
mAb Monoclonal antibody (also Mab or MAb)
V region The segment of IgG chains which is variable in sequence between different antibodies.
VH Immunoglobulin heavy chain variable region
VL Immunoglobulin light chain variable region

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Administration” and “treatment”, as it applies to an animal, e.g., a canine subject, cell, tissue, organ, or biological fluid, refers to contact of an exogenous pharmaceutical, therapeutic, diagnostic agent, or composition to the animal e.g., a canine subject, cell, tissue, organ, or biological fluid. Treatment of a cell encompasses contact of a reagent to the cell, as well as contact of a reagent to a fluid, where the fluid is in contact with the cell.

“Administration” and “treatment” also mean in vitro and ex vivo treatments, e.g., of a cell, by a reagent, diagnostic, binding compound, or by another cell. The term “subject” includes any organism, preferably an animal, more preferably a mammal (e.g., canine or feline) and most preferably a canine.

“Treat” or “treating” means to administer a therapeutic agent, such as a composition containing any of the antibodies of the present invention, internally or externally to e.g., a canine subject or patient having one or more symptoms, or being suspected of having a condition, for which the agent has therapeutic activity. Typically, the agent is administered in an amount effective to alleviate and/or ameliorate one or more disease/condition symptoms in the treated subject or population, whether by inducing the regression of or inhibiting the progression of such symptom(s) by any clinically measurable degree. The amount of a therapeutic agent that is effective to alleviate any particular disease/condition symptom (also referred to as the “therapeutically effective amount”) may vary according to factors such as the disease/condition state, age, and weight of the patient (e.g., canine), and the ability of the pharmaceutical composition to elicit a desired response in the subject. Whether a disease/condition symptom has been alleviated or ameliorated can be assessed by any clinical measurement typically used by veterinarians or other skilled healthcare providers to assess the severity or progression status of that symptom. While an embodiment of the present invention (e.g., a treatment method or article of manufacture) may not be effective in alleviating the target disease/condition symptom(s) in every subject, it should alleviate the target disease/condition symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary (e.g., canine), or research subject, refers to therapeutic treatment, as well as research and diagnostic applications. “Treatment” as it applies to a human, veterinary (e.g., canine), or research subject, or cell, tissue, or organ, encompasses contact of the antibodies of the present invention to e.g., a canine or other animal subject, a cell, tissue, physiological compartment, or physiological fluid.

As used herein, the term “canine” includes all domestic dogs, Canis lupus familiaris or Canis familiaris, unless otherwise indicated.

As used herein, the term “feline” refers to any member of the Felidae family. Members of this family include wild, zoo, and domestic members, including domestic cats, pure-bred and/or mongrel companion cats, show cats, laboratory cats, cloned cats, and wild or feral cats.

As used herein the term “canine frame” refers to the amino acid sequence of the heavy chain and light chain of a canine antibody other than the hypervariable region residues defined herein as CDR residues. With regard to a caninized antibody, in the majority of embodiments the amino acid sequences of the native canine CDRs are replaced with the corresponding foreign CDRs (e.g., those from a mouse or rat antibody) in both chains. Optionally the heavy and/or light chains of the canine antibody may contain some foreign non-CDR residues, e.g., so as to preserve the conformation of the foreign CDRs within the caninized antibody, and/or to modify the Fc function, as exemplified below and/or disclosed in U.S. Pat. No. 10,106,607 B2, hereby incorporated by reference herein in its entirety.

The “Fragment crystallizable region” abbreviated as “Fc” or as used interchangeably “Fc region” corresponds to the CH3-CH2 portion of an antibody that interacts with cell surface receptors called Fc receptors. The canine fragment crystallizable region (cFc) of each of the four canine IgGs were first described by Tang et al. [Vet. Immunol. Immunopathol. 80: 259-270 (2001); see also, Bergeron et al., Vet. Immunol. Immunopathol. 157: 31-41 (2014) and U.S. Pat. No. 10,106,607 B2].

As used herein the canine Fc (cFc) “IgG-Bm” is canine IgG-B Fc comprising two (2) amino acid residue substitutions, D31A and N63A, as in the amino acid sequence of SEQ ID NO: 111 of IgG-B (see below) and without the c-terminal lysine (“K”). Both the aspartic acid residue (D) at position 31 of SEQ ID NO: 110 and the asparagine residue (N) at position 63 of SEQ ID NO: 110, are substituted by an alanine residue (A) in IgG-Bm. These two amino acid residue substitutions serve to significantly diminish the antibody-dependent cytotoxicity (ADCC) and complement-dependent cytotoxicity (CDC) of the naturally occurring canine IgG-B [see, U.S. Pat. No. 10,106,607 B2, the contents of which are hereby incorporated by reference in their entirety]. The amino acid sequence of IgG-B, SEQ ID NO: 110 is:

1 50 [symbol]
PKPKDTLLIA RTPEVTCVVV DLDPEDPEVQ ISWFVDGKQM
51 100
QTAKTQPREE QFNGTYRVVS VLPIGHQDWL KGKQFTCKVN NKALPSPIER
101 150
TISKARG [symbol] QPSVYVLPPS REELSKNTVS LTCLIKDFFP PDIDVEWQSN
151 200
GQQEPESKYR TTPPQLDEDG SYFLYSKLSV DKSRWQRGDT FICAVMHEAL
201 215
HNHYTQESLS HSPGK

The amino acid sequence of IgG-Bm, SEQ ID NO: 111, is provided below.

LGGPSVFIFPPKPKDTLLIARTPEVTCVVVALDPEDPEVQISWFVDGKQ
MQTAKTQPREEQFAGTYRVVSVLPIGHQDWLKGKQFTCKVNNKALPSPI
ERTISKARGQAHQPSVYVLPPSREELSKNTVSLTCLIKDFFPPDIDVEW
QSNGQQEPESKYRTTPPQLDEDGSYFLYSKLSVDKSRWQRGDTFICAVM
HEALHNHYTQESLSHSPG

As used herein, a “substitution of an amino acid residue” with another amino acid residue in an amino acid sequence of an antibody for example, is equivalent to “replacing an amino acid residue” with another amino acid residue and denotes that a particular amino acid residue at a specific position in the amino acid sequence has been replaced by (or substituted for) by a different amino acid residue. Such substitutions can be particularly designed i.e., purposefully replacing an alanine with a serine at a specific position in the amino acid sequence by e.g., recombinant DNA technology. Alternatively, a particular amino acid residue or string of amino acid residues of an antibody can be replaced by one or more amino acid residues through more natural selection processes e.g., based on the ability of the antibody produced by a cell to bind to a given region on that antigen, e.g., one containing an epitope or a portion thereof, and/or for the antibody to comprise a particular CDR that retains the same canonical structure as the CDR it is replacing. Such substitutions/replacements can lead to “variant” CDRs and/or variant antibodies.

As used herein, the term “antibody” refers to any form of antibody that exhibits the desired biological activity. An antibody can be a monomer, dimer, or larger multimer. Thus, it is used in the broadest sense and specifically covers, but is not limited to, monoclonal antibodies (including full length monoclonal antibodies), polyclonal antibodies, multi-specific antibodies (e.g., bispecific antibodies), caninized antibodies, fully canine antibodies, chimeric antibodies and camelized single domain antibodies. “Parental antibodies” are antibodies obtained by exposure of an immune system to an antigen prior to modification of the antibodies for an intended use, such as caninization of an antibody for use as a canine therapeutic antibody.

As used herein, antibodies of the present invention that “block” or is “blocking” or is “blocking the binding” of e.g., a canine receptor to its binding partner (ligand), is an antibody that blocks (partially or fully) the binding of the canine receptor to its canine ligand and vice versa, as determined in standard binding assays (e.g., BIACore®, ELISA, or flow cytometry).

Typically, an antibody or antigen binding fragment of the invention retains at least 10% of its canine antigen binding activity (when compared to the parental antibody) when that activity is expressed on a molar basis. Preferably, an antibody or antigen binding fragment of the invention retains at least 20%, 50%, 70%, 80%, 90%, 95% or 100% or more of the canine antigen binding affinity as the parental antibody. It is also intended that an antibody or antigen binding fragment of the invention can include conservative or non-conservative amino acid substitutions (referred to as “conservative variants” or “function conserved variants” of the antibody) that do not substantially alter its biologic activity.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

As used herein, an antibody is said to bind specifically to a polypeptide comprising a given antigen sequence (in this case a portion of the amino acid sequence of canine IL-31RA) if it binds to polypeptides comprising the portion of the amino acid sequence of canine IL-31RA, but does not bind to other canine proteins lacking that portion of the sequence of canine IL-31RA. For example, an antibody that specifically binds to a polypeptide comprising canine IL-31RA, may bind to a FLAG®-tagged form of canine IL-31RA, but will not bind to other FLAG®-tagged canine proteins.

As used herein, unless otherwise indicated, “antibody fragment” or “antigen binding fragment” refers to antigen binding fragments of antibodies, i.e. antibody fragments that retain the ability to bind specifically to the antigen (e.g., canine IL-31RA) bound by the full-length antibody, e.g. fragments that retain one or more CDR regions. Examples of antigen binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody, or binding compound derived from the antigen-binding site of an antibody, binds to its canine antigen, or a variant or mutein thereof, “with specificity” when it has an affinity for that canine antigen or a variant or mutein thereof which is at least ten-times greater, more preferably at least 20-times greater, and even more preferably at least 100-times greater than its affinity for any other canine antigen tested.

As used herein, a “chimeric antibody” is an antibody having the variable domain from a first antibody and the constant domain from a second antibody, where the first and second antibodies are from different species. [U.S. Pat. No. 4,816,567; and Morrison et al., Proc. Natl. Acad. Sci. USA 81: 6851-6855 (1984)]. Typically the variable domains are obtained from an antibody from an experimental animal (the “parental antibody”), such as a rodent, and the constant domain sequences are obtained from the animal subject antibodies, e.g., human or canine so that the resulting chimeric antibody will be less likely to elicit an adverse immune response in a human or canine subject respectively, than the parental (e.g., rodent) antibody.

As used herein, the term “caninized antibody” refers to forms of antibodies that contain sequences from both canine and non-canine (e.g., mouse) antibodies. In general, the caninized antibody will comprise substantially all of at least one or more typically, two variable domains in which all or substantially all of the hypervariable loops correspond to those of a non-canine immunoglobulin (e.g., comprising 6 CDRs as exemplified below), and all or substantially all of the framework (FR) regions (and typically all or substantially all of the remaining frame) are those of a canine immunoglobulin sequence. As exemplified herein, a caninized antibody comprises both the three heavy chain CDRs and the three light chain CDRS from a murine (mouse) anti-canine antigen antibody together with a canine frame or a modified canine frame. A modified canine frame comprises one or more amino acids changes as exemplified herein that further optimize the effectiveness of the caninized antibody, e.g., to increase its binding to its canine antigen and/or its ability to block the binding of that canine antigen to the canine antigen's natural binding partner.

The variable regions of each light/heavy chain pair form the antibody binding site. Thus, in general, an intact antibody has two binding sites. Except in bifunctional or bispecific antibodies, the two binding sites are, in general, the same. Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat, Adv. Prot. Chem. 32:1-75 (1978); Kabat, et al., J. Biol. Chem. 252:6609-6616 (1977); Chothia, et al., J. Mol. Biol. 196:901-917 (1987) or Chothia, et al., Nature 342:878-883 (1989)].

As used herein, the term “hypervariable region” refers to the amino acid residues of an antibody that are responsible for antigen-binding. The hypervariable region comprises amino acid residues from a “complementarity determining region” or “CDR” (i.e. LCDR1, LCDR2 and LCDR3 in the light chain variable domain and HCDR1, HCDR2 and HCDR3 in the heavy chain variable domain). [See Kabat et al. Sequences of Proteins of Immunological Interest, 5th Ed. Public Health Service, National Institutes of Health, Bethesda, Md. (1991), defining the CDR regions of an antibody by sequence; see also Chothia and Lesk, J. Mol. Biol. 196: 901-917 (1987) defining the CDR regions of an antibody by structure]. As used herein, the term “framework” or “FR” residues refers to those variable domain residues other than the hypervariable region residues defined herein as CDR residues.

There are four known IgG heavy chain subtypes of dog IgG and they are referred to as IgG-A, IgG-B, IgG-C, and IgG-D. The two known light chain subtypes are referred to as lambda and kappa. In specific embodiments of the invention, besides binding canine IL-31RA, a canine or caninized antibody against its antigen of the present invention optimally has two attributes:
1) Lack of effector functions such as antibody-dependent cytotoxicity (ADCC) and complement-dependent cytotoxicity (CDC), and
2) be readily purified on a large scale using industry standard technologies such as that based on protein A chromatography.

None of the naturally occurring canine IgG isotypes satisfy both criteria. For example, IgG-B can be purified using protein A, but has high level of ADCC activity. On the other hand, IgG-A binds weakly to protein A, but also displays ADCC activity. Moreover, neither IgG-C nor IgG-D can be purified on protein A columns, although IgG-D displays no ADCC activity. (IgG-C has considerable ADCC activity). One way the present invention addresses these issues in certain embodiments is by providing modified canine IgG-B antibodies of the present invention specific to an antigen of the present invention that lack the effector functions such as ADCC and can be easily purified using industry standard protein A chromatography.

As used herein an “antipruritic agent” is a compound, macromolecule, and/or formulation that tends to inhibit, relieve, and/or prevent itching. Antipruritic agents are colloquially referred to as anti-itch drugs.

As used herein an “antipruritic antibody” is an antibody that can act as an antipruritic agent in an animal, including a mammal such as a human, a canine, and/or a feline, particularly with respect to atopic dermatitis. In particular embodiments, the antipruritic antibody binds to specific proteins in the IL-31 signaling pathway, such as IL-31 or its receptor IL-31RA. The binding of the antipruritic antibody to its corresponding antigen (e.g., IL-31 or IL-31RA) inhibits the binding of e.g., IL-31 with IL-31RA, and interferes with and/or prevents the successful signaling of this pathway, and thereby inhibits, relieves, and/or prevents the itching that is otherwise caused by the IL-31 signaling pathway.

“Homology”, as used herein, refers to sequence similarity between two polynucleotide sequences or between two polypeptide sequences when they are optimally aligned. When a position in both of the two compared sequences is occupied by the same base or amino acid residue, e.g., if a position in each of two DNA molecules is occupied by adenine, then the molecules are homologous at that position. The percent of homology is the number of homologous positions shared by the two sequences divided by the total number of positions compared ×100. For example, if 6 of 10 of the positions in two sequences are matched or homologous when the sequences are optimally aligned then the two sequences are 60% homologous. Generally, the comparison is made when two sequences are aligned to give maximum percent homology.

Sequence identity refers to the degree to which the amino acids of two polypeptides are the same at equivalent positions when the two sequences are optimally aligned. As used herein one amino acid sequence is 100% “identical” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In particular embodiments, selected deletions or insertions that could otherwise alter the correspondence between the two amino acid sequences are taken into account.

Sequence similarity includes identical residues and nonidentical, biochemically related amino acids. Biochemically related amino acids that share similar properties and may be interchangeable.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity [see, e.g., Watson et al., Molecular Biology of the Gene, The Benjamin/Cummings Pub. Co., p. 224 (4th Ed.; 1987)]. In addition, substitutions of structurally or functionally similar amino acids are less likely to disrupt biological activity. Exemplary conservative substitutions are set forth in Table A directly below.

TABLE A
EXEMPLARY CONSERVATIVE AMINO
ACID SUBSTITUTIONS

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala; Gly |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the antibodies of the invention are also contemplated by the present invention. “Function-conservative variants,” as used herein, refers to antibodies or fragments in which one or more amino acid residues have been changed without altering a desired property, such an antigen affinity and/or specificity. Such variants include, but are not limited to, replacement of an amino acid with one having similar properties, such as the conservative amino acid substitutions of Table A above.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The present invention provides isolated caninized antibodies of the present invention, methods of use of the antibodies in the treatment of a condition e.g., the treatment of atopic dermatitis in canines. In canine, there are four IgG heavy chains referred to as A, B, C, and D. These heavy chains represent four different subclasses of dog IgG, which are referred to as IgG-A (or IgGA), IgG-B (or IgGB), IgG-C(or IgGC) and IgG-D (or IgGD). Each of the two heavy chains consists of one variable domain (VH) and three constant domains referred to as CH-1, CH-2, and CH-3. The CH-1 domain is connected to the CH-2 domain via an amino acid sequence referred to as the “hinge” or alternatively as the “hinge region”.

The nucleic acid and amino acid sequences of these four heavy chains were first identified by Tang et al. [Vet. Immunol. Immunopathol. 80: 259-270 (2001)]. The amino acid and nucleic sequences for these heavy chains are also available from the GenBank data bases. For example, the amino acid sequence of IgGA heavy chain has accession number AAL35301.1, IgGB has accession number AAL35302.1, IgGC has accession number AAL35303.1, and IgGD has accession number (AAL35304.1). Canine antibodies also contain two types of light chains, kappa and lambda. The DNA and amino acid sequence of these light chains can be obtained from GenBank Databases. For example, the kappa light chain amino acid sequence has accession number ABY 57289.1 and the lambda light chain has accession number ABY 55569.1.

The known amino acid sequences of the four unmodified canine IgGs are:

cIgG-A
[SEQ ID NO: 116]
LGGPSVLIFPPKPKDILRITRTPEVTCVVLDLGREDPEVQISWFVDGK
EVHTAKTQSREQQFNGTYRVVSVLPIEHQDWLTGKEFKCRVNHIDLPS
PIERTISKARGRAHKPSVYVLPPSPKELSSSDTVSITCLIKDFYPPDI
DVEWQSNGQQEPERKHRMTPPQLDEDGSYFLYSKLSVDKSRWQQGDPF
TCAVMHETLQNHYTDLSLSHSPGK
cIgG-B
[SEQ ID NO: 110]
LGGPSVFIFPPKPKDTLLIARTPEVTCVVVDLDPEDPEVQISWFVDGK
QMQTAKTQPREEQFNGTYRVVSVLPIGHQDWLKGKQFTCKVNNKALPS
PIERTISKARGQAHQPSVYVLPPSREELSKNTVSLTCLIKDFFPPDID
VEWQSNGQQEPESKYRTTPPQLDEDGSYFLYSKLSVDKSRWQRGDTFI
CAVMHEALHNHYTQESLSHSPGK
cIgG-C
[SEQ ID NO: 117]
LGGPSVFIFPPKPKDILVTARTPTVTCVVVDLDPENPEVQISWFVDSK
QVQTANTQPREEQSNGTYRVVSVLPIGHQDWLSGKQFKCKVNNKALPS
PIEEIISKTPGQAHQPNVYVLPPSRDEMSKNTVTLTCLVKDFFPPEID
VEWQSNGQQEPESKYRMTPPQLDEDGSYFLYSKLSVDKSRWQRGDTFI
CAVMHEALHNHYTQISLSHSPGK
cIgG-D
[SEQ ID NO: 118]
LGGPSVFIFPPKPKDILRITRTPEITCVVLDLGREDPEVQISWFVDGK
EVHTAKTQPREQQFNSTYRVVSVLPIEHQDWLTGKEFKCRVNHIGLPS
PIERTISKARGQAHQPSVYVLPPSPKELSSSDTVTLTCLIKDFFPPEI
DVEWQSNGQPEPESKYHTTAPQLDEDGSYFLYSKLSVDKSRWQQGDTF
TCAVMHEALQNHYTDLSLSHSPGK

An amino acid sequence of the kappa canine light chain constant region is:

[SEQ ID NO: 127]
RNDAQPAVYLFQPSPDQLHTGSASVVCLLNSFYPKDINVKWKVDGVIQ
DTGIQESVTEQDKDSTYSLSSTLTMSSTEYLSHELYSCEITHKSLPST
LIKSFQRSECQRVD

In the present invention, the amino acid sequence for each of the four canine IgG Fc fragments is based on the identified boundary of CH1 and CH2 domains as determined by Tang et al, supra. Caninized mouse anti-canine antibodies that bind canine IL-31RA include, but are not limited to: antibodies of the present invention that comprise canine IgG-A, IgG-B, IgG-C, and IgG-D heavy chains and/or canine kappa or lambda light chains together with mouse anti-canine IL-31RA CDRs. Accordingly, the present invention provides caninized mouse anti-canine antibodies of the present invention, including isolated caninized mouse anti-canine antibodies, that bind to canine IL-31RA and that preferably also block the binding of that canine IL-31RA to canine IL-31.

Accordingly, the present invention further provides caninized mouse antibodies and methods of use of the antibodies of the present invention in the treatment of a condition e.g., the treatment of atopic dermatitis in canines.

The present invention further provides full length caninized heavy chains that can be matched with corresponding light chains to make a caninized antibody. Accordingly, the present invention further provides caninized mouse anti-canine antigen antibodies (including isolated caninized mouse anti-canine antibodies) of the present invention and methods of use of the antibodies of the present invention in the treatment of a condition e.g., the treatment of atopic dermatitis in canines.

The present invention also provides antibodies of the present invention that comprise a canine fragment crystallizable region (cFc region) in which the cFc has been genetically modified to augment, decrease, or eliminate one or more effector functions. In one aspect of the present invention, the genetically modified cFc decreases or eliminates one or more effector functions. In another aspect of the invention the genetically modified cFc augments one or more effector function. In certain embodiments, the genetically modified cFc region is a genetically modified canine IgGB Fc region. In another such embodiment, the genetically modified cFc region is a genetically modified canine IgGC Fc region. In a particular embodiment the effector function is antibody-dependent cytotoxicity (ADCC) that is augmented, decreased, or eliminated.

In another embodiment the effector function is complement-dependent cytotoxicity (CDC) that is augmented, decreased, or eliminated. In yet another embodiment, the cFc region has been genetically modified to augment, decrease, or eliminate both the ADCC and the CDC.

In order to generate variants of canine IgG that lack effector functions, a number of mutant canine IgGB heavy chains were generated. These variants may include one or more of the following single or combined substitutions in the Fc portion of the heavy chain amino acid sequence: P4A, D31A, N63A, G64P, T65A, A93G, and P95A. Variant heavy chains (i.e., containing such amino acid substitutions) are cloned into expression plasmids and are transfected into HEK 293 cells along with a plasmid containing the gene encoding a light chain. Intact antibodies are expressed and purified from HEK 293 cells and then can be evaluated for binding to Fc_{γ}RI and C1q to assess their potential for mediation of immune effector functions. [See, U.S. Pat. No. 10,106,607 B2, the contents of which are hereby incorporated by reference in its entirety.]

The present invention also provides modified canine IgG-Ds which in place of its natural IgG-D hinge region they comprise a hinge region from:

IgG-A:
SEQ ID NO: 112
FNECRCTDTPPCPVPEP
IgG-B:
SEQ ID NO: 113
PKRENGRVPRPPDCPKCPAPEM;
or
IgG-C:
SEQ ID NO: 114
AKECECKCNCNNCPCPGCGL.

Alternatively, the IgG-D hinge region can be genetically modified by replacing a serine residue with a proline residue, i.e., PKESTCKCIPPCPVPES, SEQ ID NO: 115 (with the proline residue (P) underlined and in bold substituting for the naturally occurring serine residue). Such modifications can lead to a canine IgG-D lacking fab arm exchange. The modified canine IgG-Ds can be constructed using standard methods of recombinant DNA technology [e.g., Maniatis et al., Molecular Cloning, A Laboratory Manual (1982)]. In order to construct these variants, the nucleic acids encoding the amino acid sequence of canine IgG-D can be modified so that it encodes the modified IgG-Ds. The modified nucleic acid sequences are then cloned into expression plasmids for protein expression.

The six complementary determining regions (CDRs) of a caninized mouse anti-canine antibody, as described herein can comprise a canine antibody kappa (k) or lambda (l) light chain comprising a mouse light chain LCDR1, LCDR2, and LCDR3 and a canine antibody heavy chain IgG comprising a mouse heavy chain HCDR1, HCDR2, and HCDR3.

### Nucleic Acids

The present invention further comprises the nucleic acids encoding the antibodies of the present invention (see e.g., Examples below).

Also included in the present invention are nucleic acids that encode immunoglobulin polypeptides comprising amino acid sequences that are at least about 70% identical, preferably at least about 80% identical, more preferably at least about 90% identical and most preferably at least about 95% identical (e.g., 95%, 96%, 97%, 98%, 99%, 100%) to the amino acid sequences of the caninized antibodies, with the exception of the CDRs which do not change, provided herein when the comparison is performed by a BLAST algorithm wherein the parameters of the algorithm are selected to give the largest match between the respective sequences over the entire length of the respective reference sequences. The present invention further provides nucleic acids that encode immunoglobulin polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference amino acid sequences when the comparison is performed with a BLAST algorithm, wherein the parameters of the algorithm are selected to give the largest match between the respective sequences over the entire length of the respective reference sequences, are also included in the present invention.

As used herein, nucleotide and amino acid sequence percent identity can be determined using C, MacVector (MacVector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

The following references relate to BLAST algorithms often used for sequence analysis: BLAST ALGORITHMS: Altschul, S. F., et al., J. Mol. Biol. 215:403-410 (1990); Gish, W., et al., Nature Genet. 3:266-272 (1993); Madden, T. L., et al., Meth. Enzymol. 266:131-141(1996); Altschul, S. F., et al., Nucleic Acids Res. 25:3389-3402 (1997); Zhang, J., et al., Genome Res. 7:649-656 (1997); Wootton, J. C., et al., Comput. Chem. 17:149-163 (1993); Hancock, J. M. et al., Comput. Appl. Biosci. 10:67-70 (1994); ALIGNMENT SCORING SYSTEMS: Dayhoff, M. O., et al., “A model of evolutionary change in proteins.” 5 in Atlas of Protein Sequence and Structure, vol. 5, suppl. 3. M. O. Dayhoff (ed.), pp. 345-352, (1978); Natl. Biomed. Res. Found., Washington, DC; Schwartz, R. M., et al., “Matrices for detecting distant relationships.” in Atlas of Protein Sequence and Structure, vol. 5, suppl. 3.” (1978), M. O. Dayhoff (ed.), pp. 353-358 (1978), Natl. Biomed. Res. Found., Washington, DC; Altschul, S. F., J. Mol. Biol. 219:555-565 (1991); States, D. J., et al., Methods 3:66-70(1991); Henikoff, S., et al., Proc. Natl. Acad. Sci. USA 89:10915-10919 (1992); Altschul, S. F., et al., J. Mol. Evol. 36:290-300 (1993); ALIGNMENT STATISTICS: Karlin, S., et al., Proc. Natl. Acad. Sci. USA 87:2264-2268 (1990); Karlin, S., et al., Proc. Natl. Acad. Sci. USA 90:5873-5877 (1993); Dembo, A., et al., Ann. Prob. 22:2022-2039 (1994); and Altschul, S. F. “Evaluating the statistical significance of multiple distinct local alignments.” in Theoretical and Computational Methods in Genome Research (S. Suhai, ed.), pp. 1-14, Plenum, New York (1997).

### Antibody Protein Engineering

By way of example, and not limitation, the canine heavy chain constant region can be from IgGA, IgG-B, IgGC, IgGD, or a modified cFc, such as the IgG-Bm used herein [see, U.S. Pat. No. 10,106,607 B2, hereby incorporated by reference in its entirety] and the canine light chain constant region can be from kappa or lambda.

The antibodies can be engineered to include modifications to the canine framework and/or the canine frame residues within the variable domains of a parental (i.e., mouse) monoclonal antibody, e.g. to improve the properties of the antibody.

The construction of caninized anti-canine IL-31 receptor alpha monoclonal antibodies can be performed by determining a DNA sequence that encodes the heavy and light chains of canine IgG were determined. The DNA and protein sequence of the canine heavy and light chains are known in the art and can be obtained by searching of the NCBI gene and protein databases. As indicated above, for canine antibodies there are four known IgG subtypes: IgG-A, IgG-B, IgG-C, and IgG-D, and two types of light chains, i.e., kappa and lambda.

A caninized mouse anti-canine TL-3IRA antibody can be produced recombinantly by methods that are known in the field. Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

In certain embodiments, the antibody or antigen binding fragment comprises a heavy chain constant region, e.g., a canine constant region, such as IgG-A, IgG-B, IgG-C and IgG-D canine heavy chain constant region or a variant thereof. In certain embodiments, the antibody or antigen binding fragment comprises a light chain constant region, e.g., a canine light chain constant region, such as lambda or kappa canine light chain region or variant thereof. By way of example, and not limitation, the canine heavy chain constant region can be from IgG-B and the canine light chain constant region can be from kappa.

### Epitope Mapping

The interaction of antibodies with their cognate protein antigens is mediated through the binding of specific amino acids of the antibodies (paratopes) with specific amino acids (epitopes) of target antigens. An epitope is an antigenic determinant that causes a specific reaction by an immunoglobulin. An epitope consists of a group of amino acids on the surface of the antigen. A protein of interest may contain several epitopes that are recognized by different antibodies. The epitopes recognized by antibodies are classified as linear or conformational epitopes. Linear epitopes are formed by a stretch of a continuous sequence of amino acids in a protein, while conformational epitopes are composed of amino acids that are discontinuous (e.g., far apart) in the primary amino acid sequence, but are brought together upon three-dimensional protein folding.

Epitope mapping refers to the process of identifying the amino acid sequences (i.e., epitopes) that are recognized by antibodies on their target antigens. Identification of epitopes recognized by monoclonal antibodies (mAbs) on target antigens has important applications. For example, it can aid in the development of new therapeutics, diagnostics, and vaccines. Epitope mapping can also aid in the selection of optimized therapeutic mAbs and help elucidate their mechanisms of action. Epitope information on IL-31 receptor alpha can also elucidate unique epitopes and define the protective or pathogenic effects of vaccines. Epitope identification also can lead to development of subunit vaccines based on chemical or genetic coupling of the identified peptide epitope to a carrier protein or other immunostimulating agents.

Epitope mapping can be carried out using polyclonal or monoclonal antibodies and several methods are employed for epitope identification depending on the suspected nature of the epitope (i.e., linear versus conformational). Mapping linear epitopes is more straightforward and relatively, easier to perform. For this purpose, commercial services for linear epitope mapping often employ peptide scanning. In this case, an overlapping set of short peptide sequences of the target protein are chemically synthesized and tested for their ability to bind antibodies of interest. The strategy is rapid, high-throughput, and relatively inexpensive to perform. On the other hand, mapping of a discontinuous epitope is more technically challenging and requires more specialized techniques such as x-ray co-crystallography of a monoclonal antibody together with its target protein, Hydrogen-Deuterium (H/D) exchange, Mass Spectrometry coupled with enzymatic digestion as well as several other methods known to those skilled in the art.

### Epitope Binding and Cross-Blocking Antibodies

An anti-canine IL-31RA antibody or antigen-binding fragment thereof of the present invention includes any antibody or antigen-binding fragment thereof that binds to the same epitope in canine IL-31RA as the one of the antibodies, disclosed herein, bind, e.g., such as the 218D9 antibody which binds to the epitope comprising the amino acid sequence either SEQ ID NO: 104, SEQ ID NO: 105, or both SEQ ID NO: 104 and SEQ ID NO: 105, or the 51F8 antibody which binds to the epitope comprising the amino acid sequence either SEQ ID NO: 98, SEQ ID NO: 100, or both SEQ ID NO: 98 and SEQ ID NO: 100, including caninized antibodies, and any antibody or antigen-binding fragment that cross-blocks (partially or fully) or is cross-blocked (partially or fully) by an antibody or fragment discussed herein for canine IL-31RA binding; as well as any variant thereof.

The cross-blocking antibodies and antigen-binding fragments can be identified based on their ability to cross-compete with e.g., the 218D9 or 51F8 antibody in standard binding assays (e.g., BIACore®, ELISA, as exemplified below, or flow cytometry). For example, standard ELISA assays can be used in which a recombinant canine IL-31RA protein is immobilized on the plate, one of the antibodies is fluorescently labeled and the ability of non-labeled antibodies to compete off the binding of the labeled antibody is evaluated. Additionally or alternatively, BIAcore® analysis can be used to assess the ability of the antibodies to cross-compete. The ability of a test antibody to inhibit the binding of the e.g., 51F8 or 218D9 antibody, to canine IL-31RA demonstrates that the test antibody can compete with the 51F8 or 218D9 antibody for binding to canine IL-31RA and thus, may, in some cases, bind to the same epitope on canine IL-31RA as the 51F8 and/or 218D9 antibody binds.

Antibodies and fragments thereof that bind to the same epitope as any of the anti-canine IL-31RA antibodies or fragments of the present invention also form part of the present invention.

### Pharmaceutical Compositions and Administration

To prepare pharmaceutical or sterile compositions comprising the antibodies of the present invention, these antibodies can be admixed with a pharmaceutically acceptable carrier or excipient. [See, e.g., Remington's Pharmaceutical Sciences and U.S. Pharmacopeia: National Formulary, Mack Publishing Company, Easton, PA (1984)].

Formulations of therapeutic and diagnostic agents may be prepared by mixing with acceptable carriers, excipients, or stabilizers in the form of, e.g., lyophilized powders, slurries, aqueous solutions or suspensions [see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY]. In one embodiment, the antibodies of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in canines. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In particular embodiments, the antibodies of the present invention can be administered by an invasive route such as by injection. In further embodiments of the invention, the antibodies of the present invention, or pharmaceutical composition thereof, is administered intravenously, subcutaneously, intramuscularly, intraarterially, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector. The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively, one may administer the antibodies of the present invention in a local rather than systemic manner, often in a depot or sustained release formulation.

The administration regimen depends on several factors, including the serum or tissue turnover rate of the therapeutic antibodies, the level of symptoms, the immunogenicity of the therapeutic antibodies and the accessibility of the target cells in the biological matrix. Preferably, the administration regimen delivers sufficient therapeutic antibodies to effect improvement in the target disease/condition state, while simultaneously minimizing undesired side effects. Accordingly, the amount of biologic delivered depends in part on the particular therapeutic antibodies and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, NY (1991); Bach (ed.) Monoclonal Antibodies and Peptide Therapy in Autoimmune Diseases, Marcel Dekker, New York, NY (1993); Baert, et al. New Engl. J Med. 348:601-608 (2003); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)].

Determination of the appropriate dose is made by the veterinarian, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of the symptoms.

Antibodies provided herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J. Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)]. Doses may also be provided to achieve a pre-determined target concentration of antibodies of the present invention in the canine's serum, such as 0.1, 0.3, 1, 3, 10, 30, 100, 300 μg/ml or more. In other embodiments, antibodies of the present invention is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject (e.g., a canine) with a disorder, condition and/or symptom, or with the potential to develop such a disorder, disease or symptom.

As used herein, the terms “therapeutically effective amount”, “therapeutically effective dose” and “effective amount” refer to an amount of antibodies of the present invention that, when administered alone or in combination with an additional therapeutic agent to a cell, tissue, or subject, e.g., canine, is effective to cause a measurable improvement in one or more symptoms of a disease or condition or the progression of such disease or condition. A therapeutically effective dose further refers to that amount of the antibodies sufficient to result in at least partial amelioration of symptoms, e.g., treatment, healing, prevention or amelioration of the relevant medical condition, or an increase in rate of treatment, healing, prevention or amelioration of such conditions. When applied to a combination, a therapeutically effective dose refers to combined amounts of the active ingredients that result in the therapeutic effect, whether administered in combination, serially, or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess severity of the condition.

### EXAMPLES

### Example 1

### IL-31 Receptor Alpha

### Nucleotide Sequence

The nucleotide sequence of SEQ ID NO: 1 encodes the extracellular domain of the canine IL-31 receptor alpha (cIL-31RA) fused to a HIS tag. Canine IL-31RA ECD HIS-tagged protein comprises the amino acid sequence of SEQ ID NO: 2. The nucleotide sequence was prepared by chemical synthesis and then cloned into expression plasmids that are suitable for production of the corresponding proteins in eukaryotic cells, either HEK-293 or CHO cells.

Canine IL-31RA ECD-10His:
[SEQ ID NO: 1]
gtgctgcccgccaagcccgagaacatcagctgcatcttctactacgagg
agaacttcacctgcacctggagccccgagaaggaggccagctacacctg
gtacaaggtgaagagaacctacagctacggctacaagagcgacatctgc
agcaccgacaacagcaccagaggcaaccacgccagctgcagcttcctgc
cccccaccatcaccaaccccgacaactacaccatccaggtggaggccca
gaacgccgacggcatcatgaagagcgacatcacctactggaacctggac
gccatcatgaagatcgagccccccgagatcttcagcgtgaagagcgtgc
tgggcatcaagagaatgctgcagatcaagtggatcagacccgtgctggc
cccccacagcagcaccctgaagtacaccctgagattcagaaccatcaac
agcgcctactggatggaggtgaacttcaccaaggaggacatcgacagag
acgagacctacaacctgaccgagctgcaggccttcaccgagtacgtgat
gaccctgagatgcgcccccgccgagagcatgttctggagcggctggagc
caggagaaggtgggcaccaccgaggaggaggccccctacggcctggacc
tgtggagagtgctgaagcccgccatggtggacggcagaagacccgtgca
gctgatgtggaagaaggccaccggcgcccccgtgctggagaaggccctg
ggctacaacatctggtacttccccgagaacaacaccaacctgaccgaga
ccgtgaacaccaccaaccagacccacgagctgtacctgggcggcaagac
ctactgggtgtacgtggtgagctacaacagcctgggcgagagccccgtg
gccaccctgagaatccccgccctgaacgagaagaccttccagtgcatcg
aggccatgcaggcctgcctgacccaggaccagctggtggtggagtggca
gagcagcgcccccgaggtggacacctggatggtggagtggttccccgac
gtggacagcgagcccagcagcttcagctgggagagcgtgagccaggcca
gaaactggaccatccagaaggacgagctgaagcccctgtggtgctacaa
catcagcgtgtaccccgtgctgagagacagagtgggccagccctacagc
acccaggcctacgtgcaggagggcatccccagcgccggccccgtgaccc
aggccgacagcatcggcgtgaagaccgtgaccatcacctggaaggagat
ccccaagagcaagagaaacggcttcatcaagaactacaccatcttctac
caggccgaggacggcaaggagttcagcaagaccgtgaacagcaacatcc
tgcagtacagactggagagcctgaccagaagaaccagctacagcctgca
ggtgatggccagcaccaacgccggcggcaccaacggcaccaagatcaac
ttcaagaccctgagcatcagccaccaccaccaccaccaccaccaccacc
ac

### Example 2

### Expression and Purification of IL-31 Receptor Alpha ECD

Plasmids comprising the nucleotide sequence of SEQ ID NO: 1 were transfected into HEK-293 or CHO cells using electroporation via the MaxCyte instrument as per the manufacturer's recommendation. Several days following transfection, the supernatants of transfected cells and un-transfected controls were harvested and spun down to remove cellular debris. IL-31RA with the HIS tag was purified from cell culture fluids by passing the clarified harvested fluid from transfected cells over nickel columns as per the manufacturer's recommendation. Purified proteins were quantified by measuring their absorbance of ultraviolet light at 280 nm.

Canine IL-31RA ECD-10His:
[SEQ ID NO: 2]
VLPAKPENISCIFYYEENFTCTWSPEKEASYTWYKVKRTYSYGYKSDIC
STDNSTRGNHASCSFLPPTITNPDNYTIQVEAQNADGIMKSDITYWNLD
AIMKIEPPEIFSVKSVLGIKRMLQIKWIRPVLAPHSSTLKYTLRFRTIN
SAYWMEVNFTKEDIDRDETYNLTELQAFTEYVMTLRCAPAESMFWSGWS
QEKVGTTEEEAPYGLDLWRVLKPAMVDGRRPVQLMWKKATGAPVLEKAL
GYNIWYFPENNTNLTETVNTTNQTHELYLGGKTYWVYVVSYNSLGESPV
ATLRIPALNEKTFQCIEAMQACLTQDQLVVEWQSSAPEVDTWMVEWFPD
VDSEPSSFSWESVSQARNWTIQKDELKPLWCYNISVYPVLRDRVGQPYS
TQAYVQEGIPSAGPVTQADSIGVKTVTITWKEIPKSKRNGFIKNYTIFY
QAEDGKEFSKTVNSNILQYRLESLTRRTSYSLQVMASTNAGGINGTKIN
FKTLSISHHHHHHHHHH

TABLE 1
CANINE IL-31RA EXTRACELLULAR DOMAIN -HIS TAG

| | SEQ ID NO: | Nucleic Acid | Amino Acid |
|---|---|---|---|
| canine IL-31RA ECD-10His | 1 | X | |
| canine IL-31RA ECD-10His | 2 | | X |

### Example 3

### Binding of Canine IL-31RA to Biotinylated Canine IL-31

### Protocol

1) Coat immunoplate(s) with IL-31RA proteins by diluting to 1 μg/mL in phosphate-buffered saline solution (PBS Add 100 μL/well. Incubate the plate(s) at 2-7° overnight.
2) Wash the plates 3 times with 275 μL/well of phosphate-buffered saline solution plus TWEEN 20 (PBST).
3) Block the plates with 200 μL/well of blocking buffer [1% nonfat dried milk (NFDM) in PBST] for 30-45 minutes at 36±2° C. with gentle shaking (120±20 RPM).
4) Wash the plates 3 times with 275 μL/well of PBST.
5) 3-fold dilute biotinylated IL-31 (at 10 μg/mL) in 1% NFDM in PBST on a dilution plate, and transfer 100 μL/well to the immunoplate(s). Incubate for 30-45 minutes at 36±2° C. with gentle shaking (120±20 RPM).
6) Wash the plates 3 times with 275 μL/well of PBST.
7) Dilute horse raddish peroxidase-Streptavidin (IRP-Streptavidin) to a final dilution of 1:1000 in 1% NFDM in PBST.
8) Add 100 μL/well of HRP-Streptavidin to the immunoplate(s) and incubate for 30-45 minutes at 36±2° C. with gentle shaking (120±20 RPM).
9) Wash the plates 3 times with 275 μL/well of PBST.
10) Combine equal volumes of pre-warmed TMP 2-Component substrate immediately before use.
11) Add 100 μL/well of prepared 3,3,55-tetramiethylbenzidine (TMP) substrate to the immunoplate(s) and incubate in the dark for 10 to 15 minutes at 36±2° C. with gentle shaking (120±20 RPM).
12) Stop the reaction by addition of 100 μL/well of 1 M H3PO4.
13) Read the plates using a microplate reader at a wavelength of 450 nm with a reference wavelength of 540 nm.

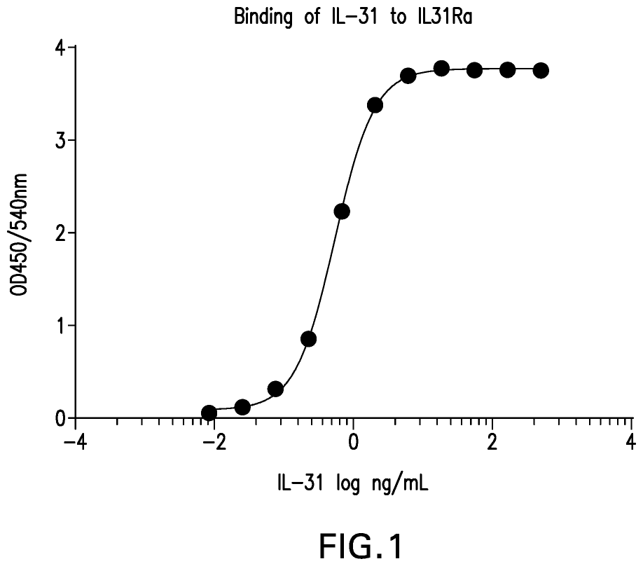

The extracellular domain (ECD) of canine IL-31RA was tested for its ability to bind to canine IL-31 (see, FIG. 1). The results indicate that IL-31RA ECD binds in a dose-dependent manner to biotinylated canine IL-31 with an EC50 of 0.55 ng/ml.

### Example 4

### Monoclonal Antibodies Against Canine IL-31 Receptor Alpha

Monoclonal antibodies (mAbs) against canine IL-31RA were produced by the immunization of mice multiple times with canine IL-31RA ECD. Mice were immunized via the Intraperitoneal route with IL-31RA ECD in GS proprietary adjuvant 3 times on days 0, 14, and 28 using 50 μg per mouse for first immunization and 25 μg per mouse for the subsequent boosts. On day 48 mice were immunized once more with 25 μg of antigen and 4 days later their spleen cells were fused with the myeloma SP2/0 cell line to produce hybridomas secreting antibodies. At various time points after immunization, sera were collected from mice and tested against canine IL-31RA by ELISA. The spleen cells from the mouse with highest IL-31RA ECD reactivity were fused with the myeloma SP2/0 cell line to produce hybridomas. Approximately 14 days after the fusion, supernatants from growing hybridomas were screened by flow cytometry using cells expressing the IL-31RA protein.

The reactivities of hybridoma were confirmed by ELISA as follows:

### Procedure for the ELISA:

1) Coat 96-well plates with IL-31RA (1 μg/mL in PBS buffer), 25 μL/well.
2) Incubate the plates at 4° C. overnight.
3) Wash the plates 3 times with PBST (PBS+0.05% Tween 20)
4) Block the plates with blocking buffer [PBS with 5% fetal bovine serum (FBS)], 25 μl/well for 30 minutes at room temperature.
5) Transfer 25 μl/well hybridoma supernatant to the 96-well plates, incubate 60 minutes at room temperature.
6) Wash the plates 3 times by PBST.
7) Add 25 μl/well anti-mouse HRP, 1:4000 dilution in blocking buffer, to the plates and incubate 60 minutes at room temperature.
8) Wash the plates 5 times by PBST.
9) Add TMB based reagent to the plates for colorimetric reaction for 2-3 minutes.
10) Stop the reactions with 0.16M sulfuric acid.
11) Read the plates by plate reader.

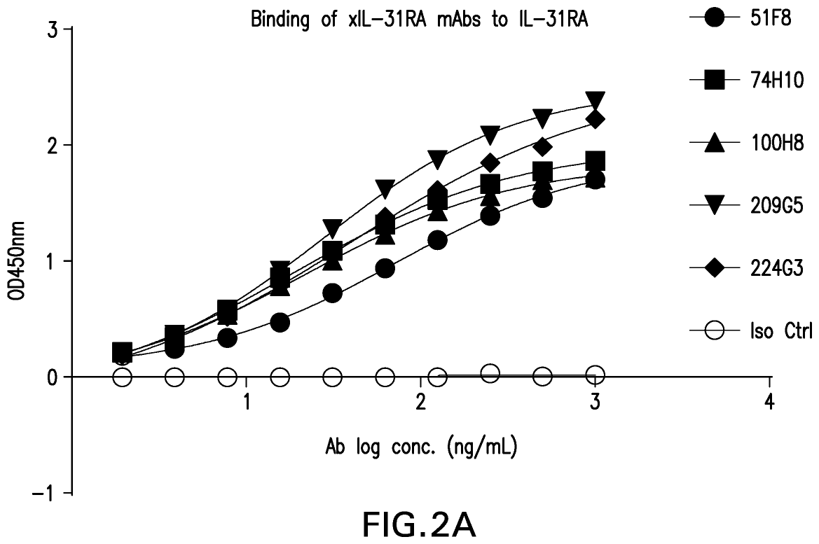

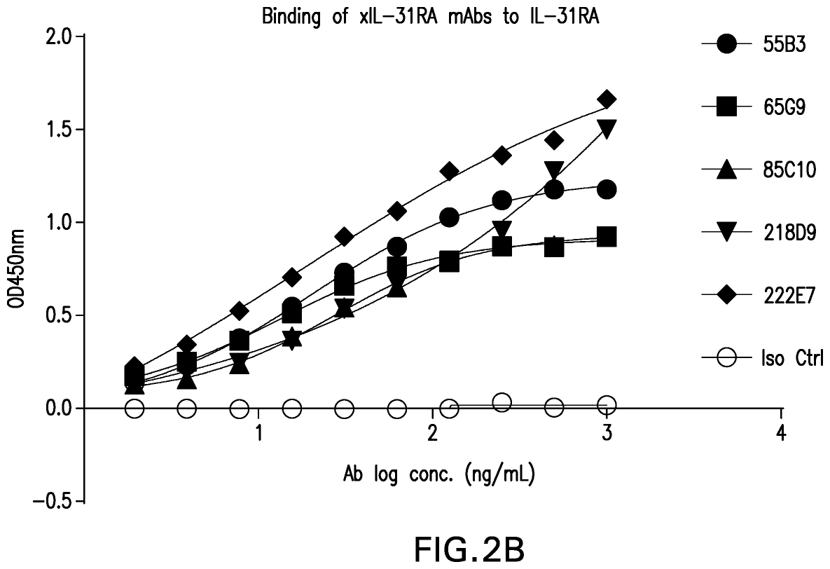

As shown in FIGS. 2A-2B, the selected mouse mAbs were tested for their reactivity to canine IL-31RA. The results indicate that the selected mouse mAbs bind to canine IL-31RA in a dose-dependent manner. Ten (10) mouse monoclonal antibodies were obtained that have strong binding reactivity to canine TL-3IRA, as shown in FIGS. 2A-2B.

The amino acid sequences of the heavy and light chain variable regions of these ten mouse monoclonal antibody are provided below.

100H8VH
[SEQ ID NO: 52]
EVQLQQSGPELVKPGASVKIPCKASGYTFTDYNMDWVKQSHGKSLEWIG
HINPNNGGILYNQKFKGKATLTVDRSSNTAYMELRSLTSEDTAVYFCAR
WAPLVRQPYWYFDVWGTGTTVTVSS
100H8VL
[SEQ ID NO: 53]
DIKMTQSPSSIFASLGERVTITCKASQDINSYLNWFQQKPGKSPKTLIY
RADRLVDGVPSRFSGSGSGQDYSLTISSLEYEDMGIYYCLQYDEFPLTF
GAGTKLELN
222E7VH
[SEQ ID NO: 54]
EVQLQQSGPELVKPGSSVKIPCKASGYTFTDYNMDWVKQSHGKSLEWIG
HINPNIGGTLYNQKFKGKATLSVDKSSSTAYMELRSLTSEDTAVYYCAR
WAQLQRQPYWYFDVWGPGTTVTVSS
222E7VL
[SEQ ID NO: 55]
DIKMTQSPSSMYASLGERVTITCKASQDINSYLNWFQQKPGKSPKTLIY
RVNRLVDGVPSRFSGSGSGQDYSLTISSLEYEDMGIYYCLQYDEFPLTF
GAGTKLELK
55B3VH
[SEQ ID NO: 56]
EVQLQQSGPELVKPGASVKIPCKASGYTFTDYNMDWVKQSHGKSLEWIG
HINPNTGGTIYNQKFKGKATLTVDKSSSTAYMDLRSLTSEDTAVYYCAR
WAQLLRQPYWYFDVWGTGTTVTVSS
55B3VL
[SEQ ID NO: 57]
DIKMTQSPSSMYASLGERVTITCKASQDINSYFNWVQQKPGKSPKTLIF
RANRLVDGVPSRFSGSGSGQDYSLTINSLEYEDMGIYYCLQYDEFPLTF
GAGTKLELK
74H10VH
[SEQ ID NO: 58]
EVQLQQSGPELVKPGASMKIPCKTSGYTFTDYNMDWVKQSHGKSLEWIG
HINPNNGGTLYNQKFKDKATLTVDRSSNTAYMELRSLTSEDTAVYYCAR
WAPLLRQPYWYFDVWGTGTTVTVSS
74H10VL
[SEQ ID NO: 59]
DIKMTQSPSSMYASLGERVTITCKASQDINSYLNWFQQKPGKSPKTLIY
RADRLVDGVPSRFSGSGSGQDYSLTISSLEYEDMGIYYCLQYDEFPLTF
GDGTKLELN
209G5VH
[SEQ ID NO: 60]
EVQLQQSVAELVRPGASVKLSCTASGFNIKNTYMHWVKERPEQGLEWIG
RIDPANGNSKYAPKFQGKATITTDTSSNTAYLQLSSLTSEDTAIYYCAR
YYYVSSHFDVWGTGTTVTVSS
209G5VL
[SEQ ID NO: 61]
SIVMTQTPKFLLVSAGDRVTITCKASQSVTNDVTWYQQKPGQSPKLLIY
YASNRYTGVPDRFTGSGYGTDFTFTISTVQAEDLAVYFCQQDYSSPFTF
GSGTKLEIK
51F8VH
[SEQ ID NO: 62]
EVQLQQSVAELVRPGASVKLSCTASGFNIKNTFIHWVKQRPEQGLEWIG
RIDPANGNTKYAPKFQGRATLTADTSSNTAYLQLSSLTSEDTAIYYCAR
YYYVSSYFDVWGTGTTVTVSS
51F8VL
[SEQ ID NO: 63]
SIVMTQTPKFLLVSAGDRVTITCKASQSVTNDVTWYQQKPGQSPKLLIF
SASNRYTGVPDRFTGSGYGTDFTFTISTVQAEDLAVYFCQQDYSSPWTF
GGGTKLEIK
65G9VH
[SEQ ID NO: 64]
EFQLQQSGPELVKPGASVKISCKASGYSFTDYGMNWLKQINGKSLEWIA
IINPNYGTASSNPKFKDKATLTVDHSSSTAYMQLSSLTSEDSAVYYCAR
AFDGYYFYWYFDVWGTGTTVTVSS
65G9VL
[SEQ ID NO: 65]
DVVMTQTPLSLPVSLGDQASISCRSSQSLIHTNGNTYLHWYLQKPGQSP
KLLIYKVSNRFSGVPDRFSGSGSGTDFTLKISRLEAEDLGVYFCSQSTH
VPLTFGAGTKLELR
85C10VH
[SEQ ID NO: 66]
EFQLQQSGPELVKPGASVKISCKASGYSFTDYSINWVKQSNGKSLEWIG
VINPNYGTSSHNQKFKGKATMTVDQSSSTAYMQLNSLTSEDSAVYYCAR
ALDDYYFYWYFDVWGIGTTVTVSS
85C10VL
[SEQ ID NO: 67]
DVVMTQTPLSLPVSLGDQASISCRSSQSLVHTNGNTYLHWYLQKPGQSP
KLLIYKVSNRFSGVPDRFSGSGSGTDFTLKISRVEAEDLGVYFCSQTTH
VPLTFGAGTKLELK
218D9VH
[SEQ ID NO: 68]
QVTLKESGPGILQPSQTLSLTCSFSGFSLSTFGRGVGWIRQPSGKGLDW
LTHIWWDDDKYYNPALKSRLTISKDTSKNQVFLKIANVDTADTATYYCA
RIAGGLRRAPYAMDSWGQGTSVTVSS
218D9VL
[SEQ ID NO: 69]
DIQMTQSPASLSVSVGETVTITCRASENIYSSLAWYQQKQGKSPQLLVY
AATNLADGVPSRFSGSGSGTQYSLKINSLQSEDSGNYYCQHFRDTPPTF
GGGTKLEIK
224G3VH
[SEQ ID NO: 70]
EVQLVESGGDLVKPGGSLKLSCAASGFTFSSYGMSWVRQTPDKRLEWVA
TISNGGSYTYYPDSVKGRFTISRDNAKNTLYMQMSSLKSEDTAMYYCAR
NEPPDYWGQGTSVTVSS
224G3VL
[SEQ ID NO: 71]
QIVLTQSPAIMSASLGERVTMTCTASSSVGSSYLHWYQQKPGSSPKLWI
YDTSNLASGVPVRFSGSGSGTSYSLTISSMEAEDAATYYCHQYHRSPYT
FGGGTKLEIK

Table 2 below, provides the association rate constant (ka), dissociation rate constant (kd), and dissociation constant (KD), as analyzed by Octet Kinetics (see also, Example 9 below). These constants reflect the binding affinity of the individual monoclonal antibodies for canine IL-31 RA.

TABLE 2
BINDING AFFINITY OF SELECTED
ANTI-IL-31RA ANTIBODIES

| mAb | KD (M) | ka (1/Ms) | kd (1/s) |
|---|---|---|---|
| 100H8 | 1.52E−10 | 7.88E+05 | 1.20E−04 |
| 51F8 | 2.07E−10 | 6.84E+05 | 1.41E−04 |
| 55B3 | 2.72E−10 | 5.68E+05 | 1.54E−04 |
| 65G9 | 4.51E−10 | 7.87E+05 | 3.55E−04 |
| 74H10 | 1.96E−10 | 4.63E+05 | 9.05E−05 |
| 209G5 | 1.31E−10 | 3.70E+05 | 4.87E−05 |
| 85C10 | 8.51E−10 | 4.27E+05 | 3.63E−04 |
| 218D9 | 6.98E−11 | 6.64E+05 | 4.63E−05 |
| 222E7 | 1.74E−10 | 1.62E+06 | 2.82E−04 |
| 224G3 | 9.64E−13 | 1.12E+06 | 1.08E−06 |

The results show that the selected mAbs have low nanomolar to sub-picomolar binding affinities ranging from about 0.85 nM to about 1 pM.

The sets of the six CDRs for each of the ten monoclonal antibodies described above are provided below in Table 3. In addition, the canonical structures for each of these CDRs are provided in Table 4 below.

TABLE 3
AMINO ACID SEQUENCES OF THE MOUSE CDRS

| | Amino Acid Sequence | SEQ ID NO: | Amino Acid Sequence | SEQ ID NO: |
|---|---|---|---|---|
| | 100H8 | | 74H10 | |
| HCDR1 | DYNMD | 3 | DYNMD | 3 |
| HCDR2 | HINPNNGGILYNQKFKG | 10 | HINPNNGGTLYNQKFKD | 11 |
| HCDR3 | WAPLVRQPYWY | 20 | WAPLLRQPYWYFDV | 21 |
| LCDR1 | KASQDINSYLN | 30 | KASQDINSYLN | 30 |
| LCDR2 | RADRLVD | 40 | RADRLVD | 40 |
| LCDR3 | LQYDEFPLT | 45 | LQYDEFPLT | 45 |
| | 222E7 | | 55B3 | |
| HCDR1 | DYNMD | 3 | DYNMD | 3 |
| HCDR2 | HINPNIGGTLYNQKFKG | 12 | HINPNTGGTIYNQKFKG | 13 |
| HCDR3 | WAQLQRQPYWYFDV | 22 | WAQLLRQPYWYFDV | 23 |
| LCDR1 | KASQDINSYLN | 30 | KASQDINSYFN | 31 |
| LCDR2 | RVNRLVD | 38 | RANRLVD | 39 |
| LCDR3 | LQYDEFPLT | 45 | LQYDEFPLT | 45 |
| | 51F8 | | 209G5 | |
| HCDR1 | NTFIH | 5 | NTYMH | 6 |
| HCDR2 | RIDPANGNTKYAPKFQG | 15 | RIDPANGNSKYAPKFQG | 16 |
| HCDR3 | YYYVSSY | 25 | YYYVSSHFDV | 26 |
| LCDR1 | KASQSVTNDVT | 32 | KASQSVTNDVT | 32 |
| LCDR2 | SASNRYT | 41 | YASNRYT | 42 |
| LCDR3 | QQDYSSPWT | 47 | QQDYSSPFT | 48 |
| | 65G9 | | 218D9 | |
| HCDR1 | DYGMN | 7 | TFGRGVG | 8 |
| HCDR2 | IINPNYGTASSNPKFKD | 17 | HIWWDDDKYYNPALKS | 18 |
| HCDR3 | AFDGYYFYWYFDV | 27 | IAGGLRRAPYAMDS | 28 |
| LCDR1 | RSSQSLIHTNGNTYLH | 34 | RASENIYSSLA | 35 |
| LCDR2 | KVSNRFS | 37 | AATNLAD | 43 |
| LCDR3 | SQSTHVPLT | 50 | QHFRDTPPT | 51 |
| | 224G3 | | 85C10 | |
| HCDR1 | SYGMS | 9 | DYSIN | 4 |
| HCDR2 | TISNGGSYTYYPDSVKG | 19 | VINPNYGTSSHNQKFKG | 14 |
| HCDR3 | NEPPDY | 29 | ALDDYYFYWY | 24 |
| LCDR1 | TASSSVGSSYLH | 36 | RSSQSLVHTNGNTYLH | 33 |
| LCDR2 | DTSNLAS | 44 | KVSNRFS | 37 |
| LCDR3 | HQYHRSPYT | 49 | SQTTHVPLT | 46 |

TABLE 4
CANONICAL STRUCTURE OF THE MOUSE CDRs

| | L1 | L2 | L3 | H1 | H2 | H3 |
|---|---|---|---|---|---|---|
| 100H8 | L1-2 | L2-1 | L3-1 | H1-1 | H2-2 | H3-11 |
| 85C10 | L1-4 | L2-1 | L3-1 | H1-1 | H2-2 | H3-10 |
| 51F8 | L1-2 | L2-1 | L3-1 | H1-1 | H2-2 | H3-7 |
| 222E7 | L1-2 | L2-1 | L3-1 | H1-1 | H2-2 | H3-14 |
| 55B3 | L1-2 | L2-1 | L3-1 | H1-1 | H2-2 | H3-14 |
| 209G5 | L1-2 | L2-1 | L3-1 | H1-1 | H2-2 | H3-10 |
| 65G9 | L1-4 | L2-1 | L3-1 | H1-1 | H2-2 | H3-14 |
| 218D9 | L1-2 | L2-1 | L3-1 | H1-1 | H2-4 | H3-14 |
| 224G3 | L1-2 | L2-1 | L3-1 | H1-1 | H2-4 | H3-6 |
| 74H10 | L1-2 | L2-1 | L3-1 | H1-1 | H2-2 | H3-14 |

Of these 10 sets of CDRS in Table 3 above, a particular group of four anti-canine IL-31RA antibodies that bind IL-31RA were identified as comprising a striking amino acid sequence similarity.

HEAVY CHAIN:

| AB | HCDR1 | SEQ ID | HCDR2 | SEQ ID |
|---|---|---|---|---|
| 100H8 | DYNMD | NO: 3 | HINPNNGGILYNQKFKG | NO: 10 |
| 74H10 | DYNMD | NO: 3 | HINPNNGGTLYNQKFKD | NO: 11 |
| 222E7 | DYNMD | NO: 3 | HINPNIGGTLYNQKFKG | NO: 12 |
| 55B3 | DYNMD | NO: 3 | HINPNTGGTIYNQKFKG | NO: 13 |
| AB | HCDR3 | | SEQ ID | |

| | | |
|---|---|---|
| 100H8 | WAPLVRQ | NO: 20 |
| | PYWY | |
| 74H10 | WAPLLRQ | NO: 21 |
| | PYWYFDV | |
| 222E7 | WAQLQRQ | NO: 22 |
| | PYWYFDV | |
| 55B3 | WAQLLRQ | NO: 23 |
| | PYWYFDV | |

LIGHT CHAIN:

| AB | LCDR1 | SEQ ID | LCDR2 | SEQ ID |
|---|---|---|---|---|
| 100H8 | KASQDINSYLN | NO: 30 | RADRLVD | NO: 40 |
| 74H10 | KASQDINSYLN | NO: 30 | RADRLVD | NO: 40 |
| 222E7 | KASQDINSYLN | NO: 30 | RVNRLVD | NO: 38 |
| 55B3 | KASQDINSYFN | NO: 31 | RANRLVD | NO: 39 |
| AB | LCDR3 | SEQ ID | | |
| 100H8 | LQYDEFPLT | NO: 45 | | |
| 74H10 | LQYDEFPLT | NO: 45 | | |
| 222E7 | LQYDEFPLT | NO: 45 | | |
| 55B3 | LQYDEFPLT | NO: 45 | | |

Indeed, all of the HCDR1's in this group of four antibodies have the identical amino acid sequence (SEQ ID NO: 3). Whereas the four HCDR2's differ, they do not differ much. Notably, the HCDR2 of 100H8 differs from the three other HCDR2's by having an isoleucine residue at the ninth position rather than a threonine residue. 74H10 further differs from 100H8 by possessing an aspartic acid residue at its C-terminus rather than a glycine residue. Like 100H8, the other three HCDR2's have a glycine residue at their C-terminus. Unlike 100H8, the other three HCDR's have three additional amino acid residues (FDV) at their C-terminus. Both 74H10 and 55B3 have a leucine at their fourth position, rather than a valine residue like 100H8, whereas 222E7 has a glutamine residue. Finally, whereas both 100H8 and 74H10 have a proline residue at the third position, both 222E7 and 55B3 have a glutamine residue at the third position.

Three of the four LCDR1's in this group of four antibodies have the identical amino acid sequence (SEQ ID NO: 30), but whereas the LCDR1 of 55B3 shares their first four amino acid residues, its LCDR1 differs from the other three LCDR1s in its remaining seven amino acid residues. The LCDR2 of 100H8 and 74H10 have identical amino acid sequences (SEQ ID NO: 40), but the LCDR2 of 55B3 and 222E7 both differ from the other two LCDR2s by having an asparagine residue at position three instead of the aspartic acid residue of 100H8 and 74H10. In addition, the LCDR2 of 222E7 has a valine residue at position two rather than an alanine residue found in the LCDR2 of the three other antibodies. Notably, all four LCDR3's in this group of four antibodies have the identical amino acid sequence (SEQ ID NO: 45).

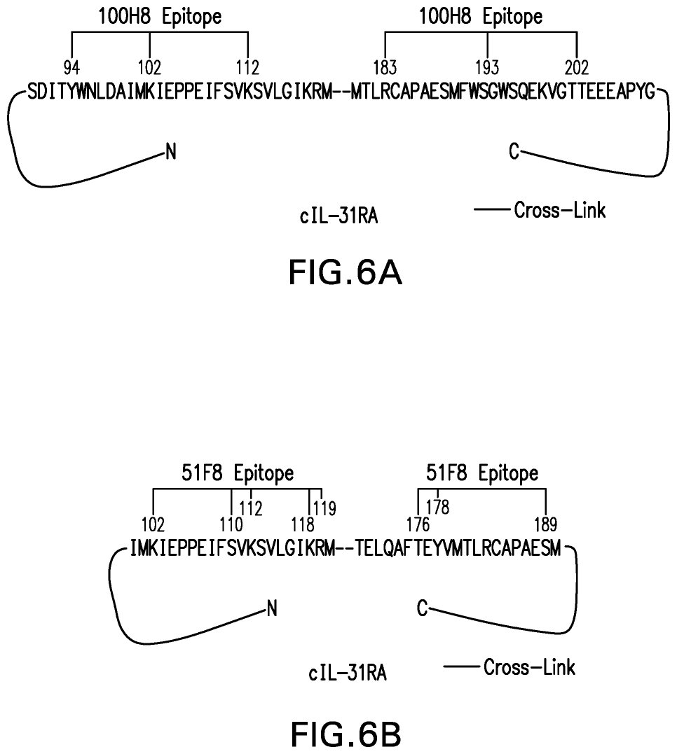

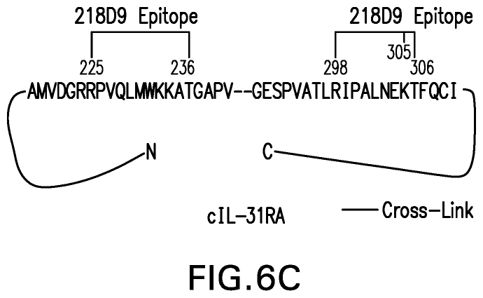

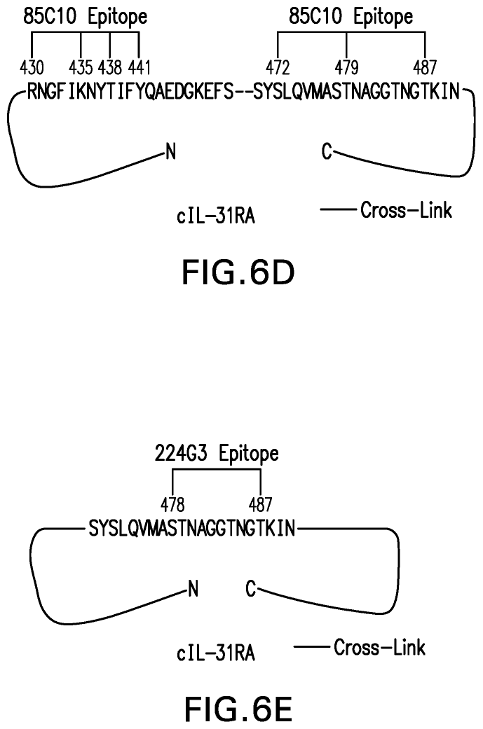

The remaining six (6) antibodies to canine IL-31RA detailed above, i.e., 65G9, 85C10, 224G3, 51F8, 209G5, and 218D9, can be further broken down into two pairs of antibodies that have noticeable identity in their respective CDR amino acid sequences, and two antibodies that are relative outliers. Accordingly, there is appreciable amino acid sequence identity between the sets of 6CDRs of antibody 65G9 with that of antibody 85C10 (see, Table 3). Consistently, antibodies 65G9 and 85C10 both bind two linear sequences in the C-terminal region of IL-31RA-ECD, one of which is the same (SEQ ID NO: 106; see, Table 6 and FIG. 6D) and the other one has substantial overlap (compare SEQ ID NO: 107 with SEQ ID NO: 108; see, Table 6). Notably, one of the outliers, antibody 244G3, binds to an epitope comprising a single linear sequence in the C-terminal region of IL-31RA-ECD (see, FIG. 6E), which is contained with the second linear sequence of antibody 85C10 (compare SEQ ID NO: 109 with SEQ ID NO: 108; see, Table 6).

The second group contains antibodies 51F8 and 209G5, which also have appreciable amino acid sequence identity between their respective sets of 6CDRs (see, Table 3). Consistently, antibodies 51F8 and 209G5 both bind two linear sequences in the N-terminal region of IL-31RA-ECD, one of which is the same (SEQ ID NO: 98; see, Table 6 and FIG. 6B), whereas the second linear sequence of the IL-31RA-ECD that antibody 209G5 binds (SEQ ID NO: 101) is within the amino sequence of the second linear sequence of IL-31RA-ECD that antibody 51F8 binds (SEQ ID NO: 100; see, Table 6).

The other outlier, antibody 218D9 binds to two linear amino acid sequences located in the middle portion of the amino acid sequence of IL-31RA-ECD (SEQ ID NOs: 104 and 105; see, FIG. 6C and Table 6). This antibody proved to be both a strong binder of IL-31RA and a good blocker of the binding of IL-31RA with IL-31.

### Example 5

### Blocking Activity of Anti-IL-31 Receptor Alpha Antibodies

The ability of anti-canine IL-31RA hybridoma supernatants to block the binding of IL-31 to IL-31RA were evaluated in the blocking ELISA described below.

### Protocol

1) Coat 96-well half area plates with IL-31RA (1 μg/mL in PBS buffer), 25 μL/well.
2) Incubate the plates at 4° C. overnight.
3) Wash the plates 3 times by PBST (PBS+0.05% Tween 20)
4) Block the plates with blocking buffer (PBS with 5% FBS), 25ul/well, for 30 minutes at room temperature.
5) Transfer 25 ul/well hybridoma supernatant to the 96-well plates, incubate 60 minutes at room temperature.
6) Wash the plates 3 times with PBST.
7) Transfer 25 μL/well of biotinylated IL31 (0.5 μg/mL in blocking buffer,) incubate 60 minutes at room temperature.
8) Wash the plates 3 times with PBST.
9) Add 25 l/well Streptavidin-IRP, 1:5000 dilution in blocking buffer, to the plates and incubate 60 minutes at room temperature.
10) Wash the plates five times with PBST.
11) Add TMB based reagent to the plates for colorimetric reaction for 2-3 minutes.
12) Stop the reactions with 0.16 M sulfuric acid.
13) Read the plates by plate reader.

### Results:

Out of the approximately 2000 hybridoma clones initially identified, approximately 300 were found to have binding affinity for IL-31RA, and about 10 of such clones also showed significant blocking of the binding of canine IL-31 to canine IL-31RA (see, Examples below).

### Example 6

### FACS Assay for Testing Blocking Activity of Monoclonal Antibodies Against Canine IL31-RA

In order to develop a cell-based assay to assess binding and blocking of canine IL-31 by anti-canine IL-31RA antibodies, the nucleotide sequences of the canine IL-31RA with c-terminal Flag tag and OSMR with c-terminal HA tag were prepared by chemical synthesis and then cloned into lentivirus vector Lenti-puro and Lenti-Hygro, respectively. The lentivirus Lenti-puro-IL31RA-Flag and Lenti-Hygro-OSMR-HA prepared from the Lenti-X 293T cells were co-transfected into CHO-kl cells. The CHO stable cell pool co-expressing canine IL-31RA and OSMR was selected by FACS with anti-flag and anti-HA antibodies. Single cell clones were isolated from the stable pool. The developed CHO-IL-31RA/OSMR table cell line is applied to screen anti-canine IL-31RA monoclonal antibodies for blocking of IL-31 with its receptor complex IL-31RA/OSMR.

### Materials

Cell line: CHO-IL-31RA/OSMR stable cell line
Cell growth medium: F-12K Medium with 10% FBS, 8 μg/ml Puromycin and 200 μg/ml hygromycin
Recombinant canine IL-31-His protein (0.5 μg/ml)
FACS Buffer: PBS
Isotype control: Mouse IgG (Genscript,A01007) (3 μg/ml)
Secondary antibody: Mouse anti-His tag antibody, 1 μg/ml (Genscript, A01802)
Flow cytometer: BD FACSCanto

### Flow Cytometry Procedure

1) CHO-IL-31RA/OSMR cells were grown in the growth medium in T75 flask.
2) Trypsinize to detach the cells, and then resuspend the cells in 5 mL fresh growth medium.
3) Spin down the cells at 300 g for 3 min, discard the supernatant and wash the cells twice with PBS.
4) Resuspend the cells in PBS as 2×106 cells/mL.
5) The cells were plated into 96-well assay plate with 50 μl/well.
6) Mix anti-IL-31RA antibody (20 μg/mL) or isotype control with IL-31 (1 μg/ml), and then transfer 50 μl of the mixture into each well of the assay plate.
7) After incubation at 4° C. for 40 min, the cells were washed twice by 150 μl of cold PBS.
8) Add 100 μl of the secondary antibody (1 μg/mL) into each well of the assay plate.
9) After incubation at 4° C. for 40 min, the cells were washed twice by 150 μl of cold PBS.
10) Resuspend the cells in 100 μp/well cold PBS and read by the Flow cytometry.

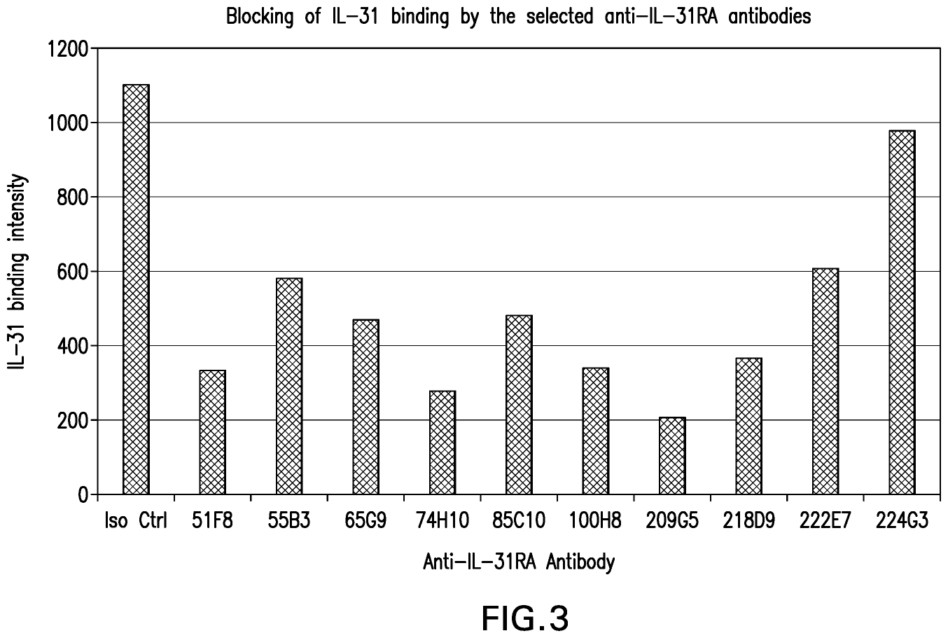

As shown in FIG. 3, the FACS result indicate that nine of the ten mouse anti-canine IL-31RA mAbs can significantly block the binding of IL-31 with the IL-31RA/OSMR complex presented on the CHO-IL-31RA/OSMR cells. The lead antibodies are 51F8, 74H10, 100H8, 209G5 and 218D9.

### Example 7

### STAT-3 Assay

Stat-3 is known to be activated by IL-31 in cells comprising the the heterodimeric receptor for IL-31. In order to develop an assay to assess the activation of STAT-3 by canine IL-31, the nucleotide sequences encoding IL-31RA and OSMR, respectively, were prepared by chemical synthesis and then cloned into expression vectors pcDNA3.1. The vectors containing the IL-31RA and OSMR nucleotide sequences, respectively, were co-transfected into Ba/f3 cells and the transfected cells, denoted as “Ba/f3-OI”, were grown as a pool under antibiotic selection. The ability of canine IL-31 to induce STAT-3 activation was tested as follows.

### Materials:

Cell line: Ba/f3-OI stable pool cells
Growth medium with mouse IL-3 or with canine IL-31 (cIL-31):
RPMI 1640 435 ml (ThermoFisher, 12633-020)
FBS 50 mL (SAFC cat #12003c-500 mL)
2-Mercaptoethanol (50 mM) 0.5 mL (Gibco 31350-010)
100×Pen Strep 5 mL (Gibco 15140-122 Lot1734040)
200 mM L-Glu 10 ml (Gibco 25030-081 Lot1677185)
500 ng/mL Geneticin G418 (from Gibco or Sigma)
5 ng/mL mIL-3 or 100 ng/mL cIL-31
Starvation medium: the growth medium without mIL-3 and cIL-31
p-STAT3 (Tyr705) Assay Kit: PerkinElmer, ALSU-PST3-A-HV

### Procedure:

### Cell Culture

1) Thaw a vial of the Ba/f3-OI cells, and grow the cells in the growth medium with mIL-3 in 37° C. C02 shaker with 125 rpm.
2). Passage the cells 2-3 passages to have the cells with ≥90% viability before set a cell-based assay.
3) To setup assay, harvest and resuspend the cells in the starvation medium to 1×10⁷ viable cells/mL.
4) Dispense cells into 96-well plate, 50 μL/well (about 5×10⁵ cells/well).
5) Three-fold dilute cIL-31 in starvation medium in a dilution plate, and then transfer 50 μL of each of the serial diluted cIL-31 aliquots into the cell plate.
6) Incubate the cell plate for 15-30 min in 37° C. CO₂ shaker with 125 rpm for 1-2 hrs.

### AlphaLISA Assay as Per Manufacturer's Instruction:

7) Spin down the cells, aspirate the supernatant, and add 1× lysis buffer of 50-100 μL/well. Incubate at RT for 10 min with 1000 rpm shaking.
8) Remove 30 μL of the cell lysate into a ½ area plate or freeze and store at −80° C. for future test.

### SUREFIRE Assay

9) Add 15 μL/well acceptor mix to the cell lysate. Seal and agitate plate for 2 min at 1000 rpm and then incubate for 1-2 hours at RT.
10) Add 15 μL/well donor mix to the cell lysate. Seal and agitate for 2 min at 1000 rpm, and then incubate for 1-2 hours at RT (the plate can be stored at 4° C. overnight. Incubate at room temp for 1 hr before reading the plate next day)
11) Read the plate on Alpha plate reader at 520-620 nm.

### Results:

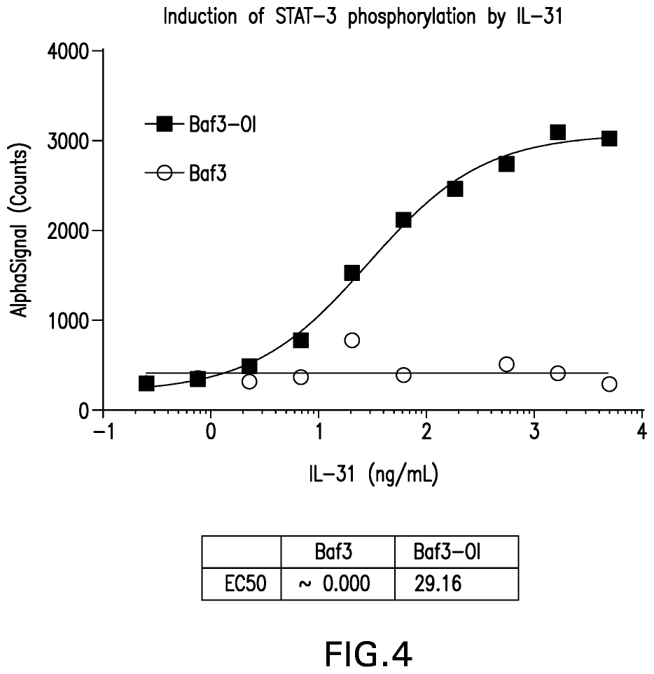

FIG. 4 shows the induction of STAT-3 phosphorylation by canine IL-31, which stimulates activation of STAT-3 in Ba/f3-OI cells in a dose dependent manner. Ba/f3 cells were used as the control. Ba/f3-OI cells expressing the IL-31 receptor complex were tested for IL-31-induced STAT-3 phosphorylation. The results indicate that STAT-3 phosphorylation was induced by IL-31 in the Baf3-OI cells in a dose-dependent manner, implying that: (i) the canine IL-31 receptor complex is successfully expressed on the cell surface; (ii) that the binding of canine IL-31 to the IL-31 receptor can stimulate the endogenous STAT3 phosphorylation; and (iii) then initiate its downstream signaling pathway.

### Example 8

### Biological Activity of Anti-Canine IL-31RA Antibodies

The ability of the anti-canine IL-31RA mAbs to inhibit the activation of STAT-3 in Ba/f3-OI cells is assessed as follows:
1) Thaw a vial of the Ba/f3-OI cells, and grow the Ba/f3-OI cells in the growth medium with mIL-3 in 37° C. C02 shaker with 125 rpm.
2) Passage the cells 2-3 passages to have the cells with ≥90% viability before set a cell-based assay.
3). To setup assay, harvest and resuspend the cells in the starvation medium to 1×10⁷ viable cells/mL.
4). Dispense cells into 96-well plate, 50 μL/well (about 5×10⁵ cells/well).
5) Three-fold dilute the antibody in starvation medium in a row on a 96-well plate, starting concentration at 200 μg/mL. Then add 5 μL cIL-31 in each well to get final concentration of 100 ng/mL.
6) Transfer 50 μL of the diluted antibody and cIL-31 mix into each well of the cell plate, gently mix.
7) Incubate the cell plate in 37° C. C02 shaker with 125 rpm for 15-30 min.

### AlphaLISA Assay as Per Manufacturer's Instruction: (Refer to Example 7)

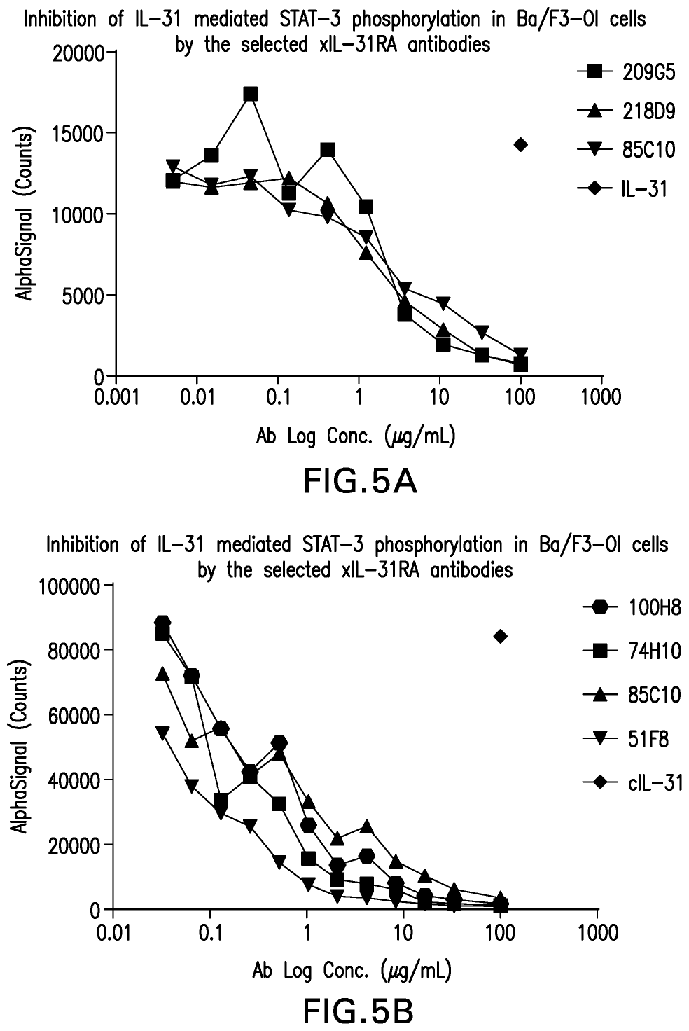

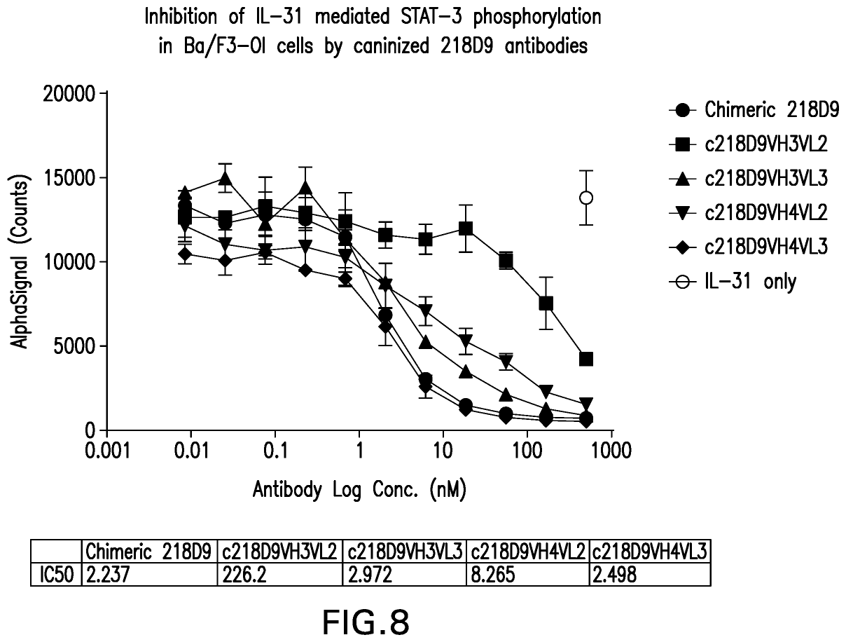

As exemplified in FIG. 5A for monoclonal antibodies 209G5, 218D9, and 85C10, in FIG. 5B for monoclonal antibodies 100H8, 74H10, 51F8, and 85C10, and again in FIG. 8 for various constructs of 218D9, all of the IL-31RA mAbs tested inhibit the canine IL-31 mediated STAT-3 phosphorylation in the Ba/f3-OI cells, whereas in the absence of these antibodies, there is no inhibition (labled IL-31). From FIG. 8, the IC50 for the various 218D9 antibody constructs was calculated to be approximately: 2.2 nM for the chimeric antibody; 230 nM for c218D9VH3VL2; 8.3 nM for c218D9VH4VL2; 2.9 nM for c218D9VH4VL3; and 2.5 nM for c218D9VH3VL3.

### Example 9

### In Vitro Binding of Anti-Canine IL-31RA Monoclonal Antibodies to Canine IL-31RA Receptor

All kinetics measurements were performed by Octet HTX using SA biosensors and Data Acquisition 12.0 software. A biotin-labeled antigen (canine IL-31RA) was loaded onto the pre-rehydrated SA biosensors for 120s at a concentration of 1 μg/mL. Next, the biosensors were placed into Octet Kinetics Buffer (PBS+0.02% Tween 20, 0.1% BSA) for the blocking phase for 120s. Then, for the association phase, antigen loaded biosensors were placed into 2-fold serial dilutions from 100 nM down to 3.13 nM of anti-IL-31RA monoclonal antibody in Octet Kinetics Buffer for 300s. The last well was buffer alone and that sensor was used for reference sensor subtraction. Finally, the biosensors were placed into Octet Kinetics Buffer for the dissociation phase for 300s. Analysis was performed using Data Analysis 12.0 software and curves were fitted using a 1:1 binding model.

Binding affinity measurement results indicate that all the tested monoclonal antibodies have low nanomolar to sub-picomolar binding affinities ranging from about 1.5 nM to about 1 pM (see, Table 2 above). Two of the top blocking antibodies: 51F8 and 218D9, had KDs of about 0.2 nM and about 0.07 nM respectively (see, Table 5B below).

### Example 10

### Caninized Antibodies

The DNA and protein sequence of the canine heavy and light chains are known and can be obtained by searching of the NCBI gene and protein databases. As indicated above, for canine antibodies there are four known IgG subtypes: IgG-A, IgG-B, IgG-C, and IgG-D, and two types of light chains, i.e., kappa and lambda. Without being bound by any specific approach, the process of producing caninized heavy and light chains that can be mixed in different combinations to produce caninized anti-canine IL-31 receptor alpha mAbs involves the following scheme:
i) Identify the DNA sequence of VH and VL domains comprising the CDRs of desired anti-IL-31 receptor alpha mAbs
ii) Identify the H and L chain CDRs of desired anti-IL-31RA mAbs
iii) Identify a suitable sequence for H and L chain of canine IgG
iv) Identify the DNA sequence encoding the endogenous CDRs of canine IgG H and L chains of the above sequence.
v) Replace the DNA sequence encoding endogenous canine H and L chain CDRs with DNA sequences encoding the desired anti-IL-31RA CDRs. In addition, optionally replace some canine framework residues with selected residues from the desired anti-IL-31 receptor alpha mAb framework regions.
vi) Synthesize the DNA from step (v), clone it into a suitable expression plasmid, and transfect the plasmids containing desired caninized H and L chains into HEK 293 cells.
vii) Purify expressed caninized antibody from HEK 293 supernatant.
vii) Test purified caninized antibody for binding to canine IL-31 receptor alpha chain.

The application of the above outlined steps can result in a set of caninized H and L chain amino acid sequences provided below.

c51F8VH1-cIgGBm
[SEQ ID NO: 72]
EVQLVQSGAEVKKPGASVKVSCKTSGYTFINTFIHWVRQAPGAGLDWMGQIDPANGNTKYAP
KFQGRVTLTADTSTSTAYMELSSLRAGDIAVYYCARYYYVSSYFDVWGQGTLVTVSSASTTA
PSVFPLAPSCGSTSGSTVALACLVSGYFPEPVTVSWNSGSLTSGVHTFPSVLQSSGLYSLSS
MVTVPSSRWPSETFTCNVAHPASKTKVDKPVPKRENGRVPRPPDCPKCPAPEMLGGPSVFIF
PPKPKDTLLIARTPEVTCVVVALDPEDPEVQISWFVDGKQMQTAKTQPREEQFAGTYRVVSV
LPIGHQDWLKGKQFTCKVNNKALPSPIERTISKARGQAHQPSVYVLPPSREELSKNTVSLTC
LIKDFFPPDIDVEWQSNGQQEPESKYRTTPPQLDEDGSYFLYSKLSVDKSRWQRGDTFICAV
MHEALHNHYTQESLSHSPGK
c51F8VH2-cIgGBm
[SEQ ID NO: 73]
EVQLVQSGAEVKKPGASVKVSCTASGFNIKNTFIHWVRQAPGAGLDWIGRIDPANGNTKYAP
KFQGRVTLTADTSTSTAYMELSSLRAGDIAVYYCARYYYVSSYFDVWGQGTLVTVSSASTTA
PSVFPLAPSCGSTSGSTVALACLVSGYFPEPVTVSWNSGSLTSGVHTFPSVLQSSGLYSLSS
MVTVPSSRWPSETFTCNVAHPASKTKVDKPVPKRENGRVPRPPDCPKCPAPEMLGGPSVFIF
PPKPKDTLLIARTPEVTCVVVALDPEDPEVQISWFVDGKQMQTAKTQPREEQFAGTYRVVSV
LPIGHQDWLKGKQFTCKVNNKALPSPIERTISKARGQAHQPSVYVLPPSREELSKNTVSLTC
LIKDFFPPDIDVEWQSNGQQEPESKYRTTPPQLDEDGSYFLYSKLSVDKSRWQRGDTFICAV
MHEALHNHYTQESLSHSPGK
c51F8VH3-cIgGBm
[SEQ ID NO: 74]
EVQLVQSGAEVKKPGASVKVSCTASGFNIKNTFIHWVRQAPGAGLDWIGRIDPANGNTKYAP
KFQGRATLTADTSTNTAYMQLSSLRAGDIAVYYCARYYYVSSYFDVWGQGTLVTVSSASTTA
PSVFPLAPSCGSTSGSTVALACLVSGYFPEPVTVSWNSGSLTSGVHTFPSVLQSSGLYSLSS
MVTVPSSRWPSETFTCNVAHPASKTKVDKPVPKRENGRVPRPPDCPKCPAPEMLGGPSVFIF
PPKPKDTLLIARTPEVTCVVVALDPEDPEVQISWFVDGKQMQTAKTQPREEQFAGTYRVVSV
LPIGHQDWLKGKQFTCKVNNKALPSPIERTISKARGQAHQPSVYVLPPSREELSKNTVSLTC
LIKDFFPPDIDVEWQSNGQQEPESKYRTTPPQLDEDGSYFLYSKLSVDKSRWQRGDTFICAV
MHEALHNHYTQESLSHSPGK
c51F8VH4-cIgGBm
[SEQ ID NO: 75]
EVQLVQSGAEVVKPGASVKVSCTASGFNIKNTFIHWVRQRPGAGLDWIGRIDPANGNTKYAP
KFQGRATLTADTSTNTAYMQLSSLRAGDIAVYYCARYYYVSSYFDVWGTGTLVTVSSASTTA
PSVFPLAPSCGSTSGSTVALACLVSGYFPEPVTVSWNSGSLTSGVHTFPSVLQSSGLYSLSS
MVTVPSSRWPSETFTCNVAHPASKTKVDKPVPKRENGRVPRPPDCPKCPAPEMLGGPSVFIF
PPKPKDTLLIARTPEVTCVVVALDPEDPEVQISWFVDGKQMQTAKTQPREEQFAGTYRVVSV
LPIGHQDWLKGKQFTCKVNNKALPSPIERTISKARGQAHQPSVYVLPPSREELSKNTVSLTC
LIKDFFPPDIDVEWQSNGQQEPESKYRTTPPQLDEDGSYFLYSKLSVDKSRWQRGDTFICAV
MHEALHNHYTQESLSHSPGKRNDAQPAVYLFQPSPDQLHTGSASVVCLLNSFYPKDINVKWK
VDGVIQDTGIQESVTEQDKDSTYSLSSTLTMSSTEYLSHELYSCEITHKSLPSTLIKSFQRS
ECQRVD
c51F8VL1-cCK
[SEQ ID NO: 76]
EIVMTQSPASLSLSQEEKVTITCKASQSVTNDVTWYQQKPGQAPKLLIYSASNRYTGVPSRF
SGSGSGTDFSFTISSLEPEDVAVYYCQQDYSSPWTFGQGTKLEIKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c51F8VL6-cCK
[SEQ ID NO: 77]
DIVMTQTPLSLSVSPGETASISCKASQSVTNDVTWFRQKPGQSPQLLIYSASNRYTGVPDRF
SGSGSGTDFTLRISTVEADDTGVYYCQQDYSSPWTFGQGTKLEIKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c51F8VL7-cCK
[SEQ ID NO: 78]
DIVMTQTPLSLSVSPGETASISCKASQSVTNDVTWFRQKPGQSPKLLIYSASNRYTGVPDRF
SGSGSGTDFTLRISTVEADDTGVYYCQQDYSSPWTFGQGTKLEIKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c100H8VH4-cIgGBm
[SEQ ID NO: 79]
EVQLVQSGAEVKKPGASVKVSCKTSGYTFTDYNMDWVRQAPGAGLDWMGHINPNNGGILYNQ
KFKGRVTLTADTSTSTAYMELSSLRAGDIAVYYCARWAPLVRQPYWYFDVWGQGTLVTVSSA
STTAPSVFPLAPSCGSTSGSTVALACLVSGYFPEPVTVSWNSGSLTSGVHTFPSVLQSSGLY
SLSSMVTVPSSRWPSETFTCNVAHPASKTKVDKPVPKRENGRVPRPPDCPKCPAPEMLGGPS
VFIFPPKPKDTLLIARTPEVTCVVVALDPEDPEVQISWFVDGKQMQTAKTQPREEQFAGTYR
VVSVLPIGHQDWLKGKQFTCKVNNKALPSPIERTISKARGQAHQPSVYVLPPSREELSKNTV
SLTCLIKDFFPPDIDVEWQSNGQQEPESKYRTTPPQLDEDGSYFLYSKLSVDKSRWQRGDTF
ICAVMHEALHNHYTQESLSHSPGK
c100H8VH7-cIgGBm
[SEQ ID NO: 80]
EVQLVQSGAEVKKPGASVKVSCKASGYTFTDYNMDWVRQAPGAGLDWIGHINPNNGGILYNQ
KFKGKATLTADTSTSTAYMELSSLRAGDIAVYYCARWAPLVRQPYWYFDVWGQGTLVTVSSA
STTAPSVFPLAPSCGSTSGSTVALACLVSGYFPEPVTVSWNSGSLTSGVHTFPSVLQSSGLY
SLSSMVTVPSSRWPSETFTCNVAHPASKTKVDKPVPKRENGRVPRPPDCPKCPAPEMLGGPS
VFIFPPKPKDTLLIARTPEVTCVVVALDPEDPEVQISWFVDGKQMQTAKTQPREEQFAGTYR
VVSVLPIGHQDWLKGKQFTCKVNNKALPSPIERTISKARGQAHQPSVYVLPPSREELSKNTV
SLTCLIKDFFPPDIDVEWQSNGQQEPESKYRTTPPQLDEDGSYFLYSKLSVDKSRWQRGDTF
ICAVMHEALHNHYTQESLSHSPGK
c100H8VH8-cIgGBm
[SEQ ID NO: 81]
EVQLVQSGAEVKKPGASVKVSCKASGYTFTDYNMDWVRQAPGAGLDWIGHINPNNGGILYNQ
KFKGKATLTVDRSTNTAYMELSSLRAGDIAVYFCARWAPLVRQPYWYFDVWGQGTLVTVSSA
STTAPSVFPLAPSCGSTSGSTVALACLVSGYFPEPVTVSWNSGSLTSGVHTFPSVLQSSGLY
SLSSMVTVPSSRWPSETFTCNVAHPASKTKVDKPVPKRENGRVPRPPDCPKCPAPEMLGGPS
VFIFPPKPKDTLLIARTPEVTCVVVALDPEDPEVQISWFVDGKQMQTAKTQPREEQFAGTYR
VVSVLPIGHQDWLKGKQFTCKVNNKALPSPIERTISKARGQAHQPSVYVLPPSREELSKNTV
SLTCLIKDFFPPDIDVEWQSNGQQEPESKYRTTPPQLDEDGSYFLYSKLSVDKSRWQRGDTF
ICAVMHEALHNHYTQESLSHSPGK
c100H8VL1-cCK
[SEQ ID NO: 82]
EIVMTQSPASLSLSQEEKVTITCKASQDINSYLNWYQQKPGQAPKLLIYRADRLVDGVPSRF
SGSGSGTDFSFTISSLEPEDVAVYYCLQYDEFPLTFGAGTKVELKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c100H8VL2-cCK
[SEQ ID NO: 83]
EIVMTQSPASLSLSQEEKVTITCKASQDINSYLNWFQQKPGQAPKLLIYRADRLVDGVPSRF
SGSGSGTDFSFTISSLEPEDVAVYYCLQYDEFPLTFGAGTKLELKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c100H8VL3-cCK
[SEQ ID NO: 84]
DIKMTQSPASLSLSQEEKVTITCKASQDINSYLNWFQQKPGQAPKLLIYRADRLVDGVPSRF
SGSGSGTDFSFTISSLEPEDVAVYYCLQYDEFPLTFGAGTKLELKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c100H8VL4-cCK
[SEQ ID NO: 85]
DIVMTQTPLSLSVSPGETASISCKASQDINSYLNWFRQKPGQSPQLLIYRADRLVDGVPDRF
SGSGSGTDFTLRISTVEADDTGVYYCLQYDEFPLTFGAGTKVELKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c218D9VH1-cIgGBm
[SEQ ID NO: 86]
ELTLQESGPGLVKPSQTLSLTCVVSGGSVTTFGRGVGWIRQRPGRGLEWMGHIWWDDDKYYN
PALKSRISITADTAKNQFSLQLSSMTTEDTAVYYCARIAGGLRRAPYAMDSWGQGTLVTVSS
ASTTAPSVFPLAPSCGSTSGSTVALACLVSGYFPEPVTVSWNSGSLTSGVHTFPSVLQSSGL
YSLSSMVTVPSSRWPSETFTCNVAHPASKTKVDKPVPKRENGRVPRPPDCPKCPAPEMLGGP
SVFIFPPKPKDTLLIARTPEVTCVVVALDPEDPEVQISWFVDGKQMQTAKTQPREEQFAGTY
RVVSVLPIGHQDWLKGKQFTCKVNNKALPSPIERTISKARGQAHQPSVYVLPPSREELSKNT
VSLTCLIKDFFPPDIDVEWQSNGQQEPESKYRTTPPQLDEDGSYFLYSKLSVDKSRWQRGDT
FICAVMHEALHNHYTQESLSHSPGK
c218D9VH2-cIgGBm
[SEQ ID NO: 87]
ELTLQESGPGLVKPSQTLSLTCSFSGFSLSTFGRGVGWIRQRPGRGLEWMGHIWWDDDKYYN
PALKSRISITADTAKNQFSLQLSSMTTEDTAVYYCARIAGGLRRAPYAMDSWGQGTLVTVSS
ASTTAPSVFPLAPSCGSTSGSTVALACLVSGYFPEPVTVSWNSGSLTSGVHTFPSVLQSSGL
YSLSSMVTVPSSRWPSETFTCNVAHPASKTKVDKPVPKRENGRVPRPPDCPKCPAPEMLGGP
SVFIFPPKPKDTLLIARTPEVTCVVVALDPEDPEVQISWFVDGKQMQTAKTQPREEQFAGTY
RVVSVLPIGHQDWLKGKQFTCKVNNKALPSPIERTISKARGQAHQPSVYVLPPSREELSKNT
VSLTCLIKDFFPPDIDVEWQSNGQQEPESKYRTTPPQLDEDGSYFLYSKLSVDKSRWQRGDT
FICAVMHEALHNHYTQESLSHSPGK
c218D9VH3-cIgGBm
[SEQ ID NO: 88]
EVTLQESGPGLVKPSQTLSLTCSFSGFSLSTFGRGVGWIRQRPGRGLEWMGHIWWDDDKYYN
PALKSRLSITKDTAKNQFSLQLSSMTTEDTAVYYCARIAGGLRRAPYAMDSWGQGTLVTVSS
ASTTAPSVFPLAPSCGSTSGSTVALACLVSGYFPEPVTVSWNSGSLTSGVHTFPSVLQSSGL
YSLSSMVTVPSSRWPSETFTCNVAHPASKTKVDKPVPKRENGRVPRPPDCPKCPAPEMLGGP
SVFIFPPKPKDTLLIARTPEVTCVVVALDPEDPEVQISWFVDGKQMQTAKTQPREEQFAGTY
RVVSVLPIGHQDWLKGKQFTCKVNNKALPSPIERTISKARGQAHQPSVYVLPPSREELSKNT
VSLTCLIKDFFPPDIDVEWQSNGQQEPESKYRTTPPQLDEDGSYFLYSKLSVDKSRWQRGDT
FICAVMHEALHNHYTQESLSHSPGK
c218D9VH4-cIgGBm
[SEQ ID NO: 89]
EVTLQESGPGLVKPSQTLSLTCSFSGFSLSTFGRGVGWIRQRPGRGLEWMGHIWWDDDKYYN
PALKSRLSITKDTAKNQVFLQLSSMTTEDTAVYYCARIAGGLRRAPYAMDSWGQGTLVTVSS
ASTTAPSVFPLAPSCGSTSGSTVALACLVSGYFPEPVTVSWNSGSLTSGVHTFPSVLQSSGL
YSLSSMVTVPSSRWPSETFTCNVAHPASKTKVDKPVPKRENGRVPRPPDCPKCPAPEMLGGP
SVFIFPPKPKDTLLIARTPEVTCVVVALDPEDPEVQISWFVDGKQMQTAKTQPREEQFAGTY
RVVSVLPIGHQDWLKGKQFTCKVNNKALPSPIERTISKARGQAHQPSVYVLPPSREELSKNT
VSLTCLIKDFFPPDIDVEWQSNGQQEPESKYRTTPPQLDEDGSYFLYSKLSVDKSRWQRGDT
FICAVMHEALHNHYTQESLSHSPGK
c218D9VL1-cCK
[SEQ ID NO: 90]
EIVMTQSPASLSLSQEEKVTITCRASENIYSSLAWYQQKPGQAPKLLIYAATNLADGVPSRF
SGSGSGTDFSFTISSLEPEDVAVYYCQHFRDTPPTFGQGTKLEIKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c218D9VL2-cCK
[SEQ ID NO: 91]
DIVMTQTPLSLSVSPGETASISCRASENIYSSLAWFRQKPGQSPQLLIYAATNLADGVPDRF
SGSGSGTDFTLRISRVEADDTGVYYCQHFRDTPPTFGQGTKLEIKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c218D9VL3-cCK
[SEQ ID NO: 92]
DIVMTQTPLSLSVSPGETASISCRASENIYSSLAWFRQKPGQSPQLLVYAATNLADGVPDRF
SGSGSGTDYTLRISRVEADDTGVYYCQHFRDTPPTFGQGTKLEIKRNDAQPAVYLFQPSPDQ
LHTGSASVVCLLNSFYPKDINVKWKVDGVIQDTGIQESVTEQDKDSTYSLSSTLTMSSTEYL
SHELYSCEITHKSLPSTLIKSFQRSECQRVD
c218D9VH1
[SEQ ID NO: 93]
ELTLQESGPGLVKPSQTLSLTCVVSGGSVTTFGRGVGWIRQRPGRGLEWMGHIWWDDDKYYN
PALKSRISITADTAKNQFSLQLSSMTTEDTAVYYCARIAGGLRRAPYAMDSWGQGTLVTVSS
c218D9VH2
[SEQ ID NO: 94]
ELTLQESGPGLVKPSQTLSLTCSFSGFSLSTFGRGVGWIRQRPGRGLEWMGHIWWDDDKYYN
PALKSRISITADTAKNQFSLQLSSMTTEDTAVYYCARIAGGLRRAPYAMDSWGQGTLVTVSS
c218D9VH3
[SEQ ID NO: 95]
EVTLQESGPGLVKPSQTLSLTCSFSGFSLSTFGRGVGWIRQRPGRGLEWMGHIWWDDDKYYN
PALKSRLSITKDTAKNQFSLQLSSMTTEDTAVYYCARIAGGLRRAPYAMDSWGQGTLVTVSS
c218D9VH4
[SEQ ID NO: 96]
EVTLQESGPGLVKPSQTLSLTCSFSGFSLSTFGRGVGWIRQRPGRGLEWMGHIWWDDDKYYN
PALKSRLSITKDTAKNQVFLQLSSMTTEDTAVYYCARIAGGLRRAPYAMDSWGQGTLVTVSS
c218D9VL1
[SEQ ID NO: 128]
EIVMTQSPASLSLSQEEKVTITCRASENIYSSLAWYQQKPGQAPKLLIYAATNLADGVPSRF
SGSGSGTDFSFTISSLEPEDVAVYYCQHFRDTPPTFGQGTKLEIK
c218D9VL2
[SEQ ID NO: 129]
DIVMTQTPLSLSVSPGETASISCRASENIYSSLAWFRQKPGQSPQLLIYAATNLADGVPDRF
SGSGSGTDFTLRISRVEADDTGVYYCQHFRDTPPTFGQGTKLEIK
c218D9VL3
[SEQ ID NO: 130]
DIVMTQTPLSLSVSPGETASISCRASENIYSSLAWFRQKPGQSPQLLVYAATNLADGVPDRF
SGSGSGTDYTLRISRVEADDTGVYYCQHFRDTPPTFGQGTKLEIK

### Example 11

### Reactivity of Caninized Antibodies Against Canine IL-31RA

The caninized antibodies were tested for reactivity with canine IL-31RA as follows:

1. Coat 200 ng/well of IL-31RA on an immunoplate and incubate the plate at 4° C. overnight.
2. Wash the plate 3 times with phosphate buffered saline (PBS) containing 0.05% Tween 20 (PBST).
3. Block the plate with 0.5% bovine serum albumin (BSA) in PBS for 45-60 min at room temperature.
4. Wash the plate 3 times with PBST.
5. Three-fold dilute the caninized antibody in each column or row of dilution plate starting at 0.3 μg/mL.
6. Transfer the diluted caninized antibody into each column or row of the immunoplate, and incubate the plate for 45-60 min at room temperature.
7. Wash the plate 3 times with PBST.
8. Add 1:4000 diluted horseradish peroxidase labeled anti-canine IgG Fc into each well of the plate, and then incubate the plate for 45-60 min at room temperature.
9. Wash the plate 3 times with PBST.
10. Add 3,3′,5,5′-tetramethylbenzidine (TMB) Substrate into each well of the plate, and incubate the plate for 10 to 15 min at room temperature to develop the color.
11. Add 100 μL 1.5 M phosphoric acid into each well to stop the reaction. Read plate at 450 nm with 540 nm reference wavelength.

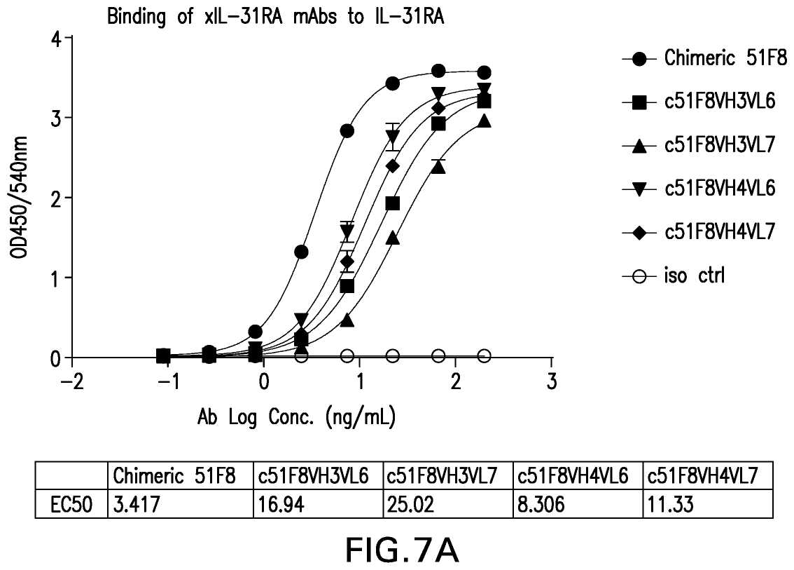

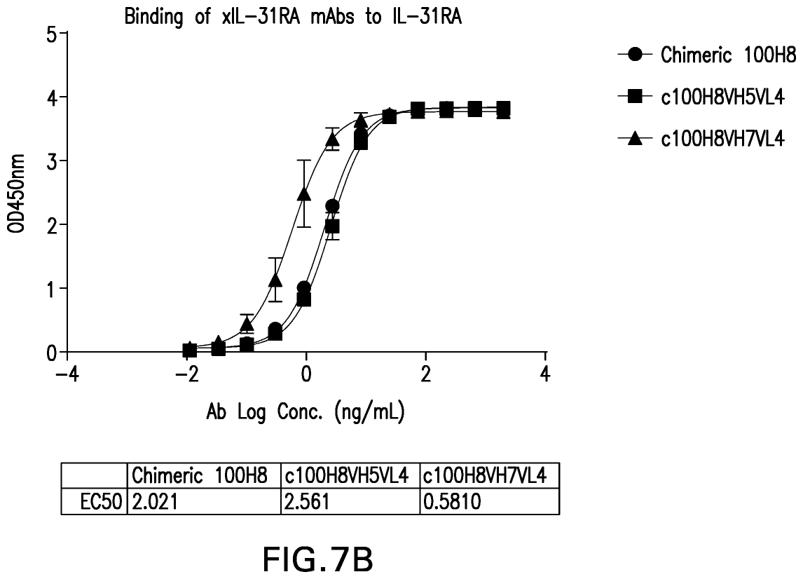

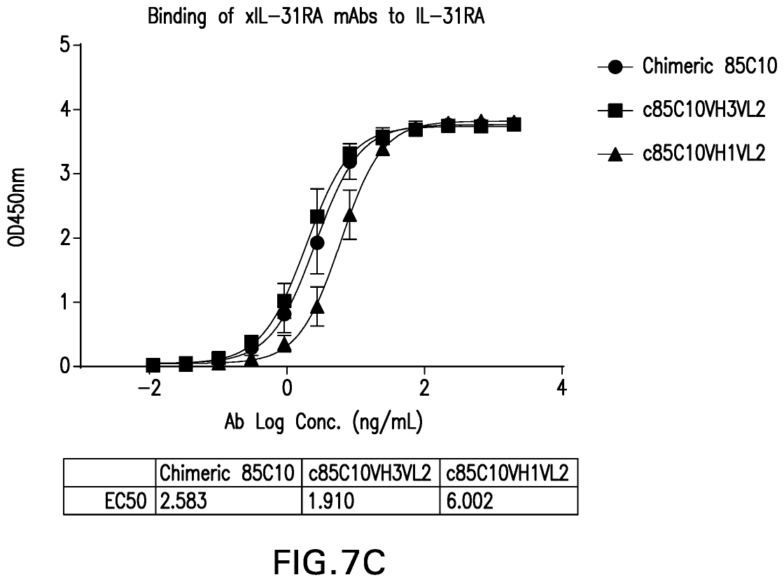

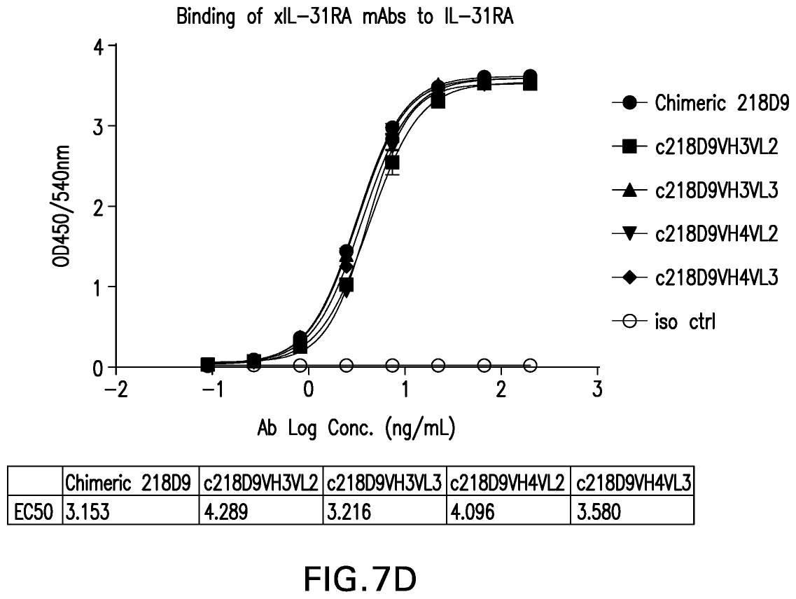

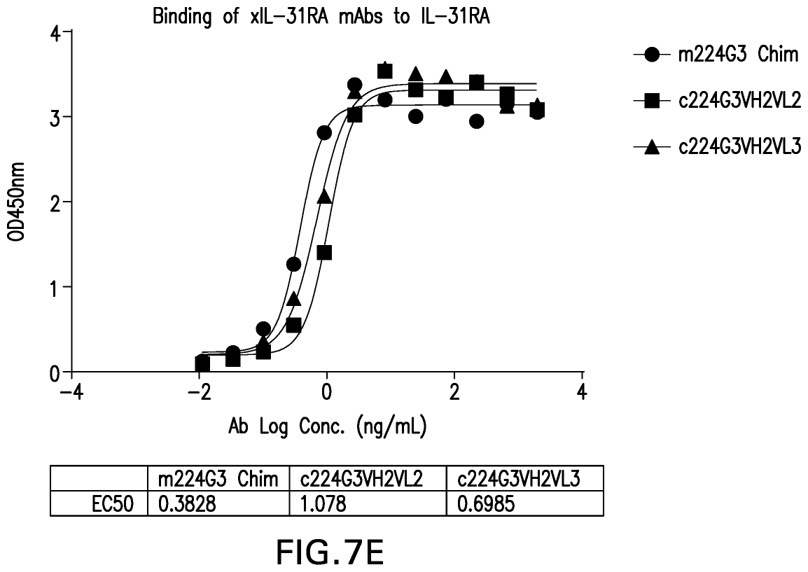

As depicted in FIGS. 7A-7E, the caninized antibodies were tested for their reactivity to canine IL-31RA. The results indicate that the caninized antibodies have similar binding affinity as their corresponding parental antibodies (represented by their chimeric antibodies).

Table 5A below provides the relative binding affinities of the different caninized antibodies (EC50), relative to their corresponding mouse-canine chimera antibody (see, FIGS. 7A-7E). Although these are just relative numbers, caninized antibody 218D9 stands out as an antibody that has essentially the same binding affinity as its parental chimeric murine antibody.

The term “Chimeric” before the antibody number signifies that the antibody is a murine-canine chimeric antibody, e.g., Chimeric 218D9 or Chimeric 51F8. In addition, an “m” before the antibody number followed by a “Chim” signifies that the antibody is a murine-canine chimeric antibody, e.g., m224G3 Chim. The lower case “c” before the antibody number signifies that it is a caninized antibody, e.g., c218D9VH4VL2.]

TABLE 5A
RELATIVE BINDING AFFINITIES OF
THE CANINIZED ANTIBODIES (EC50)

| ANTIBODY | EC50 |
|---|---|
| Chimeric 100H8 | 2.021 |
| c100H8VH5L4 | 2.561 |
| c100H8VH7L4 | 0.581 |
| Chimeric 85C10 | 2.583 |
| c85C10VH3L2 | 1.910 |
| c85C10VH1L2 | 6.002 |
| Chimeric 224G3 | 0.383 |
| c224G3VH2L2 | 1.078 |
| c224G3VH2L3 | 0.699 |
| Chimeric 51F8 | 3.417 |
| c51F8VH3L6 | 16.94 |
| c51F8VH3L7 | 25.02 |
| c51F8VH4L6 | 8.306 |
| c51F8VH4L7 | 11.33 |
| Chimeric 218D9 | 3.153 |
| c218D9VH3L2 | 4.289 |
| c218D9VH3L3 | 3.216 |
| c218D9VH4L2 | 4.096 |

Table 5B below shows the binding constants of the caninized antibodies of 51F8 and 218D9. The results again indicate that caninized 218D9 antibodies have essentially the same binding affinity as their parental chimeric murine antibody, whereas caninized 51F8 antibodies have slightly weaker binding affinity than their parental chimeric murine antibody.

TABLE 5B
BINDING CONSTANTS OF THE CANINIZED
51F8 AND 218D9 ANTIBODIES

| ANTIBODY | KD (M) | ka (1/Ms) | kdis (1/s) |
|---|---|---|---|
| chimeric 51F8 | 2.07E−10 | 6.84E+05 | 1.41E−04 |
| c51F8VH3VL6 | 2.67E−09 | 4.28E+05 | 1.14E−03 |
| c51F8VH3VL7 | 3.99E−09 | 4.01E+05 | 1.60E−03 |
| c51F8VH4VL6 | 1.01E−09 | 4.45E+05 | 4.48E−04 |
| c51F8VH4VL7 | 1.23E−09 | 4.16E+05 | 5.12E−04 |
| chimeric 218D9 | 6.98E−11 | 6.64E+05 | 4.63E−05 |
| c218D9VH3VL2 | 5.28E−10 | 6.41E+05 | 3.38E−04 |
| c218D9VH3VL3 | 4.16E−11 | 6.61E+05 | 2.75E−05 |
| c218D9VH4VL2 | 3.05E−10 | 5.41E+05 | 1.65E−04 |
| c218D9VH4VL3 | 4.47E−11 | 6.01E+05 | 2.69E−05 |

### Example 12

### Mapping of Canine IL-31 Receptor Alpha Epitopes Using Mass Spectroscopy

A method based on chemical crosslinking and mass spectrometry detection was employed to identify epitopes recognized by anti-canine IL-31 receptor alpha mAbs [CovalX Instrument Incorporated, located at 999 Broadway, Suite 305, Saugus, MA 01906-4510]. The application of this technology to epitope mapping of canine IL-31 receptor alpha chain resulted in identification of epitopes recognized by the mAbs listed in Table 6 below. The results from the epitope mapping of canine IL-31 receptor alpha with the antibodies disclosed herein indicate that the mAbs recognize specific peptide epitopes that are present within the extracellular domain of canine IL-31 receptor alpha (see, Table 6 below). The results from the epitope mapping of canine IL-31RA with the eight antibodies included in Table 6, indicate that the mAbs recognize specific peptide epitopes that are present within the extracellular domain of canine IL-31RA. Notably, seven epitopes, which are distributed from N-terminus to C-terminus of IL-3IRA-ECD were identified bound by the eight antibodies. Antibodies 1008, 51F8, 209G5, and 55B3 share epitopes towards the N-terminus, while antibodies 65G9, 85C10 and 224G3 share epitopes towards the C-terminus of the IL-31RA-ECD. Antibody 218D39 has two unique epitopes located at middle portion of IL-31IRA-ECD. As indicated by the functional results, all the eight monoclonal antibodies block IL-31 mediated STAT-3 phosphorylation, meaning that the seven epitopes are important in the interaction of canine IL-3 IRA with its ligand. This exhibits the complexity of the interaction of the canine IL-31 and canine IL-31 receptor complex. Five of the eight monoclonal antibodies tested were caninized, and the positions of their respective epitopes and their identified binding amino acid residues of the IL-3 IRA ECD are both presented in FIGS. 6A-6E and included in Table 6 below [see also, FIGS. 6A-6E which provides the epitopes on canine L-31RA for the antibodies:100H8, 51F8, 218D9, 85C10, and 224G3, respectively, and the position of binding residues of the amino acid sequence of SEQ ID NO: 2 of the respective epitopes on the cIL-31R ECD antigen.]

TABLE 6
IL-31RA EPITOPES RECOGNIZED BY anti-IL-31RA MONOCLONAL ANTIBODIES

| mAb | SEQ ID NO: | EPITOPE SEQUENCE | ¹RESIDUES IDENTIFIED AS BINDING |
|---|---|---|---|
| 100H8 | 97 | YWNLDAIMKIEPPEIFSVK | Y₉₄, K₁₀₂, and K₁₁₂ |
| | 103 | RCAPAESMFWSGWSQEKVGT | R₁₈₃, S₁₉₃, and T₂₀₂ |
| 51F8 | 98 | KIEPPEIFSVKSVLGIKR | K₁₀₂, S₁₁₀, K₁₁₂, K₁₁₈, and R₁₁₉ |
| | 100 | TEYVMTLRCAPAES | T₁₇₆, Y₁₇₈, and S₁₈₉ |
| 209G5 | 98 | KIEPPEIFSVKSVLGIKR | |
| | 101 | YVMTLR | |
| 55B3 | 99 | RPVLAPHSSTLKY | |
| | 102 | RCAPAESM | |
| 218D9 | 104 | RRPVQLMWKKAT | R₂₂₅ and T₂₃₆ |
| | 105 | RIPALNEKT | R₂₉₈, K₃₀₅, and T₃₀₆ |
| 65G9 | 106 | RNGFIKNYTIFY | |
| | 107 | RTSYSLQVMASTNAGGT | |
| 85C10 | 106 | RNGFIKNYTIFY | R₄₃₀, K₄₃₅, T₄₃₈, and Y₄₄₁ |
| | 108 | SLQVMASTNAGGTNGT | S₄₇₂, T₄₇₉, and T₄₈₇ |
| 224G3 | 109 | STNAGGTNGT | S₄₇₈ and T₄₈₇ |

¹Residue position on the cll-31R ECD antigen with the amino acid sequence of SEQ ID
NO: 2.

TABLE 7
SEQUENCES IN FIGS. 6A-6E

| FIG. | SEQ ID NO: | SEQUENCE |
|---|---|---|
| 6A | 119 | SDITYWNLDAIMKIEPPEIFSVKSVLGIKRM |
| | 120 | MTLRCAPAESMFWSGWSQEKVGTTEEEAPYG |
| 6B | 121 | IMKIEPPEIFSVKSVLGIKRM |
| | 122 | TELQAFTEYVMTLRCAPAESM |
| 6C | 123 | AMVDGRRPVQLMWKKATGAPV |
| | 124 | GESPVATLRIPALNEKTFQCI |
| 6D | 125 | RNGFIKNYTIFYQAEDGKEFS |
| | 126^{#} | SYSLQVMASTNAGGTNGTKIN |
| 6E | 126 | SYSLQVMASTNAGGTNGTKIN |

^{#}Also in FIG. 6E.

1. A mammalian antibody or antigen binding fragment thereof that binds canine interleukin-31 receptor alpha (canine IL-31RA), wherein said mammalian antibody or antigen binding fragment thereof comprises a heavy chain comprising three heavy chain complementary determining regions (CDRs), CDR heavy 1 (HCDR1), CDR heavy 2 (HCDR2) and CDR heavy 3 (HCDR3), and wherein said mammalian antibody or antigen binding fragment comprises a light chain comprising three light chain CDRs: CDR light 1 (LCDR1), CDR light 2 (LCDR2), and CDR light 3 (LCDR3); wherein each CDR comprises an amino acid sequence; and
wherein the heavy chain CDRs and the light chain CDRs are selected from the group of sets consisting of (i), (ii), (iii), (iv), (v), and (vi);
wherein for set (i):
HCDR1 comprises the amino acid sequence of SEQ ID NO:4;
HCDR2 comprises the amino acid sequence of SEQ ID NO:14;
HCDR3 comprises the amino acid sequence of SEQ ID NO:24;
LCDR1 comprises the amino acid sequence of SEQ ID NO:33;
LCDR2 comprises the amino acid sequence of SEQ ID NO:37; and
LCDR3 comprises the amino acid sequence of SEQ ID NO:46;
wherein for set (ii):
HCDR1 comprises the amino acid sequence of SEQ ID NO:5;
HCDR2 comprises the amino acid sequence of SEQ ID NO:15;
HCDR3 comprises the amino acid sequence of SEQ ID NO:25;
LCDR1 comprises the amino acid sequence of SEQ ID NO:32;
LCDR2 comprises the amino acid sequence of SEQ ID NO:41; and
LCDR3 comprises the amino acid sequence of SEQ ID NO:47;
wherein for set (iii)
HCDR1 comprises the amino acid sequence of SEQ ID NO:6;
HCDR2 comprises the amino acid sequence of SEQ ID NO:16;
HCDR3 comprises the amino acid sequence of SEQ ID NO:26;
LCDR1 comprises the amino acid sequence of SEQ ID NO:32;
LCDR2 comprises the amino acid sequence of SEQ ID NO:42; and
LCDR3 comprises the amino acid sequence of SEQ ID NO:48;
wherein for set (iv):
HCDR1 comprises the amino acid sequence of SEQ ID NO:7;
HCDR2 comprises the amino acid sequence of SEQ ID NO:17;
HCDR3 comprises the amino acid sequence of SEQ ID NO:27;
LCDR1 comprises the amino acid sequence of SEQ ID NO:34;
LCDR2 comprises the amino acid sequence of SEQ ID NO:37; and
LCDR3 comprises the amino acid sequence of SEQ ID NO:50;
wherein for set (v):
HCDR1 comprises the amino acid sequence of SEQ ID NO:8;
HCDR2 comprises the amino acid sequence of SEQ ID NO:18;
HCDR3 comprises the amino acid sequence of SEQ ID NO:28;
LCDR1 comprises the amino acid sequence of SEQ ID NO:35;
LCDR2 comprises the amino acid sequence of SEQ ID NO:43; and
LCDR3 comprises the amino acid sequence of SEQ ID NO:51; and
wherein for set (vi)
HCDR1 comprises the amino acid sequence of SEQ ID NO:9;
HCDR2 comprises the amino acid sequence of SEQ ID NO:19;
HCDR3 comprises the amino acid sequence of SEQ ID NO:29;
LCDR1 comprises the amino acid sequence of SEQ ID NO:36;
LCDR2 comprises the amino acid sequence of SEQ ID NO:44; and
LCDR3 comprises the amino acid sequence of SEQ ID NO:49.

2. The mammalian antibody or antigen binding fragment thereof of claim 1, wherein the mammalian antibody or antigen binding fragment thereof binds canine IL-31RA and blocks the binding of canine IL-31RA to canine interleukin-31.

3.-10. (canceled)

11. The mammalian antibody or antigen binding fragment thereof of claim 1, wherein the mammalian antibody or antigen binding fragment thereof is a caninized IL-31RA antibody or a caninized antigen binding fragment thereof.

12. (canceled)

13. The mammalian antibody or antigen binding fragment thereof of claim 11, comprising a canine fragment crystallizable region (cFc); wherein the cFc comprises an amino acid sequence having at least 90%, 95%, 98%, or 99% identity, or having 100% identity, with an amino acid sequence selected from the group consisting of SEQ ID NO:110, SEQ ID NO:116, SEQ ID NO:117, SEQ ID NO:118, and SEQ ID NO:111.

14. (canceled)

15. The mammalian antibody or antigen binding fragment thereof of claim 1, wherein the mammalian antibody or antigen binding fragment thereof is a caninized IL-31RA antibody or a caninized antigen binding fragment thereof and wherein the caninized antibody comprises (a)_a heavy chain variable region comprising an amino acid sequence selected from the group consisting of SEQ ID NO:93, SEQ ID NO:94, SEQ ID NO:95, and SEQ ID NO:96; (b) a light chain variable region comprising an amino acid sequence selected from the group consisting of SEQ ID NO:128, SEQ ID NO:129, and SEQ ID NO:130; or (c) both the heavy chain variable region comprising the amino acid sequence selected from the group consisting of SEQ ID NO:93, SEQ ID NO:94, SEQ ID NO:95, and SEQ ID NO:96 and the light chain variable region comprising the amino acid sequence selected from the group consisting of SEQ ID NO:128, SEQ ID NO:129, and SEQ ID NO:130.

16. (canceled)

17. The mammalian antibody or antigen binding fragment thereof of claim 15, wherein the light chain comprises an amino acid sequence selected from the group consisting of SEQ ID NO:90, SEQ ID NO:91, and SEQ ID NO:92.

18. The mammalian antibody or antigen binding fragment thereof of claim 15, wherein
the heavy chain variable region comprises the amino acid sequence of SEQ ID NO:95 and the light chain variable region comprises the amino acid sequence of SEQ ID NO:129, or
the heavy chain variable region comprises the amino acid sequence of SEQ ID NO:95 and the light chain variable region comprises the amino acid sequence of SEQ ID NO:130, or
the heavy chain variable region comprises the amino acid sequence of SEQ ID NO:96 and the light chain variable region comprises the amino acid sequence of SEQ ID NO:129, or
the heavy chain variable region comprises the amino acid sequence of SEQ ID NO:96 and the light chain variable region comprises the amino acid sequence of SEQ ID NO:130.
the heavy chain variable region comprises the amino acid sequence of SEQ ID NO:95 and the light chain comprises the amino acid sequence of SEQ ID NO:91, or
the heavy chain variable region comprises the amino acid sequence of SEQ ID NO:95 and the light chain comprises the amino acid sequence of SEQ ID NO:92, or
the heavy chain variable region comprises the amino acid sequence of SEQ ID NO:96 and the light chain comprises the amino acid sequence of SEQ ID NO:91, or
the heavy chain variable region comprises the amino acid sequence of SEQ ID NO:96 and the light chain comprises the amino acid sequence of SEQ ID NO:92.

19. The mammalian antibody or antigen binding fragment thereof of claim 15 comprising a hinge region that comprises an amino acid sequence having at least 90% or 95% identity, or having 100% identity, with an amino acid selected from the group consisting of SEQ ID NO:112, SEQ ID NO:113, SEQ ID NO:114, and SEQ ID NO:115.

20. The mammalian antibody or antigen binding fragment thereof of claim 15, comprising a canine fragment crystallizable region (cFc); wherein the cFc comprises an amino acid sequence having at least 90%, 95%, 98%, or 99% identity, or having 100% identity, with an amino acid sequence selected from the group consisting of SEQ ID NO:110, SEQ ID NO:116, SEQ ID NO:117, SEQ ID NO:118, and SEQ ID NO:111.

21. (canceled)

22. The mammalian antibody or antigen binding fragment thereof of claim 15, wherein the light chain comprises an amino acid sequence selected from the group consisting of SEQ ID NO:90, SEQ ID NO:91, and SEQ ID NO:92; and wherein the heavy chain comprises an amino acid sequence selected from the group consisting of SEQ ID NO:86, SEQ ID NO:87, SEQ ID NO:88, and SEQ ID NO:89.

23. The mammalian antibody or antigen binding fragment thereof of claim 22, wherein
the heavy chain comprises the amino acid sequence of SEQ ID NO:88 and the light chain comprises the amino acid sequence of SEQ ID NO:91, or
the heavy chain comprises the amino acid sequence of SEQ ID NO:88 and the light chain comprises the amino acid sequence of SEQ ID NO:92, or
the heavy chain comprises the amino acid sequence of SEQ ID NO:89 and the light chain comprises the amino acid sequence of SEQ ID NO:91, or
the heavy chain comprises the amino acid sequence of SEQ ID NO:89 and the light chain comprises the amino acid sequence of SEQ ID NO:92.

24. The mammalian antibody or antigen binding fragment thereof of claim 15, wherein the mammalian antibody or antigen binding fragment thereof is a caninized IL-31RA antibody or a caninized antigen binding fragment thereof, and wherein the heavy chain comprise an amino acid sequence selected from the group consisting of SEQ ID NO:72, SEQ ID NO:73, SEQ ID NO:74, and SEQ ID NO:75.

25. The mammalian antibody or antigen binding fragment thereof of claim 24, wherein the light chain comprises an amino acid sequence selected from the group consisting of SEQ ID NO:76, SEQ ID NO:77, and SEQ ID NO:78.

26. The mammalian antibody or antigen binding fragment thereof of claim 25, wherein
the heavy chain comprises the amino acid sequence of SEQ ID NO:74 and the light chain comprises the amino acid sequence of SEQ ID NO:77, or
the heavy chain comprises the amino acid sequence of SEQ ID NO:74 and the light chain comprises the amino acid sequence of SEQ ID NO:78, or
the heavy chain comprises the amino acid sequence of SEQ ID NO:75 and the light chain comprises the amino acid sequence of SEQ ID NO:77, or
the heavy chain comprises the amino acid sequence of SEQ ID NO:75 and the light chain comprises the amino acid sequence of SEQ ID NO:78.

27. A canine or caninized antibody or antigen binding fragment thereof that binds to canine interleukin-31 receptor alpha (canine IL-31RA), wherein when bound to canine IL-31RA, the canine or caninized antibody or antigen binding fragment thereof binds to an epitope comprising an amino acid sequence selected from the group consisting of SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109 or any combination thereof; and wherein the canine or caninized antibody or antigen binding fragment thereof binds to canine IL-31RA and blocks the binding of canine IL-31RA to canine IL-31.

28. (canceled)

29. A nucleic acid that encodes at least one selected from the group consisting of:
(a) the heavy chain,
(b) a heavy chain variable region,
(c) the light chain,
(d) a light chain variable region,
(e) both the heavy chain and the light chain, and
(f) both a heavy chain variable region and a light chain variable region, of the mammalian antibody or antigen binding fragment thereof of claim 11.

30. (canceled)

31. (canceled)

32. An expression vector comprising one or more of the nucleic acids of claim 29.

33. A vector system comprising a pair of the expression vectors of claim 32, wherein
(a) one of the pair of expression vectors encodes the light chain of the caninized antibody and the other of the pair of expression vectors encodes the heavy chain of said caninized antibody; or
(b) one of the pair of expression vectors encodes the light chain variable region of the caninized antibody and the other of the pair of expression vectors encodes the heavy chain variable region of the caninized antibody.

34. A host cell comprising the one or more of the expression vectors of claim 32.

35. A pharmaceutical composition comprising the mammalian antibody or antigen binding fragment thereof of claim 11, and a pharmaceutically acceptable carrier or diluent.

36. A method of aiding in blocking pruritus associated with atopic dermatitis in a canine, comprising administering to the canine in need thereof a therapeutically effective amount of the pharmaceutical composition of claim 35.

37. (canceled)

38. The mammalian antibody or antigen binding fragment thereof of claim 15, wherein the heavy chain comprises an amino acid sequence selected from the group consisting of SEQ ID NO:86, SEQ ID NO:87, SEQ ID NO:88, and SEQ ID NO:89.
